SEQUENCE LISTING (1 sequence)

SEQ ID NO: 1 - DNA, 21 bp, Artificial Sequence
  Synthetic
aagacccgcgccgaggtgaag

(19) United States
(12) Patent Application Publication
(10) Pub. No.: US 2012/0302629 A1
(43) Pub. Date: Nov. 29, 2012

(54) BIOMARKERS FOR GOSSYPOL CHEMOTHERAPY AND METHODS OF TREATING DISEASE

(75) Inventors: Shaomeng Wang, Saline, MI (US); Feng Jiang, Ann Arbor, MI (US); Jianting Long, Guangzhou (CN); Longchuan Bai, Ann Arbor, MI (US)

(73) Assignee: THE REGENTS OF THE UNIVERSITY OF MICHIGAN, Ann Arbor, MI (US)

(21) Appl. No.: 13/388,610

(22) PCT Filed: Jul. 28, 2010
(86) PCT No.: PCT/US2010/043477

§ 371 Date: Apr. 17, 2012

Related U.S. Application Data
(60) Provisional application No. 61/232,205, filed on Aug. 7, 2009.

(51) Int. Cl. A61K 31/11; C12Q 1/68; A61P 35/00; A61K 31/337; A61P 29/00; A61P 31/00; A61P 7/00; A61P 35/02; G01N 33/574; A61P 37/00

(52) U.S. Cl. 514/449; 435/7.9; 435/6.11; 514/700

(57) ABSTRACT

The present invention provides a biomarker for selecting a patient for treatment with gossypol, wherein the biomarker comprises an elevated expression level of c-Myc, Mcl-1, or combination thereof, relative to the normal expression level of c-Myc, Mcl-1, or combination thereof. The present invention also provides methods for targeting patients for treatment with gossypol, wherein the patient has a disease, condition, or disorder that overexpresses c-Myc, Mcl-1, or combination thereof. The present invention also provides methods for treating or ameliorating a disease, condition, or disorder in a patient comprising determining the expression level of c-Myc, Mcl-1, or combination thereof in the patient and administering gossypol to the patient. In certain embodiments of the invention, the disease is cancer, and the cancer cells show elevated expression levels of c-Myc compared to non-cancerous cells. The invention also provides methods for overcoming Mcl-1-mediated chemoresistance comprising administering gossypol to a patient in need thereof.

### CROSS REFERENCE TO RELATED APPLICATION

The present invention claims priority to pending U.S. Provisional Patent Application No. 61/232,205, filed Aug. 7, 2009, the contents of which are hereby incorporated by reference in its entirety.

### BACKGROUND OF THE INVENTION

1. Field of the Invention

The present invention relates generally to the fields of biomarkers, treatment of disease, and chemoresistance. Specifically, the invention provides a biomarker for selecting a patient for treatment with gossypol, or for treatment with gossypol and one or more additional therapeutic agents, wherein the biomarker comprises an elevated expression level of c-Myc, Mcl-1, or combination thereof. The invention also provides methods for targeting patients for treatment with gossypol, or for treatment with gossypol and one or more additional therapeutic agents, wherein the patient has a disease, condition, or disorder that overexpresses c-Myc, Mcl-1, or combination thereof. The invention also provides methods for treating or ameliorating a disease, condition, or disorder in a patient comprising determining the expression level of c-Myc, Mcl-1, or combination thereof in the patient and administering gossypol, or gossypol and one or more additional therapeutic agents, to the patient if the patient has an elevated expression level of c-Myc, Mcl-1, or combination thereof. In certain embodiments of the invention, the disease is a hyperproliferative disease such as cancer, and the cancer cells show elevated expression levels of c-Myc compared to non-cancerous cells. The invention also provides methods for overcoming Mcl-1-mediated chemoresistance comprising administering gossypol to a patient in need thereof.

2. Related Art

The aggressive cancer cell phenotype is the result of a variety of genetic and epigenetic alterations leading to deregulation of intracellular signaling pathways (Ponder, Nature 411:336 (2001)). The commonality for all cancer cells, however, is their failure to execute an apoptotic program, and lack of appropriate apoptosis due to defects in the normal apoptosis machinery is a hallmark of cancer (Lowe et al., Carcinogenesis 21:485 (2000)). Most of the current cancer therapies, including chemotherapy, radiation (radiotherapy), and immunotherapy, work by indirectly inducing apoptosis in cancer cells. The inability of cancer cells to execute an apoptotic program due to defects in the normal apoptotic machinery is thus often associated with an increase in resistance to chemotherapy, radiation, or immunotherapy-induced apoptosis. Primary or acquired resistance of human cancer of different origins to current treatment protocols due to apoptosis defects is a major problem in current cancer therapy (Lowe et al., Carcinogenesis 21:485 (2000); Nicholson, Nature 407:810 (2000)). Accordingly, current and future efforts towards designing and developing new molecular target-specific anticancer therapies to improve survival and quality of life of cancer patients must include strategies that specifically target cancer cell resistance to apoptosis. In this regard, targeting crucial negative regulators that play a central role in directly inhibiting apoptosis in cancer cells represents a highly promising therapeutic strategy for new anticancer drug design.

Two classes of central negative regulators of apoptosis have been identified. The first class of regulators is the inhibitor of apoptosis proteins (IAPB) (Deveraux et al., Genes Dev. 13:239 (1999); Salvesen et al., Nat. Rev. Mol. Cell. Biol. 3:401 (2002)). IAP proteins potently suppress apoptosis induced by a large variety of apoptotic stimuli, including chemotherapeutic agents, radiation, and immunotherapy in cancer cells.

The second class of central negative regulators of apoptosis is the Bcl-2 family of proteins (Adams et al., Science 281:1322 (1998); Reed, Adv. Pharmacol. 41:501 (1997); Reed et al., J. Cell. Biochem. 60:23 (1996)). Bcl-2 is the founding member of the family and was first isolated as the product of an oncogene. The Bcl-2 family now includes both anti-apoptotic molecules such as Bcl-2 and Bcl-X_{L} and pro-apoptotic molecules such as Bax, Bak, Bid, and Bad. Bcl-2 and Bcl-X_{L} are overexpressed in many types of human cancer (e.g., breast, prostate, colorectal, lung, etc.), including Non-Hodgkin's lymphoma, which is caused by a chromosomal translocation (t14, 18) that leads to overexpression of Bcl-2. This suggests that many cancer cell types depend on the elevated levels of Bcl-2 and/or Bcl-X_{L} to survive the other cellular derangements that simultaneously both define them as cancerous or pre-cancerous cells and cause them to attempt to execute the apoptosis pathway. Also, increased expression of Bcl-2 family proteins has been recognized as a basis for the development of resistance to cancer therapeutic drugs and radiation that act in various ways to induce cell death in tumor cells.

Bcl-2 and Bcl-X_{L} are thought to play a role in tumor cell migration and invasion, and therefore, metastasis (Amberger et al., Cancer Res. 58:149 (1998); Wick et al., FEBS Lett. 440:419 (1998); Mohanam et al., Cancer Res. 53:4143 (1993); Pedersen et al., Cancer Res. 53:5158 (1993)). Bcl-2 family proteins appear to provide tumor cells with a mechanism for surviving in new and non-permissive environments (e.g., metastatic sites), and contribute to the organospecific pattern of clinical metastatic cancer spread (Rubio, Lab Invest. 81:725 (2001); Fernandez et al., Cell Death Differ. 7:350 (2000)). Anti-apoptotic proteins such as Bcl-2 and/or Bcl-X_{L} are also thought to regulate cell-cell interactions, for example through regulation of cell surface integrins (Reed, Nature 387:773 (1997); Frisch et al., Curr. Opin. Cell Biol. 9:701 (1997); Del Bufalo et al., FASEB J. 11:947 (1997)).

Therapeutic strategies for targeting Bcl-2 and Bcl-X_{L} in cancer to restore cancer cell sensitivity and overcome resistance of cancer cells to apoptosis have been extensively reviewed (Adams et al., Science 281:1322 (1998); Reed, Adv. Pharmacol. 41:501 (1997); Reed et al., J. Cell. Biochem. 60:23 (1996)).

The c-myc proto-oncogene encodes a transcription factor, c-Myc, that regulates a variety of important cellular processes such as apoptosis, cell growth and proliferation, cell-cycle progression, transcription, differentiation, cell metabolism, angiogenesis, and cell motility (Vita et al., Seminars in Cancer Biology 16:318 (2006); Robson et al., Recent Patents on Anti-Cancer Drug Discovery 1:305 (2006); Amati et al., Frontiers in Bioscience 3:250 (1998)). This makes c-Myc an attractive therapeutic target for anticancer therapies (Hermeking, Current Cancer Drug Targets 3:163 (2003); Pelengaris et al., Expert Opin. Ther. Targets 7:623 (2003); Prochownik, Expert Rev. Anticancer Ther. 4:289 (2004)). Other members of the myc family that show oncogenic activity include MYCN and MYCL1 (Ponzielli et al., European Journal of Cancer 41:2485 (2005)).

c-Myc expression is tightly controlled in normal cells. But c-Myc is frequently deregulated in cancerous cells (e.g., tumor-derived cells) through a variety of mechanisms (Pendergast, Oncogene 18:2967 (1999)). c-Myc deregulation (e.g., rearrangement, amplification, overexpression and/or translocation) has been reported in hematological malignancies such as B-acute lymphocytic leukemia, Burkitt's lymphoma, diffuse large cell lymphoma, multiple myeloma, primary plasma leukemia, and solid tumors such as atypical carcinoid lung cancer, bladder cancer, breast cancer, cervix cancer, colon cancer, gastric cancer, glioblastoma, heptocellular carcinoma, large cell neuroendocrine carcinoma, medulloblastoma, melanoma (nodular), melanoma (superficial spreading) neuroblastoma, oesophageal squamous cell carcinoma, osteosarcoma, ovarian cancer, prostate cancer, renal clear cell carcinoma, and small cell lung carcinoma (Vita et al., Seminars in Cancer Biology 16:318 (2006) and references cited therein). Elevated expression levels (i.e., overexpression) of c-Myc are found in Burkitt's lymphoma, breast cancer, colon cancer, gastric cancer, medulloblastoma, ovarian cancer, and prostate cancer with frequencies of 91%, 45%, 67%, 47%, 31%, 44%, and 70%, respectively (Vita et al., Seminars in Cancer Biology 16:318 (2006) and references cited therein).

The precise roles that c-Myc provides to provoke tumorigenesis are not fully resolved but do include the regulation of target genes that control cell division, differentiation, cell size, and angiogenesis (Maclean et al., Molecular and Cellular Biology 7256:23 (2003); Evan et al., Science 281:1317 (1998)).

Gossypol is a natural polyphenolic compound from cotton seeds and roots. Gossypol was initially developed as an oral contraceptive drug in men in the 1970s in China and later in Brazil (Wu, Drugs 38:333 (1989)). Subsequently, gossypol was shown to have broad anti-cancer activity (Dodou et al., Expert Opinion on Investigational Drugs 14:1419 (2005)). Gossypol is a racemic compound existing as two enantiomers, namely (+)-gossypol and (−)-gossypol. (−)-Gossypol is the more potent enantiomer for anticancer activity (Wang et al., Semin. Oncol. 30(5 Suppl 16):133 (2003); Liu et al., Anticancer Res. 22(1A):33 (2002)). In preclinical studies, (−)-gossypol was shown to effectively inhibit cancer cell growth and induce cell death/apoptosis in a variety of cancer cell lines and tumor types (Dodou et al., Expert Opinion on Investigational Drugs 14:1419 (2005)). (−)-Gossypol also demonstrated single agent activity in xenograft models of human cancer and significantly enhanced the antitumor activity of other therapeutic agents, including radio- and chemotherapies (Xu et al., Molecular Cancer Therapeutics 4:197 (2005); Wolter et al., Neoplasia 8:163 (2006); Oliver et al., Clin. Cancer Res. 10:7757 (2004); Bauer J A, Trask D K, Kumar B, et al., Molecular Cancer Therapeutics 4:1096 (2005); Mohammad et al., Mol. Cancer. Ther. 4:13 (2005); Meng et al., Leuk. Lymphoma 48:2204 (2007)). (−)-Gossypol also delays the onset of androgen-independent prostate cancer upon castration in mice model (Loberg et al., Neoplasia 9:1030 (2007)).

Clinical trials have been conducted to evaluate racemic gossypol in several cancer types (Flack et al., The Journal of Clinical Endocrinology and Metabolism 76:1019 (1993); Van Poznak et al., Breast Cancer Research and Treatment 66:239 (2001); Manion et al., Curr. Opin. Investig. Drugs 7:1077 (2006)). A low but measurable response was observed for malignant gliomas (Brandes et al., Anticancer Research 20:1913 (2000); Bushunow et al., Journal of Neuro-Oncology 43:79 (1999)). Response to racemic gossypol was also observed in metastatic adrenal cancer patients who had failed other chemotherapeutic agents (Flack et al., The Journal of Clinical Endocrinology and Metabolism 76:1019 (1993)). The clinical activity of racemic gossypol in patients with refractory metastatic breast cancer was limited (Van Poznak et al., Breast Cancer Research and Treatment 66:239 (2001)). Since (−)-gossypol is the active enantiomer, it was recently evaluated as a new anticancer agent and has demonstrated antitumor activity and manageable toxicity as a single agent in clinical trials. (−)-Gossypol is undergoing further clinical evaluation either alone or in combination with chemotherapeutic agents for the treatment of androgen-independent prostate cancer and other forms of cancer.

Numerous studies have been performed to investigate the cellular mechanism of action of gossypol and its enantiomers for their antitumor activity and their possible molecular targets (Dodou et al., Expert Opinion on Investigational Drugs 14:1419 (2005)). Several enzymes, including hexokinase and lactate dehydrogenase, GST and lipooxygenase, protein kinase C, and nuclear enzymes such as DNA polymerase topoisomerase II have been shown to be inhibited by gossypol and its enantiomers (Dodou et al., Expert Opinion on Investigational Drugs 14:1419 (2005)). Whether or not inhibition of these enzymes by gossypol contributes to its antitumor activity, however, has not been established. One study has shown that mitochondria targeting by (−)-gossypol may play a role for its antitumor activity (Benz et al., Mol. Pharmacol. 37:840 (1990)). Using electron microscopy, it was demonstrated that the earliest ultrastructural change in tumor cells exposed to (−)-gossypol is the selective destruction of their mitochondria but not other organelle (Benz et al., Mol. Pharmacol. 37:840 (1990)). The magnitude of these antimitochondrial effects correlates with the antiproliferative activity of (−)-gossypol. This study provided strong evidence that the mitochondria damage may play a key role for the antitumor activity of (−)-gossypol, although the molecular targets were not determined.

Research in the last decade has established that mitochondria play a key role in apoptosis and that Bcl-2 family proteins are central mediators of apoptosis by regulation of mitochondria integrity (Danial et al., Cell 116:205 (2004); Adams et al., Oncogene 26:1324 (2007); Letai, Trends Mol. Med. 11:442 (2005)). (−)-Gossypol binds to several anti-apoptotic Bcl-2 family proteins, including Bcl-2, Bcl-xL and Mcl-1 in their Bcl-2 homology 3 (BH3) groove where pro-apoptotic Bcl-2 proteins bind, functioning as a BH3 mimetic (Wang et al., Semin. Oncol. 30(5 Suppl 16):133 (2003); Wang et al., Journal of Medicinal Chemistry 49:6139 (2006)). Consistent with its targeting of these anti-apoptotic Bcl-2 proteins, (−)-gossypol induces apoptosis in a variety of cancer cell lines (Xu et al. Molecular Cancer Therapeutics 4:197 (2005); Oliver et al., Clin Cancer Res 10:7757 (2004); Mohammad et al., Mol Cancer Ther 4:13 (2005); Huang et al., Anticancer Res 26:1925 (2006)); Meng et al., Leuk. Lymphoma 48:2204 (2007)). (−)-Gossypol also directly induces release of cytochrome c from isolated mitochondria (Oliver et al., Molecular Cancer Therapeutics 4:23 (2005)). (−)-Gossypol antagonizes Bcl-2 and Bcl-xL in Jurkat T-leukemia cells and overcomes Mcl-1-mediated resistance to bortezomib in melanoma cells (Oliver et al., Molecular Cancer Therapeutics 4:23 (2005); Wolter et al., Cell Death and Differentiation 14:1605 (2007); Gomez-Bougie at al., Cancer Res. 67:5418 (2007)). Treatment of cancer cells by (−)-gossypol was shown to disrupt the heterodimerization of Bcl-xL with Bim (Mohammad et al., Mol. Cancer. Ther. 4:13 (2005)). Collectively, these studies have provided evidence that (−)-gossypol induces apoptosis in tumor cells, at least in part, by targeting Bcl-2, Bcl-xL and Mcl-1 to disrupt mitochondrial integrity.

Most approaches for the treatment of cancer involve non-specific chemotherapeutic agents, radiotherapeutic agents, and/or surgical techniques. These treatment options often result in unwanted side effects and are not always effective in treating or ameliorating the cancer. It would be desirable to be able to select patients who are particularly likely to respond favorably to treatment with an anticancer agent, such as gossypol. Thus, there exists a need for at least one biomarker that indicates whether or not administration of gossypol to a patient, either alone or in combination with one or more additional therapeutic agents, would effectively treat or ameliorate the cancer.

Likewise, it would be desirable to be able to select patients having diseases other than cancer (e.g., other hyperproliferative diseases, autoimmune diseases, inflammatory diseases, infectious diseases, degenerative diseases, vascular diseases, etc.) for treatment with gossypol, wherein the administration of gossypol would have high probability of successfully treating the disease.

As mentioned above, an obstacle in chemotherapy can be the development of chemoresistance of certain cancer cells to certain chemotherapeutic agents, which reduces or negates the effectiveness of many chemotherapeutic agents. Such resistance is often linked to the inability of the chemotherapeutic agents to induce apoptosis in particular cancer cells. Counteracting chemoresistance can restore efficacy of many chemotherapeutic agents, and can help lower the dosage of these agents, thereby alleviating or avoiding unwanted side effects of these agents. Chemoresistance has, in several instances, been linked to dysregulated expression, or alterations of anti-apoptotic Bcl-2 proteins, such as Mcl-1, and subsequent alterations to various signal transducing pathways. Thus, there exists a need to counteract chemoresistance of tumor cells to chemotherapeutic agents, such as Mcl-1-mediated chemoresistance, and improve the efficacy of chemotherapeutic agents.

### SUMMARY OF THE INVENTION

The present invention is based in part on the discovery that c-Myc plays a pivotal role in mediating apoptosis induction by (−)-gossypol. Thus, the present invention provides a biomarker for selecting a patient for treatment with gossypol, or for treatment with gossypol with one or more additional therapeutic agents, wherein said biomarker comprises an elevated expression level of c-Myc.

Since c-Myc expression plays a pivotal role in mediating apoptosis induction by (−)-gossypol, the present invention also provides methods for selecting or assessing the suitability of a patient with a disease, condition, or disorder responsive to the treatment with gossypol comprising determining the expression level of c-Myc in said patient.

The present invention is also based in part on the discovery that expression of c-Myc facilitates gossypol-induced apoptosis. Thus, the present invention provides methods for treating or ameliorating a disease, condition, or disorder in a patient comprising: (a) determining the expression level of c-Myc in said patient; and (b) administering gossypol to said patient if said patient has an elevated expression level of c-Myc relative to the normal expression level of c-Myc.

The present invention is also based in part on the discovery that (−)-gossypol induces upregulation of pro-apoptotic Noxa and Puma proteins in cancer cell lines. Noxa is upregulated by (−)-gossypol in tumor cells but not in normal cells. It was shown that (−)-gossypol overcomes resistance of certain Mcl-1-expressing cancer cells to ABT-737, at least in part through upregulation of Noxa. Thus, the present invention provides methods for overcoming Mcl-1-mediated resistance to certain chemotherapeutic agents in a patient comprising: (a) determining if said patient develops chemoresistance or is chemoresistant to said one or more chemotherapeutic agents, wherein said one or more chemotherapeutic agents are not gossypol; and (b) co-administering gossypol to said patient if said patient develops said chemoresistance or is chemoresistant to said one or more chemotherapeutic agents.

Other features and advantages of the invention will be apparent from the following detailed description, and from the claims. The disclosed materials, methods, and examples are for illustrative purposes only and are not intended to be limiting. Skilled artisans will appreciate that methods and materials similar or equivalent to those described herein can be used to practice the invention.

Unless otherwise defined, all technical and scientific terms used herein have the meaning commonly understood by one skilled in the art to which this invention belongs. All publications, patent applications, patents, and other references mentioned herein are incorporated by reference in their entirety. In case of conflict, the present specification, including definitions, will control.

### BRIEF DESCRIPTION OF FIGURES

FIG. 1 is a bar graph and a series of illustrations that show cell death induction by (−)-gossypol in 2LMP cells. 2LMP cells were treated with (−)-gossypol at the indicated dose for 24 h. Cell death was analyzed by trypan blue dye exclusion and whole cell lysate were analyzed by western blot.

FIG. 2 is two bar graphs and a series of illustrations that show apoptosis induction by (−)-gossypol in 2LMP (A) and PC-3 cells (B). Cells were treated at 10 μM of (−)-gossypol at the indicated time points. Cell death was analyzed by trypan blue dye exclusion and whole cell lysate were analyzed by western blot.

FIG. 3 is two bar graphs that show cell death induction by (−)-gossypol in 2LMP cells. 2LMP cells were treated with (−)-gossypol or Trail at the indicated time and dose. Caspase-9 and -3 activities were analyzed as described in Example 1.

FIG. 4 is a line graph and series of illustrations that show apoptosis induction by (−)-gossypol in both Bax/Bak dependent and independent manners. 2LMP (C) cells were transfected with SiRNAs for negative control (SiGFP) or Bax and Bak. 24 h later, cells were treated with or without (−)-gossypol for 18 h, followed by flow cytometric analysis of Annexin V/PI staining. Three independent experiments were performed and a representative apoptotic index was plotted. Whole cell lysates were analyzed by western blot.

FIG. 5 is a line graph and a series of illustrations that show apoptosis induction by (−)-gossypol in both Bax/Bak dependent and independent manners. PC-3 cells were transfected with SiRNAs for negative control (Si-GFP) or Bax and Bak. 24 h later, cells were treated with or without (−)-gossypol for 18 h, followed by flow cytometric analysis of Annexin V/PI staining. Three independent experiments were performed and a representative apoptotic index was plotted. Whole cell lysates were analyzed by western blot.

FIG. 6 is a line graph and a series of illustrations that show cell death induction by (−)-gossypol in DU145 cells. DU145 cells were transfected with SiRNAs for Si-GFP or Bax and Bak. After 24 h, cells were treated with or without (−)-gossypol for 18 h. Cell death was analyzed by trypan blue dye exclusion. Whole cell lysate were analyzed by western blot.

FIG. 7 is two line graphs and a series of panels that show cell death induction by (−)-gossypol in MEF cells. Wild type MEF (WT) and Bax/Bak double knock-out MEF (Bax/Bak-KO) were treated with (−)-gossypol for 24 h and cell death was analyzed by flow cytometric analysis of Annexin V/PI staining. Representative profiles and plotted apoptotic index are shown

FIG. 8 is a series of illustrations that show the upregulation of Noxa and Puma by (−)-gossypol in dose-dependent manner in 2LMP and in Bax/Bak knock-down 2LMP or PC-3 cells. 2LMP cells were treated with (−)-gossypol for 24 h (A) or the transfected 2LMP or PC-3 cells were treated for 18 h. Cell death was analyzed by trypan blue dye exclusion and whole cell lysates were analyzed by western blot.

FIG. 9 is a series of illustrations that show upregulation of Noxa and Puma proteins by (−)-gossypol. 2LMP and PC-3 cells were treated with (−)-gossypol at 10 μM for indicated time. Cell death was analyzed by trypan blue dye exclusion and whole cell lysates were analyzed by western blot.

FIG. 10 is a series of illustrations that show upregulation of Noxa and Puma proteins by (−)-gossypol in various breast cancer cell lines. Breast cancer lines were treated with (−)-gossypol for 24 h and cell death was analyzed by trypan blue dye exclusion and the whole cell lysates were analyzed by western blot.

FIG. 11 is a series of illustrations that show upregulation of Noxa and Puma proteins by (−)-gossypol in various prostate cancer cell lines. Prostate cancer cell lines were treated with (−)-gossypol for 24 h and cell death was analyzed by trypan blue dye exclusion and the whole cell lysates were analyzed by western blot

FIG. 12 is a series of panels, a line graph, and a series of illustrations that show the decrease of (−)-gossypol-induced apoptosis in Noxa knock-down 2LMP cells. 2LMP cells were transfected with SiRNAs of GFP or Noxa, then treated with (−)-gossypol for 18 h and analyzed for apoptosis. Three independent experiments were performed and representative profiles and plotted apoptotic indices are shown. Whole cell lysates were analyzed by western blot.

FIG. 13 is a series of panels, a line graph, and a series of illustrations that show the decrease of (−)-gossypol-induced apoptosis in Puma knock-down 2LMP cells. 2LMP cells were transfected with SiRNAs of GFP or Puma, then treated with (−)-gossypol for 18 h and analyzed for apoptosis. Three independent experiments were performed and representative profiles and plotted apoptotic indices are shown. Whole cell lysates were analyzed by western blot.

FIG. 14 is a series of illustrations that show (−)-gossypol-induced expression levels in Noxa knock-down 2LMP cells. 2LMP cells were transfected with SiRNAs of GFP or Noxa, then treated with (−)-gossypol for 18 h and analyzed for apoptosis. Whole cell lysates were analyzed by western blot.

FIG. 15 is a series of illustrations that show (−)-gossypol-induced expression levels in Puma knock-down 2LMP cells. 2LMP cells were transfected with SiRNAs of GFP or Puma, then treated with (−)-gossypol for 18 h and analyzed for apoptosis. Whole cell lysates were analyzed by western blot.

FIG. 16 is a series of panels and a line graph that show the decrease of (−)-gossypol-induced apoptosis in Noxa or Puma knock-down PC-3 cells. PC-3 cells were transfected with SiRNAs of GFP, Noxa or Puma, then treated with (−)-gossypol for 18 h and analyzed for apoptosis. Three independent experiments were performed and a representative profiles and plotted apoptotic indices are shown.

FIG. 17 is a series of illustrations that show (−)-gossypol-induced expression levels in Noxa knock-down PC-3 cells. PC-3 cells were transfected by SiRNAs of GFP or Noxa, then treated with (−)-gossypol for 18 h and analyzed for apoptosis. Whole cell lysate were analyzed by western blot.

FIG. 18 is a series of illustrations that show (−)-gossypol-induced expression levels in Puma knock-down PC-3 cells. PC-3 cells were transfected by SiRNAs of GFP or Puma, then treated with (−)-gossypol for 18 h and analyzed for apoptosis. Whole cell lysates were analyzed by western blot.

FIG. 19 is a line graph and a series of illustrations that show the selective induction of Noxa in tumor cells, but not in normal human primary cells or cell lines. 2LMP, MCF-12F and FF cells were treated with 10 μM of (−)-gossypol for the indicated times. Cell death was analyzed by trypan blue dye exclusion and whole cell lysates were analyzed by western blot.

FIG. 20 is a line graph and a series of illustrations that show the selective induction of Noxa in tumor cells, but not in normal human primary cells or cell lines. 2LMP and HMEC cells were treated with 10 μM of (−)-gossypol at the indicated time points. Cell death was analyzed by trypan blue dye exclusion and whole cell lysates were analyzed by western blot.

FIG. 21 a line graph and a series of illustrations that show the selective induction of Noxa in tumor cells, but not in normal human primary cells or cell lines. 2LMP, MCF-12F, FF cells were treated with (−)-gossypol for 24 h at the indicated doses. Cell death was analyzed by trypan blue dye exclusion and whole cell lysates were analyzed by western blot.

FIG. 22 is three bar graphs that show the transcriptional upregulation of Noxa and Puma by (−)-gossypol and the blockage of this upregulation by actinomycin D. 2LMP cells were treated with 10 μm of (−)-gossypol in the presence or the absence of actinomycin D (Act D) and a Taqman real-time PCR was performed. Data are shown as mean±SEM, error bars represent SEM.

FIG. 23 is three bar graphs that show the transcriptional upregulation of Noxa and Puma by (−)-gossypol and the blockage of this upregulation by actinomycin D. PC-3 cells were treated with 10 μm of (−)-gossypol in the presence or the absence of actinomycin D (Act D) and a Taqman real-time PCR was performed. Data are shown as mean±SEM, error bars represent SEM.

FIG. 24 is three bar graphs that show the transcriptional upregulation of Noxa and Puma by (−)-gossypol and the blockage of this upregulation by actinomycin D. MDA-MB-436 cells were treated with 10 μm of (−)-gossypol in the presence or the absence of actinomycin D (Act D) and a Taqman real-time PCR was performed. Data are shown as mean±SEM, error bars represent SEM.

FIG. 25 is three bar graphs that show the transcriptional upregulation of Noxa and Puma by (−)-gossypol and the blockage of this upregulation by actinomycin D. DU145 cells were treated with 10 μm of (−)-gossypol in the presence or the absence of actinomycin D (Act D) and a Taqman real-time PCR was performed. Data are shown as mean±SEM, error bars represent SEM.

FIG. 26 is a series of illustrations that show the blockage of (−)-gossypol-induced Noxa and Puma protein upregulation by Actinomycin D. 2LMP and PC-3 cells were treated with (−)-gossypol in the presence of Actinomycin D (Act D) at the indicated dose and time. The cells were harvested and whole cell lysates were analyzed by western blot.

FIG. 27 is a series of illustrations that show the transcriptional upregulation of Noxa and Puma by (−)-gossypol. 2LMP cells were transfected with SiRNAs of GFP, HIF-1α, and E2F1. After 24 h, cells were treated by (−)-gossypol for 18 h. Cell death was analyzed by trypan blue dye exclusion and whole cell lysate were analyzed by western blot.

FIG. 28 is a series of panels, a bar graph, and a series of illustrations that show the blockage of (−)-gossypol-induced apoptosis by c-Myc knock-down. SiRNAs of GFP or c-Myc were transfected in 2LMP cells and the cells were treated with (−)-gossypol and analyzed for apoptosis. Three independent experiments were performed and a representative profiles and plotted apoptotic index are shown. Whole cell lysates were analyzed by western blot.

FIG. 29 is a series of panels, a bar graph, and a series of illustrations that show the blockage of (−)-gossypol-induced apoptosis by c-Myc knock-down. SiRNAs of GFP or c-Myc were transfected in PC-3 cells and the cells were treated with (−)-gossypol and analyzed for apoptosis. Three independent experiments were performed and a representative profiles and plotted apoptotic index are shown. Whole cell lysates were analyzed by western blot.

FIG. 30 is two line graphs and a series of illustrations that show the comparison of the effect of c-Myc knock-down on the upregulation of Noxa by (−)-gossypol and Bortizomib. 2LMP cells were transfected with SiRNA of GFP or c-Myc, then treated with (−)-gossypol or Bortizomib for 24 h. Cell death was analyzed by trypan blue dye exclusion and whole cell lysate were analyzed by western blot.

FIG. 31 is two line graphs, a bar graph, and two illustrations that show the overcome of Mcl-1-mediated resistance to ABT-737-mediated cell death by (−)-gossypol. 2LMP (A) and PC -3 (B) cells were treated with (−)-gossypol and/or ABT-737 for 4 days. Cell viability was analyzed by WST assay. (C) 2LMP cells were treated with (−)-gossypol and/or ABT-737 for 48 h. Cell death was determined by trypan blue dye exclusion. (D) 2LMP cells were treated by 10 μm of (−)-gossypol or DMSO for 24 h. The capacity of Mcl-1 to bind Noxa was tested by coimmunoprecipitation assay using anti-Mcl-1 antibodies. Legend: (Control) immunoprecipitation without antibody; (pellet) Immunoprecipitation pellet; (supernatant) immunoprecipitation supernatant; (WCL) 10% of whole cell lysate; (D) DMSO; and (G) (−)-gossypol.

FIG. 32 is a line graph that shows the overcome of Mcl-1-mediated resistance to ABT-737-mediated apoptosis by (−)-gossypol. MDA-MB-436 cells were treated with (−)-gossypol and/or ABT-737 for 4 days. Cell viability was analyzed by WST assay.

FIG. 33 is a bar graph and a series of panels that show the overcome of Mcl-1-mediated resistance to ABT-737-mediated apoptosis by (−)-gossypol. 2LMP cells were treated with (−)-gossypol and/or ABT-737 for 48 h. Apoptosis was determined by flow cytometric analysis of Annexin V/PI staining Representative profiles and plotted apoptotic index are shown.

### DETAILED DESCRIPTION OF THE INVENTION

### Gossypol

The term “gossypol,” as used herein refers to racemic gossypol (i.e, (±)-gossypol), (−)-gossypol, or (+)-gossypol, and compositions thereof. In certain embodiments of the invention, the term “gossypol” refers to (−)-gossypol. In one such embodiment, the term “(−)-gossypol” refers to (−)-gossypol co-crystals. In another such embodiment, the term “(−)-gossypol” refers to (−)-gossypol acetic acid co-crystals in a ratio of about 1:1.

The term “(−)-gossypol,” as used herein refers to an optically active composition of gossypol wherein the active molecules comprising the composition rotate plane polarized light counterclockwise (e.g., levorotatory molecules) as measured by a polarimeter. Preferably, (−)-gossypol has an enantiomeric excess of 1% to 100%. In certain embodiments of the invention, (−)-gossypol has an enantiomeric excess of at least about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or about 100% (−)-gossypol. In one example, the specific rotation ([α]_{D}) of (−)-gossypol is about −350° to about −390°, about −375° to about −390°, or about −385° to about −390°. (See e.g., Dowd, Chirality, 15:486 (2003); Ciesielska et al., Chem. Phys. Lett. 353:69 (2992); Freedman et al., Chirality, 15:196 (2003); and Zhou et al., Kexue Tongbao, 28:1574 (1983)). Methods for resolving racemic gossypol compounds into substantially purified (+)- or (−)-gossypol are known (See, e.g., U.S. Published Application No. 2008/0021110).

Compositions comprising gossypol useful in the methods of this invention may be prepared using methods known to those of skill in the art. For example, the methods disclosed in U.S. Pat. No. 7,342,046 may be used to prepare a composition consisting essentially of co-crystals of (−)-gossypol with acetic acid in a molar ratio of 1:1.

Pharmaceutical compositions comprising gossypol may be produced by combining a therapeutically effective amount of gossypol with a pharmaceutically acceptable carrier. Pharmaceutical compositions may comprise, for example, (±)-gossypol, (+)-gossypol, (−)-gossypol, (±)-gossypol co-crystals, (−)-gossypol co-crystals, or (−)-gossypol acetic acid co-crystals. In certain embodiments of the invention, pharmaceutical compositions comprise (−)-gossypol or (−)-gossypol acetic acid co-crystals.

Pharmaceutical compositions useful within the scope of this invention include all compositions wherein gossypol is contained in an amount which is effective to achieve its intended purpose, e.g., to treat, ameliorate or prevent a disease, condition or disorder responsive to the induction of apoptosis. While individual needs vary, determination of optimal ranges of effective amounts of each component in a pharmaceutical composition is within the ordinary skill of a clinical practitioner in the art.

In certain embodiments of the invention, the pharmaceutical composition comprising gossypol is administered to patients, e.g., humans, orally at a dose totaling about 1 mg to about 1200 mg, or an equivalent dose of the pharmaceutically acceptable salt thereof, per day. In one such embodiment, a total oral dose of about 5 mg to about 500 mg, about 5 mg to about 250 mg, about 5 mg to about 100 mg, or about 5 mg to about 80 mg is administered per day. In another such embodiment, a total oral dose of about 90 mg to about 240 mg is administered per day. In still another such another embodiment, a total oral dose of about 80 mg to about 200 mg is administered per day.

In additional embodiments, an oral dose of about 5 mg, 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg or 200 mg is administered two times a day. In one such embodiment, an oral dose of 30 mg, 35 mg, 40 mg, 45 mg, 50 mg, 55 mg, 60 mg, 65 mg, 70 mg, 75 mg or 80 mg is administered two times a day. In another such embodiment, an oral dose of 40 mg, 45 mg, 50 mg, 55 mg or 60 mg, particularly 40 mg, is administered two times a day.

In another embodiment, an oral dose of about 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg or 400 mg is administered once a day. In another embodiment, an oral dose of 80 mg, 90 mg, 100 mg, 110 mg, 120 mg, 130 mg, 140 mg, 150 mg, 160 mg, 170 mg, 180 mg, 190 mg or 200 mg is administered once a day. In certain embodiments, the amount (e.g., dose) of gossypol administered may increase as the period of time between dosing increases, since the potential for adverse events may decrease under such circumstances.

The unit oral dose may comprise from about 1 mg to about 1000 mg of the pharmaceutical composition comprising gossypol. In one embodiment, the unit oral dose may comprise from about 5 mg to about 500 mg of the pharmaceutical composition comprising gossypol. In another embodiment, the unit oral dose may comprise from about 5 mg to about 100 mg. In another embodiment, the unit oral dose may comprise from about 5 mg to about 50 mg. The unit dose may be administered one or more times daily as one or more tablets, capsules and the like, each containing from about 1 to about 1000 mg of the pharmaceutical composition comprising gossypol, conveniently about 5 mg to about 100 mg of the composition, e.g., about 1 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg or 100 mg.

For intramuscular injection, the dose is generally about one-half of the oral dose. For example, a suitable intramuscular dose of gossypol would be about 0.5 mg to about 500 mg. In one embodiment, the intramuscular dose would be about 0.5 mg to about 100 mg, about 0.5 to about 50 mg, about 0.5 mg to about 25 mg, or about 0.5 mg to about 15 mg.

In a topical formulation, the composition may be present at a concentration of about 0.01 to 100 mg per gram of carrier. In one embodiment, the composition is present at a concentration of about 0.07 mg/g to about 1.0 mg/g, about 0.1 to about 0.5 mg/g, or about 0.4 mg/g.

Gossypol may be administered as part of a pharmaceutical composition containing suitable pharmaceutically acceptable carriers comprising excipients and auxiliaries which facilitate processing of the compositions into preparations which can be used pharmaceutically. Pharmaceutical preparations which can be administered orally include tablets, dragees, slow release lozenges, capsules and the like. Pharmaceutical preparations which can be administered topically include mouth rinses and mouth washes, gels, liquid suspensions, hair rinses, hair gels, shampoos and the like. Pharmaceutical preparations which can be administered rectally include suppositories and the like. Pharmaceutical preparations for administration by injection include suitable solutions and the like. Pharmaceutical preparations contain from about 0.01 to about 99 percent, or from about 0.25 to about 75 percent of gossypol, together with the excipient(s).

Gossypol and pharmaceutical compositions thereof may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, if any, frequency of treatment, and the nature of the effect desired.

The pharmaceutical preparations used in the methods of the present invention are manufactured in a manner which is itself known, for example, by means of conventional mixing, granulating, dragee-making, dissolving, or lyophilizing processes. Thus, pharmaceutical preparations for oral use can be obtained by combining the active compounds with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate, are used. Dye stuffs or pigments may be added to the tablets or dragee coatings, for example, for identification or in order to characterize combinations of active compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which may be mixed with fillers such as lactose, binders such as starches, and/or lubricants such as talc or magnesium stearate and, optionally, stabilizers. In soft capsules, the active compounds are preferably dissolved or suspended in suitable liquids, such as fatty oils, or liquid paraffin. In addition, stabilizers may be added.

Possible pharmaceutical preparations which can be used rectally include, for example, suppositories, which consist of a combination of one or more of the active compounds with a suppository base. Suitable suppository bases are, for example, natural or synthetic triglycerides, or paraffin hydrocarbons. In addition, it is also possible to use gelatin rectal capsules which consist of a combination of the active compounds with a base. Possible base materials include, for example, liquid triglycerides, polyethylene glycols, or paraffin hydrocarbons.

Suitable formulations for parenteral administration include aqueous solutions of the active compounds in water-soluble form, for example, water-soluble salts and alkaline solutions. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides or polyethylene glycol-400. Aqueous injection suspensions may contain substances which increase the viscosity of the suspension include, for example, sodium carboxymethyl cellulose, sorbitol, and/or dextran. Optionally, the suspension may also contain stabilizers.

The topical compositions of this invention are formulated preferably as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than C₁₂). The preferred carriers are those in which the active ingredient is soluble. Emulsifiers, stabilizers, humectants and antioxidants may also be included as well as agents imparting color or fragrance, if desired. Additionally, transdermal penetration enhancers can be employed in these topical formulations. Examples of such enhancers can be found in U.S. Pat. Nos. 3,989,816 and 4,444,762.

Creams are preferably formulated from a mixture of mineral oil, self-emulsifying beeswax and water in which mixture the active ingredient, dissolved in a small amount of an oil such as almond oil, is admixed. A typical example of such a cream is one which includes about 40 parts water, about 20 parts beeswax, about 40 parts mineral oil and about 1 part almond oil.

Ointments may be formulated by mixing a suspension of the active ingredient in a vegetable oil such as almond oil with warm soft paraffin and allowing the mixture to cool. A typical example of such an ointment is one which includes about 30% almond oil and about 70% white soft paraffin by weight.

Lotions may be conveniently prepared by preparing a suspension of the active ingredient in a suitable high molecular weight alcohol such as propylene glycol or polyethylene glycol.

The term “gossypol co-crystal,” as used herein, refers to a composition comprising co-crystals of (±)-gossypol and a C₁₋₈ carboxylic acid, C₁₋₈ sulfonic acid, C₁₋₁₂ ketone or C₁₋₁₂ diketone.

The term “C₁₋₈ carboxylic acid,” as used herein, refers to straight-chained or branched, aromatic or non-aromatic, saturated or unsaturated, substituted or unsubstituted C₁₋₈ carboxylic acid, including, but not limited to, formic acid, acetic acid, propionic acid, n-butyric acid, t-butyric acid, n-pentanoic acid, 2-pentanoic acid, n-hexanoic acid, 2-hexanoic acid, n-heptanoic acid, n-octanoic acid, acrylic acid, succinic acid, fumaric acid, malic acid, tartaric acid, citric acid, lactic acid, and benzoic acid.

The term “C₁₋₈ sulfonic acid,” as used herein, refers to straight-chained or branched, aromatic or non-aromatic, saturated or unsaturated, substituted or unsubstituted C₁₋₈ sulfonic acid, including, but not limited to, methanesulfonic acid, ethanesulfonic acid, n-propanesulfonic acid, 2-propanesulfonic acid, n-butanesulfonic acid, n-pentanesulfonic acid n-hexanesulfonic acid, n-heptanesulfonic acid, n-octanesulfonic acid, and benzenesulfonic acid.

The term “C₁₋₁₂ ketone,” as used herein, refers to straight-chained, cyclic or branched, aromatic or non-aromatic, saturated or unsaturated, substituted or unsubstituted C₁₋₁₂ ketone, including, but not limited to, acetone and cyclododecanone.

The term “C₁₋₁₂ diketone,” as used herein, refers to straight-chained or branched, aromatic or non-aromatic, saturated or unsaturated, substituted or unsubstituted C₁₋₁₂ diketone, including, but not limited to, 2,4-pentanedione.

The term “(−)-gossypol co-crystal,” as used herein, refers to a composition comprising (−)-gossypol and acetic acid, (−)-gossypol and acetone, (−)-gossypol and 2,4-pentanedione, or (−)-gossypol and cyclododecanone. In particular embodiments of the invention, the term “(−)-gossypol co-crystal,” refers to a composition comprising (−)-gossypol and acetic acid, referred herein as “(−)-gossypol acetic acid co-crystals” (see U.S. Pat. No. 7,342,046).

### DEFINITIONS

The term “biomarker” as used herein refers to any biological compound, such as a protein, a fragment of a protein, a peptide, a polypeptide, a nucleic acid, etc. that can be detected and quantified in a patient in vivo or that is present in a biological sample and that may be isolated from, or measured in, the biological sample. The expression levels of the biomarker can be measured at the protein or RNA (e.g., mRNA) levels. Furthermore, a biomarker can be the entire intact molecule, or it can be a portion thereof that may be partially functional or recognized, for example, by an antibody or other specific binding protein. In certain circumstances, a biomarker is considered to be informative if a measurable aspect of the biomarker is associated with a given state of the patient, such as a particular stage of hyperproliferative disease, such as cancer. Such a measurable aspect may include, for example, the presence, absence, or concentration (i.e., expression level) of the biomarker in a patient. In one particular embodiment of the invention, the measurable aspect of the biomarker is the expression level of the protein.

Thus, in certain aspects of the present invention, a biomarker is a biological compound (e.g., a protein such as c-Myc or Mcl-1) which is differentially present in a in a subject of one phenotypic status (e.g., a patient having a condition or disease such as cancer) as compared with another phenotypic status (e.g., a normal undiseased patient). A biomarker is differentially present between different phenotypic statuses if the mean or median expression level of the biomarker in the different groups is calculated to be statistically significant. Common tests for statistical significance include, among others, t-test, ANOVA, Kruskal-Wallis, Wilcoxon, Mann-Whitney, Significance Analysis of Microarrays, odds ratio, etc. Biomarkers, alone or in combination, provide measures of relative likelihood that a subject belongs to one phenotypic status or another. Therefore, they are useful, inter alia, as markers for disease and as indicators that particular therapeutic treatment regimens will likely result in beneficial patient outcomes.

In addition to individual biological compounds (e.g., c-Myc, Mcl-1 or Noxa), the term “biomarker” as used herein is meant to include groups or sets of multiple biological compounds. For example, the proteins c-Myc and Mcl-1 may comprise a biomarker. Thus, a “biomarker” may comprise one, two, three, four, five, six, seven, eight, nine, ten, fifteen, twenty, twenty five, thirty, or more, biological compounds.

The determination of the expression level of a biomarker, particularly a member of the Myc family of oncogene products, more particularly c-Myc, in a patient can be performed using any of the many methods known in the art. Any method known in the art for quantitating specific proteins such as c-Myc in a patient or a biological sample may be used in the methods of the invention. Examples include, but are not limited to, RT-PCR (real time PCR), Northern blot, Western blot, ELISA (enzyme linked immunosorbent assay), RIA (radioimmunoassay), gene chip analysis of RNA expression, immunohistochemistry or immunofluorescence (See, e.g., Slagle et al. Cancer 83:1401 (1998)). Certain embodiments of the invention include methods wherein biomarker RNA expression (transcription) is determined. Other embodiments of the invention include methods wherein protein expression in the biological sample is determined. See, for example, Harlow et al., Antibodies: A Laboratory Manual, Cold Spring Harbor Laboratory, Cold Spring Harbor, N.Y., (1988) and Ausubel et al., Current Protocols in Molecular Biology, John Wiley & Sons, New York 3rd Edition, (1995). When quantified in a patient in vivo, the expression level of proteins such as c-Myc may be determined by administering an antibody that binds specifically to c-Myc (See, e.g., U.S. Published Appl. No. 2006/0127945) and determining the extent of binding. The antibody may be detectably labeled, e.g., with a radioisotope such as carbon-11, nitrogen-13, oxygen-15, and fluorine-18. The label may then be detected by positron emission tomography (PET).

In one embodiment of the invention, a tumor tissue biopsy is obtained and the cells in the tumor tissue are assayed for determination of biomarker expression. In one such embodiment, the biomarker is an elevated expression level of c-Myc, Mcl-1, or c-Myc and Mcl-1 relative to the normal expression level of c-Myc, Mcl-1, or c-Myc and Mcl-1. In a particular embodiment, the biomarker is an elevated expression level of c-Myc relative to the normal expression level of c-Myc. For northern blot or RT-PCR analysis, RNA should be isolated from the tumor tissue sample using RNAse free techniques. Such techniques are commonly known in the art.

In one embodiment of the invention, PET imaging is used to determine biomarker expression.

In another embodiment of the invention, Northern blot analysis of biomarker transcription in a tumor cell sample is performed. Northern analysis is a standard method for detection and quantitation of mRNA levels in a sample. Initially, RNA is isolated from a sample to be assayed using Northern blot analysis. In the analysis, the RNA samples are first separated by size via electrophoresis in an agarose gel under denaturing conditions. The RNA is then transferred to a membrane, crosslinked and hybridized with a labeled probe. Typically, Northern hybridization involves polymerizing radiolabeled or nonisotopically labeled DNA, in vitro, or generation of oligonucleotides as hybridization probes. Typically, the membrane holding the RNA sample is prehybridized or blocked prior to probe hybridization to prevent the probe from coating the membrane and, thus, to reduce non-specific background signal. After hybridization, typically, unhybridized probe is removed by washing in several changes of buffer. Stringency of the wash and hybridization conditions can be designed, selected and implemented by any practitioner of ordinary skill in the art. If a radiolabeled probe was used, the blot can be wrapped in plastic wrap to keep it from drying out and then immediately exposed to film for autoradiography. If a nonisotopic probe was used, the blot must generally be treated with nonisotopic detection reagents prior to film exposure. The relative levels of expression of the genes being assayed can be quantified using, for example, densitometry.

In another embodiment of the invention, biomarker expression is determined using RT-PCR. RT-PCR allows detection of the progress of a PCR amplification of a target gene in real time. Design of the primers and probes required to detect expression of a biomarker of the invention is within the skill of a practitioner of ordinary skill in the art. RT-PCR can be used to determine the level of RNA encoding a biomarker of the invention in a tumor tissue sample. In an embodiment of the invention, RNA from the tissue sample is isolated, under RNAse free conditions, than converted to DNA by treatment with reverse transcriptase. Methods for reverse transcriptase conversion of RNA to DNA are well known in the art. A description of PCR is provided in the following references: Mullis et al., Cold Spring Harbor Symp. Quant. Biol. 51:263 (1986); EP 50,424; EP 84,796; EP 258,017; EP 237,362; EP 201,184; U.S. Pat. Nos. 4,683,202; 4,582,788; 4,683,194.

RT-PCR probes depend on the 5′-3′ nuclease activity of the DNA polymerase used for PCR to hydrolyze an oligonucleotide that is hybridized to the target amplicon (biomarker gene). RT-PCR probes are oligonucleotides that have a fluorescent reporter dye attached to the 5, end and a quencher moiety coupled to the 3′ end (or vice versa). These probes are designed to hybridize to an internal region of a PCR product. In the unhybridized state, the proximity of the fluor and the quench molecules prevents the detection of fluorescent signal from the probe. During PCR amplification, when the polymerase replicates a template on which an RT-PCR probe is bound, the 5′-3′ nuclease activity of the polymerase cleaves the probe. This decouples the fluorescent and quenching dyes and FRET no longer occurs. Thus, fluorescence increases in each cycle, in a manner proportional to the amount of probe cleavage. Fluorescence signal emitted from the reaction can be measured or followed over time using equipment which is commercially available using routine and conventional techniques.

In still another embodiment of the invention, expression of proteins encoded by biomarkers are detected by western blot analysis. A western blot (also known as an immunoblot) is a method for protein detection in a given sample of tissue homogenate or extract. It uses gel electrophoresis to separate denatured proteins by mass. The proteins are then transferred out of the gel and onto a membrane (e.g., nitrocellulose or polyvinylidene fluoride (PVDF)), where they are “probed” using antibodies specific to the protein. Antibodies that recognize a protein in a band on the membrane will bind to it. The bound antibodies are then bound by a secondary anti-antibody antibody which is conjugated with a detectable label (e.g., biotin, horseradish peroxidase or alkaline phosphatase). Detection of the secondary label signal indicates the presence of the protein.

In still another embodiment of the invention, the expression of a protein encoded by a biomarker is detected by enzyme-linked immunosorbent assay (ELISA). In one embodiment of the invention, “sandwich ELISA” comprises coating a plate with a capture antibody; adding sample wherein any antigen present binds to the capture antibody; adding a detecting antibody which also binds the antigen; adding an enzyme-linked secondary antibody which binds to detecting antibody; and adding substrate which is converted by an enzyme on the secondary antibody to a detectable form. Detection of the signal from the secondary antibody indicates presence of the biomarker antigen protein.

In still another embodiment of the invention, the expression of a biomarker is evaluated by use of a gene chip or microarray. Such techniques are within ordinary skill held in the art.

The term “biological sample” as used herein refers any tissue or fluid from a subject that is suitable for detecting a biomarker, particularly the expression level c-Myc. Examples of useful biological samples include, but are not limited to, biopsied tissues, e.g., solid tumor, lymph gland, inflamed tissue, tissue involved in a condition or disease, blood, plasma, serous fluid, cerebrospinal fluid, saliva, urine, lymph, cerebral spinal fluid, and the like. Other suitable biological samples will be familiar to those of ordinary skill in the relevant arts. A biological sample can be analyzed for biomarker expression using any technique known in the art and can be obtained using techniques that are well within the scope of ordinary knowledge of a clinical practitioner.

The term “elevated expression level,” as used herein, refers to the level of a biomarker (e.g., c-Myc protein or c-Myc mRNA) that is at least about 5% greater than the normal expression level of said biomarker. Thus, in certain embodiments, the elevated expression level of a biomarker is at least about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% greater, or more, than the normal expression level of said biomarker.

The term “normal expression level” as used herein refers to either the expression level of a biomarker in the adjacent tissue around the tissue involved in the disorder or the standard expression level of the biomarker from a biological sample in a healthy subjects. The standard expression level of a biomarker in healthy subjects may represent the average of a suitable number of members of the general population, e.g., at least about 10, at least about 50, at least about 100, or more than 100 members of the general population. In one embodiment, the standard level in healthy subjects is determined in an age-matched fashion, e.g., the subject on whom the methods of the invention are being practiced is compared to healthy subjects of the same age.

The term “therapeutic agent,” as used herein refers to any chemical substance or surgical technique that can be used in the treatment, management, or amelioration of a disease, condition or disorder or one or more symptoms thereof. Suitable therapeutic agents include, but are not limited to, small molecules, synthetic drugs, peptides, polypeptides, proteins, nucleic acids (e.g., DNA and RNA polynucleotides including, but not limited to, antisense nucleotide sequences, triple helices, and nucleotide sequences encoding biologically active proteins, polypeptides, or peptides), antibodies, synthetic or natural inorganic molecules, mimetic agents, and synthetic or natural organic molecules. Typically the therapeutic agent is one which is known to be useful for, or has been, or is currently being used for the treatment, management, prevention, or amelioration of a condition or disorder or one or more symptoms thereof.

Antibodies useful as therapeutic agents include, but are not limited to, monoclonal antibodies, synthetic antibodies, multispecific antibodies (including bi-specific antibodies), human antibodies, humanized antibodies, chimeric antibodies, single-chain Fvs (scFv) (including bi-specific scFvs), single chain antibodies, Fab fragments, F(ab′) fragments, disulfide-linked Fvs (sdFv), and epitope-binding fragments of any of the above. In particular, antibodies include immunoglobulin molecules and immunologically active portions of immunoglobulin molecules, i.e., molecules that contain an antigen binding site that immunospecifically binds to an antigen. The immunoglobulin molecules of the invention can be of any type (e.g., IgG, IgE, IgM, IgD, IgA and IgY), class (e.g., IgG₁, IgG₂, IgG₃, IgG₄, IgA₁ and IgA₂) or subclass of immunoglobulin molecule.

In particular non-limiting embodiments, the antibody is Rituxan™ (useful for treating non-Hodgkin's lymphoma), Herceptin™ (useful for treating metastatic breast cancer), Campath™ (useful for treating chronic lymphocytic leukemia), Erbitux™ (useful for treating various cancers), MDX-010 (useful for treating malignant melanoma, prostate cancer), MDX-214 (useful for treatment of cancer), AlloMune™ (useful for treating non-Hodgkin's lymphoma, Hodgkin's disease), IMC-255 (antibody to epidermal growth factor), A7-neocarzinostatin (useful for the treatment of liver metastasis), 791T/36 (useful for the treatment of colorectal cancer), Fas/APO-1 (useful for treatment of malignant glioma cells), doxorubicin-CLNIgG (useful for the treatment of malignant glioma cells), siplizumab (useful for the treatment of T-cell lymphoma), Vitaxin™ (an antiangiogenic antibody useful for the treatment of cancer), MT-103 (useful for the treatment of Hodgkin's lymphoma), Orthoclone™ (useful for treating heart, liver and kidney transplant rejection), ReoPro™ (useful for reducing post-cardiovascular-surgery clotting), Remicade™ (useful for treating Crohn's disease, rheumatoid arthritis), ABX-CBL (useful for preventing transplant rejection), AD-439 (useful for treating HIV infection), or SB-240563 (useful for treatment of asthma, allergies).

Other therapeutic agents useful in the methods of the invention include, but are not limited to, vasodilators (e.g., nitrates, calcium channel blockers), anticoagulants (e.g., heparin), anti-platelet agents (e.g., aspirin, blockers of IIb/IIIa receptors, clopidogrel), anti-thrombins (e.g., hirudin, iloprost), immunosuppressants (e.g., sirolimus, tranilast, dexamethasone, tacrolimus, everolimus, A24), collagen synthetase inhibitors (e.g., halofuginone, propyl hydroxylase, C-proteinase inhibitor, metalloproteinase inhibitor), anti-inflammatories (e.g., corticosteroids such as alclometasone, amcinonide, betamethasone, beclomethasone, budesonide, cortisone, clobetasol, clocortolone, desonide, dexamethasone, desoximetasone, diflorasone, flunisolide, fluticasone, fluocinonide, flurandrenolide, halcinonide, hydrocortisone, methylprednisolone, mometasone, prednicarbate, prednisone, prednisolone and triamcinolone; non-steroidal anti-inflammatory drugs), 17β-estradiol, angiotensin converting enzyme inhibitors, colchicine, fibroblast growth factor antagonists, histamine antagonists, lovastatin, nitroprusside, phosphodiesterase inhibitors, prostaglandin inhibitors, suramin, serotonin blockers, thioprotease inhibitors, platelet-derived growth factor antagonists, nitric oxide, and angiopeptin.

Anti-inflammatory drugs useful in the methods of the invention include, but are not limited to, salicylates (such as aspirin, choline magnesium trisalicylate, methyl salicylate, salsalte and diflunisal), acetic acids (such as indomethacin, sulindac, tolmetin, aceclofenac and diclofenac), 2-arylpropionic acids or profens (such as ibuprofen, ketoprofen, naproxen, fenoprofen, flurbiprofen and oxaprozin), N-arylanthranilic acids or fenamic acids (such as mefenamic acid, flufenamic acid, and meclofenamate), enolic acids or oxicams (such as piroxicam and meloxicam), cox inhibitors (such as celecoxib, rofecoxib (withdrawn from market), valdecoxib, parecoxib and etoricoxib), sulphonanilides such as nimesulide; naphthylalkanones (such as nabumetone), pyranocarboxylic acids (such as etodolac) and pyrroles (such as ketorolac).

A particularly useful immunomodulatory agent useful in the methods of the invention includes, but is not limited to, thalidomide.

Immunosuppressant agents are useful to counteract autoimmune diseases, such as rheumatoid arthritis or Crohn's disease, and to prevent the immune system from attacking healthy parts of the body. Immunosuppressive agents useful in the methods of the invention include, but are not limited to, glucocorticoid receptor agonists (e.g., cortisone, dexamethasone, hydrocortisone, betamethasone), calcineurin inhibitors (e.g., macrolides such as tacrolimus and pimecrolimus), immunophilins (e.g., cyclosporin A) and mTOR inhibitors (e.g., sirolimus, marketed as RAPAMUNE® by Wyeth). In other embodiments, immunomodulatory agents useful for the present invention further include antiproliferative agents (e.g., methotrexate, leflunomide, cisplatin, ifosfamide, paclitaxel, taxanes, topoisomerase I inhibitors (e.g., CPT-11, topotecan, 9-AC, and GG-211), gemcitabine, vinorelbine, oxaliplatin, 5-fluorouracil (5-FU), leucovorin, vinorelbine, temodal, taxol, cytochalasin B, gramicidin D, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, melphalan, glucocorticoids, procaine, tetracaine, lidocaine, propranolol, puromycin homologs, and cytoxan.

Immunostimulant agents are useful to increase the efficiency of the immune system and treat immunodeficiency disorders. Immunostimulant agents useful in the methods of the invention include, but are not limited to, interferon and Zidovudine (AZT).

Antimicrobial agents for use in the methods of the invention may include any agent that can kill, inhibit, or otherwise attenuate the function of microbial organisms, as well as any agent contemplated to have such activities. Antimicrobial agent useful in the methods of the invention include, but are not limited to, natural and synthetic antibiotics, antibodies, inhibitory proteins (e.g., defensins), antisense nucleic acids, membrane disruptive agents and the like, used alone or in combination. Indeed, any type of antibiotic may be used including, but not limited to, antibacterial agents, antiviral agents, antifungal agents, and the like.

In particular embodiments of the invention, the therapeutic agent is an anticancer agent. In one such embodiment, the anticancer agent is a chemotherapeutic agent, e.g., a taxane, such as but not limited to, docetaxel or paclitaxel. In another such embodiment, the anticancer is a radiotherapeutic agent.

The term “anticancer agent,” as used herein refers to any therapeutic agent known to the clinical practitioner of ordinary skill in the art (e.g., chemotherapeutic agent, radiotherapeutic agent, surgical intervention, etc.) used in the treatment or amelioration of cancer and/or as an inducer of apoptosis in a patient (e.g., in mammals, particularly humans).

The term “chemotherapeutic agent,” as used herein refers to any chemical substance known to the clinical practitioner of ordinary skill in the art used for the treatment or amelioration of cancer and/or as an inducer of apoptosis in a patient. Suitable chemotherapeutic agents include, but are not limited to, abraxane, actinomycin D, aldesleukin, alemtuzumab, alitretinoin, allopurinol, altretamine, amifostine, aminoglutethamide, anastrozole, arsenic trioxide, asparaginase, azacitidine, azathioprine, BCG live, bevacizumab, bexarotene, bicalutamide, bleomycin, bortezomib, busulfan, butazolidin, capecitabine, carboplatin, carmustine, celecoxib, chlorambucil, cisplatin, cladribine, clofarabine, cyclophosphamide, cytarabine, dacarbazine, dactinomycin, darbepoetin alfa, daunomycin, daunorubicin, denileukin diftitox, dexamethasone, dexrazoxane, diethylstilbestrol, docetaxel, doxorubicin, dromostanolone propionate, epirubicin, epoetin alfa, estramustine, ethinyl estradiol, etoposide, exemestane, filgrastim, finasteride, floxuridine, fludarabine, fluorouracil, fluoxymesterone, flutamide, fulvestrant, gefitinib, gemcitabine, gemtuzumab, goserelin acetate, hexamethylmelamine, hydroxychloroquine, hydroxyprogesterone caproate, hydroxyurea, ibritumomab, idarubicin, ifosfamide, imatinib, interferon alfa-2a, interferon alfa-2b, interleukin-2, irinotecan, ketoconazole, letrozole, leucovorin, leuprolide, levamisole HCl, lomustine, mechlorethamine, medroxyprogesterone acetate, megestrol acetate, meloxicam, melphalan, mercaptopurine, mesna, methotrexate, methoxsalen, methylprednisolone, metronidazole, misonidazole, mithramycin, mitomycin, mitotane, mitoxantrone, nandrolone phenpropionate, nitrogen mustard, nitroimidazole, nitrosourea, nofetumomab, oblimersen sodium, oprelvekin, oxaliplatin, oxaliplatin, oxyphenbutazone, paclitaxel, pamidronate, pegademase, pegaspargase, pegfilgrastim, pentostatin, phenylbutazone, picoplatin, pipobroman, plicamycin, plicamycin, porfimer sodium, prednisolone, prednisone, procarbazine, procarbazine, quinacrine, raloxifene, rasburicase, rituximab, romidepsin, sargramostim, semustine, streptozocin, talc, tamoxifen, temozolomide, teniposide, testolactone, testosterone propionate, thalidomide, thioguanine, thiotepa, tiripazamine, topotecan HCl, toremifene, tositumomab, trastuzumab, tretinoin, trimethoprim/sulfamethoxazole, uracil mustard, valrubicin, vinblastine, vincristine, vindesine, vinorelbine, and zoledronic acid.

Any oncolytic agent that is used in a chemotherapy context finds use in the methods of the present invention. For example, the U.S. Food and Drug Administration (U.S.F.D.A.) maintains a formulary of oncolytic agents approved for use in the United States. International counterpart agencies to the U.S.F.D.A. maintain similar formularies. Table 1 provides a list of exemplary chemotherapeutic agents approved for use in the U.S. Those skilled in the art will appreciate that the “product labels” required on all U.S. approved chemotherapeutics describe approved indications, dosing information, toxicity data, and the like, for the exemplary agents.

TABLE 1

| | | |
|---|---|---|
| Aldesleukin | Proleukin | Chiron Corp., |
| (des-alanyl-1, serine-125 human | | Emeryville, CA |
| interleukin-2) | | |
| Alemtuzumab | Campath | Millennium and |
| (IgG1κ anti CD52 antibody) | | ILEX Partners, |
| | | LP, Cambridge, |
| | | MA |
| Alitretinoin | Panretin | Ligand |
| (9-cis-retinoic acid) | | Pharmaceuticals, |
| | | Inc., San Diego |
| | | CA |
| Allopurinol | Zyloprim | GlaxoSmithKline, |
| (1,5-dihydro-4H-pyrazolo[3,4- | | Research Triangle |
| d]pyrimidin-4-one monosodium salt) | | Park, NC |
| Altretamine | Hexalen | US Bioscience, |
| (N,N,N′,N′,N″,N″,-hexamethyl-1,3,5- | | West |
| triazine-2,4,6-triamine) | | Conshohocken, |
| | | PA |
| Amifostine | Ethyol | US Bioscience |
| (ethanethiol, 2-[(3-aminopropyl)amino]-, | | |
| dihydrogen phosphate (ester)) | | |
| Anastrozole | Arimidex | AstraZeneca |
| (1,3-Benzenediacetonitrile, a,a,a′,a′- | | Pharmaceuticals, |
| tetramethyl-5-(1H-1,2,4-triazol-1- | | LP, Wilmington, |
| ylmethyl)) | | DE |
| Arsenic trioxide | Trisenox | Cell Therapeutic, |
| | | Inc., Seattle, WA |
| Asparaginase | Elspar | Merck & Co., |
| (L-asparagine amidohydrolase, type EC-2) | | Inc., Whitehouse |
| | | Station, NJ |
| BCG Live | TICE BCG | Organon Teknika, |
| (lyophilized preparation of an attenuated | | Corp., Durham, |
| strain of Mycobacterium bovis (Bacillus | | NC |
| Calmette-Gukin [BCG], substrain | | |
| Montreal) | | |
| bexarotene capsules | Targretin | Ligand |
| (4-[1-(5,6,7,8-tetrahydro-3,5,5,8,8- | | Pharmaceuticals |
| pentamethyl-2-napthalenyl) ethenyl] | | |
| benzoic acid) | | |
| bexarotene gel | Targretin | Ligand |
| | | Pharmaceuticals |
| Bleomycin | Blenoxane | Bristol-Myers |
| (cytotoxic glycopeptide antibiotics | | Squibb Co., NY, |
| produced by Streptomyces verticillus; | | NY |
| bleomycin A₂ and bleomycin B₂) | | |
| Capecitabine | Xeloda | Roche |
| (5′-deoxy-5-fluoro-N- | | |
| [(pentyloxy)carbonyl]-cytidine) | | |
| Carboplatin | Paraplatin | Bristol-Myers |
| (platinum, diammine [1,1- | | Squibb |
| cyclobutanedicarboxylato(2-)-0,0′]-,(SP- | | |
| 4-2)) | | |
| Carmustine | BCNU, | Bristol-Myers |
| (1,3-bis(2-chloroethyl)-1-nitrosourea) | BiCNU | Squibb |
| Carmustine with Polifeprosan 20 Implant | Gliadel Wafer | Guilford |
| | | Pharmaceuticals, |
| | | Inc., Baltimore, |
| | | MD |
| Celecoxib | Celebrex | Searle |
| (as 4-[5-(4-methylphenyl)-3- | | Pharmaceuticals, |
| (trifluoromethyl)-1H-pyrazol-1-yl] | | England |
| benzenesulfonamide) | | |
| Chlorambucil | Leukeran | GlaxoSmithKline |
| (4- | | |
| [bis(2chlorethyl)amino]benzenebutanoic | | |
| acid) | | |
| Cisplatin | Platinol | Bristol-Myers |
| (PtCl₂H₆N₂) | | Squibb |
| Cladribine | Leustatin, 2- | R.W. Johnson |
| (2-chloro-2′-deoxy-b-D-adenosine) | CdA | Pharmaceutical |
| | | Research Institute, |
| | | Raritan, NJ |
| Cyclophosphamide | Cytoxan, | Bristol-Myers |
| (2-[bis(2-chloroethyl)amino] tetrahydro- | Neosar | Squibb |
| 2H-13,2-oxazaphosphorine 2-oxide | | |
| monohydrate) | | |
| Cytarabine | Cytosar-U | Pharmacia & |
| (1-b-D-Arabinofuranosylcytosine, | | Upjohn Company |
| C₉H₁₃N₃O₅) | | |
| cytarabine liposomal | DepoCyt | Skye |
| | | Pharmaceuticals, |
| | | Inc., San Diego, |
| | | CA |
| Dacarbazine | DTIC-Dome | Bayer AG, |
| (5-(3,3-dimethyl-l-triazeno)-imidazole-4- | | Leverkusen, |
| carboxamide (DTIC)) | | Germany |
| Dactinomycin, actinomycin D | Cosmegen | Merck |
| (actinomycin produced by Streptomyces | | |
| parvullus, C₆₂H₈₆N₁₂O₁₆) | | |
| Darbepoetin alfa | Aranesp | Amgen, Inc., |
| (recombinant peptide) | | Thousand Oaks, |
| | | CA |
| daunorubicin liposomal | DanuoXome | Nexstar |
| ((8S-cis)-8-acetyl-10-[(3-amino-2,3,6- | | Pharmaceuticals, |
| trideoxy-a-L-lyxo-hexopyranosyl)oxy]- | | Inc., Boulder, CO |
| 7,8,9,10-tetrahydro-6,8,11-trihydroxy-1- | | |
| methoxy-5,12-naphthacenedione | | |
| hydrochloride) | | |
| Daunorubicin HCl, daunomycin | Cerubidine | Wyeth Ayerst, |
| ((1S,3S)-3-Acetyl-1,2,3,4,6,11- | | Madison, NJ |
| hexahydro-3,5,12-trihydroxy-10-methoxy- | | |
| 6,11-dioxo-1-naphthacenyl 3-amino-2,3,6- | | |
| trideoxy-(alpha)-L-lyxo-hexopyranoside | | |
| hydrochloride) | | |
| Denileukin diftitox | Ontak | Seragen, Inc., |
| (recombinant peptide) | | Hopkinton, MA |
| Dexrazoxane | Zinecard | Pharmacia & |
| ((S)-4,4′-(1-methyl-1,2-ethanediyl)bis-2,6- | | Upjohn Company |
| piperazinedione) | | |
| Docetaxel | Taxotere | Aventis |
| ((2R,3S)-N-carboxy-3-phenylisoserine, N- | | Pharmaceuticals, |
| tert-butyl ester, 13-ester with 5b-20- | | Inc., Bridgewater, |
| epoxy-12a,4,7b,10b,13a-hexahydroxytax- | | NJ |
| 11-en-9-one 4-acetate 2-benzoate, | | |
| trihydrate) | | |
| Doxorubicin HCl | Adriamycin, | Pharmacia & |
| (8S,10S)-10-[(3-amino-2,3,6-trideoxy-a- | Rubex | Upjohn Company |
| L-lyxo-hexopyranosyl)oxy]-8-glycolyl- | | |
| 7,8,9,10-tetrahydro-6,8,11-trihydroxy-1- | | |
| methoxy-5,12-naphthacenedione | | |
| hydrochloride) | | |
| doxorubicin | Adriamycin | Pharmacia & |
| | PFS | Upjohn Company |
| | Intravenous | |
| | injection | |
| doxorubicin liposomal | Doxil | Sequus |
| | | Pharmaceuticals, |
| | | Inc., Menlo park, |
| | | CA |
| dromostanolone propionate | Dromostanolone | Eli Lilly & |
| (17b-Hydroxy-2a-methyl-5a-androstan-3- | | Company, |
| one propionate) | | Indianapolis, IN |
| dromostanolone propionate | Masterone | Syntex, Corp., |
| | injection | Palo Alto, CA |
| Elliott's B Solution | Elliott's B | Orphan Medical, |
| | Solution | Inc |
| Epirubicin | Ellence | Pharmacia & |
| ((8S-cis)-10-[(3-amino-2,3,6-trideoxy-a- | | Upjohn Company |
| L-arabino-hexopyranosyl)oxy]-7,8,9,10- | | |
| tetrahydro-6,8,11-trihydroxy-8- | | |
| (hydroxyacetyl)-1-methoxy-5,12- | | |
| naphthacenedione hydrochloride) | | |
| Epoetin alfa | Epogen | Amgen, Inc |
| (recombinant peptide) | | |
| Estramustine | Emcyt | Pharmacia & |
| (estra-1,3,5(10)-triene-3,17-diol(17(beta))-, | | Upjohn Company |
| 3-[bis(2-chloroethyl)carbamate] 17- | | |
| (dihydrogen phosphate), disodium salt, | | |
| monohydrate, or estradiol 3-[bis(2- | | |
| chloroethyl)carbamate] 17-(dihydrogen | | |
| phosphate), disodium salt, monohydrate) | | |
| Etoposide phosphate | Etopophos | Bristol-Myers |
| (4′-Demethylepipodophyllotoxin 9-[4,6-O-(R)- | | Squibb |
| ethylidene-(beta)-D-glucopyranoside], | | |
| 4′-(dihydrogen phosphate)) | | |
| etoposide, VP-16 | Vepesid | Bristol-Myers |
| (4′-demethylepipodophyllotoxin 9-[4,6-0- | | Squibb |
| (R)-ethylidene-(beta)-D-glucopyranoside]) | | |
| Exemestane | Aromasin | Pharmacia & |
| (6-methylenandrosta-1,4-diene-3,17- | | Upjohn Company |
| dione) | | |
| Filgrastim | Neupogen | Amgen, Inc |
| (r-metHuG-CSF) | | |
| floxuridine (intraarterial) | FUDR | Roche |
| (2′-deoxy-5-fluorouridine) | | |
| Fludarabine | Fludara | Berlex |
| (fluorinated nucleotide analog of the | | Laboratories, Inc., |
| antiviral agent vidarabine, 9-b-D- | | Cedar Knolls, NJ |
| arabinofuranosyladenine (ara-A)) | | |
| Fluorouracil, 5-FU | Adrucil | ICN |
| (5-fluoro-2,4(1H,3H)-pyrimidinedione) | | Pharmaceuticals, |
| | | Inc., Humacao, |
| | | Puerto Rico |
| Fulvestrant | Faslodex | IPR |
| (7-alpha-[9-(4,4,5,5,5-penta | | Pharmaceuticals, |
| fluoropentylsulphinyl) nonyl]estra-1,3,5- | | Guayama, Puerto |
| (10)-triene-3,17-beta-diol) | | Rico |
| Gemcitabine | Gemzar | Eli Lilly |
| (2′-deoxy-2′,2′-difluorocytidine | | |
| monohydrochloride (b-isomer)) | | |
| Gemtuzumab Ozogamicin | Mylotarg | Wyeth Ayerst |
| (anti-CD33 hP67.6) | | |
| Goserelin acetate | Zoladex | AstraZeneca |
| (acetate salt of [D- | Implant | Pharmaceuticals |
| Ser(But)⁶,Azgly¹⁰]LHRH; pyro-Glu-His- | | |
| Trp-Ser-Tyr-D-Ser(But)-Leu-Arg-Pro- | | |
| Azgly-NH2 acetate [C₅₉H₈₄N₁₈O₁₄•(C₂H₄O₂)ₓ | | |
| Hydroxyurea | Hydrea | Bristol-Myers |
| | | Squibb |
| Ibritumomab Tiuxetan | Zevalin | Biogen IDEC, |
| (immunoconjugate resulting from a | | Inc., Cambridge |
| thiourea covalent bond between the | | MA |
| monoclonal antibody Ibritumomab and the | | |
| linker-chelator tiuxetan [N-[2- | | |
| bis(carboxymethyl)amino]-3-(p- | | |
| isothiocyanatophenyl)-propyl]-[N-[2- | | |
| bis(carboxymethyl)amino]-2-(methyl)- | | |
| ethyl]glycine) | | |
| Idarubicin | Idamycin | Pharmacia & |
| (5,12-Naphthacenedione, 9-acetyl-7-[(3- | | Upjohn Company |
| amino-2,3,6-trideoxy-(alpha)-L-lyxo- | | |
| hexopyranosyl)oxy]-7,8,9,10-tetrahydro- | | |
| 6,9,11-trihydroxyhydrochloride, (7S-cis)) | | |
| Ifosfamide | IFEX | Bristol-Myers |
| (3-(2-chloroethyl)-2-[(2- | | Squibb |
| chloroethyl)amino]tetrahydro-2H-1,3,2- | | |
| oxazaphosphorine 2-oxide) | | |
| Imatinib Mesilate | Gleevec | Novartis AG, |
| (4-[(4-Methyl-1-piperazinyl)methyl]-N- | | Basel, Switzerland |
| [4-methyl-3-[[4-(3-pyridinyl)-2- | | |
| pyrimidinyl]amino]-phenyl]benzamide | | |
| methanesulfonate) | | |
| Interferon alfa-2a | Roferon-A | Hoffmann-La |
| (recombinant peptide) | | Roche, Inc., |
| | | Nutley, NJ |
| Interferon alfa-2b | Intron A | Schering AG, |
| (recombinant peptide) | (Lyophilized | Berlin, Germany |
| | Betaseron) | |
| Irinotecan HCl | Camptosar | Pharmacia & |
| ((4S)-4,11-diethyl-4-hydroxy-9-[(4- | | Upjohn Company |
| piperi-dinopiperidino)carbonyloxy]-1H- | | |
| pyrano[3′,4′:6,7] indolizino[1,2-b] | | |
| quinoline-3,14(4H,12H) dione | | |
| hydrochloride trihydrate) | | |
| Letrozole | Femara | Novartis |
| (4,4′-(1H-1,2,4-Triazol-1-ylmethylene) | | |
| dibenzonitrile) | | |
| Leucovorin | Wellcovorin, | Immunex, Corp., |
| (L-Glutamic acid, N[4[[(2amino-5-formyl- | Leucovorin | Seattle, WA |
| 1,4,5,6,7,8 hexahydro4oxo6- | | |
| pteridinyl)methyl]amino]benzoyl], | | |
| calcium salt (1:1)) | | |
| Levamisole HCl | Ergamisol | Janssen Research |
| ((−)-(S)-2,3,5,6-tetrahydro-6- | | Foundation, |
| phenylimidazo [2,1-b] thiazole | | Titusville, NJ |
| monohydrochloride C₁₁H₁₂N₂S•HCl) | | |
| Lomustine | CeeNU | Bristol-Myers |
| (1-(2-chloro-ethyl)-3-cyclohexyl-1- | | Squibb |
| nitrosourea) | | |
| Meclorethamine, nitrogen mustard | Mustargen | Merck |
| (2-chloro-N-(2-chloroethyl)-N- | | |
| methylethanamine hydrochloride) | | |
| Megestrol acetate | Megace | Bristol-Myers |
| 17α(acetyloxy)-6-methylpregna-4,6- | | Squibb |
| diene-3,20-dione | | |
| Melphalan, L-PAM | Alkeran | GlaxoSmithKline |
| (4-[bis(2-chloroethyl) amino]-L- | | |
| phenylalanine) | | |
| Mercaptopurine, 6-MP | Purinethol | GlaxoSmithKline |
| (1,7-dihydro-6H-purine-6-thione | | |
| monohydrate) | | |
| Mesna | Mesnex | Asta Medica |
| (sodium 2-mercaptoethane sulfonate) | | |
| Methotrexate | Methotrexate | Lederle |
| (N-[4-[[(2,4-diamino-6- | | Laboratories |
| pteridinyl)methyl]methylamino]benzoyl]- | | |
| L-glutamic acid) | | |
| Methoxsalen | Uvadex | Therakos, Inc., |
| (9-methoxy-7H-furo[3,2-g][1]- | | Way Exton, Pa |
| benzopyran-7-one) | | |
| Mitomycin C | Mutamycin | Bristol-Myers |
| | | Squibb |
| mitomycin C | Mitozytrex | SuperGen, Inc., |
| | | Dublin, CA |
| Mitotane | Lysodren | Bristol-Myers |
| (1,1-dichloro-2-(o-chlorophenyl)-2-(p- | | Squibb |
| chlorophenyl) ethane) | | |
| Mitoxantrone | Novantrone | Immunex |
| (1,4-dihydroxy-5,8-bis[[2-[(2- | | Corporation |
| hydroxyethyl)amino]ethyl]amino]-9,10- | | |
| anthracenedione dihydrochloride) | | |
| Nandrolone phenpropionate | Durabolin-50 | Organon, Inc., |
| | | West Orange, NJ |
| Nofetumomab | Verluma | Boehringer |
| | | Ingelheim Pharma |
| | | KG, Germany |
| Oprelvekin | Neumega | Genetics Institute, |
| (IL-11) | | Inc., Alexandria, |
| | | VA |
| Oxaliplatin | Eloxatin | Sanofi |
| (cis-[(1R,2R)-1,2-cyclohexanediamine- | | Synthelabo, Inc., |
| N,N′] [oxalato(2-)-O,O′] platinum) | | NY, NY |
| Paclitaxel | TAXOL | Bristol-Myers |
| (5β,20-Epoxy-1,2a,4,7β,10β,13a- | | Squibb |
| hexahydroxytax-11-en-9-one 4,10- | | |
| diacetate 2-benzoate 13-ester with (2R,3S)-N- | | |
| benzoyl-3-phenylisoserine) | | |
| Pamidronate | Aredia | Novartis |
| (phosphonic acid (3-amino-1- | | |
| hydroxypropylidene) bis-, disodium salt, | | |
| pentahydrate, (APD)) | | |
| Pegademase | Adagen | Enzon |
| ((monomethoxypolyethylene glycol | (Pegademase | Pharmaceuticals, |
| succinimidyl) 11-17-adenosine | Bovine) | Inc., Bridgewater, |
| deaminase) | | NJ |
| Pegaspargase | Oncaspar | Enzon |
| (monomethoxypolyethylene glycol | | |
| succinimidyl L-asparaginase) | | |
| Pegfilgrastim | Neulasta | Amgen, Inc |
| (covalent conjugate of recombinant | | |
| methionyl human G-CSF (Filgrastim) and | | |
| monomethoxypolyethylene glycol) | | |
| Pentostatin | Nipent | Parke-Davis |
| | | Pharmaceutical |
| | | Co., Rockville, |
| | | MD |
| Pipobroman | Vercyte | Abbott |
| | | Laboratories, |
| | | Abbott Park, IL |
| Plicamycin, Mithramycin | Mithracin | Pfizer, Inc., NY, |
| (antibiotic produced by Streptomyces | | NY |
| plicatus) | | |
| Porfimer sodium | Photofrin | QLT |
| | | Phototherapeutics, |
| | | Inc., Vancouver, |
| | | Canada |
| Procarbazine | Matulane | Sigma Tau |
| (N-isopropyl-μ-(2-methylhydrazino)-p- | | Pharmaceuticals, |
| toluamide monohydrochloride) | | Inc., Gaithersburg, |
| | | MD |
| Quinacrine | Atabrine | Abbott Labs |
| (6-chloro-9-(1-methyl-4-diethyl-amine) | | |
| butylamino-2-methoxyacridine) | | |
| Rasburicase | Elitek | Sanofi- |
| (recombinant peptide) | | Synthelabo, Inc., |
| Rituximab | Rituxan | Genentech, Inc., |
| (recombinant anti-CD20 antibody) | | South San |
| | | Francisco, CA |
| Sargramostim | Prokine | Immunex Corp |
| (recombinant peptide) | | |
| Streptozocin | Zanosar | Pharmacia & |
| (streptozocin 2-deoxy-2- | | Upjohn Company |
| [[(methylnitrosoamino)carbonyl]amino]- | | |
| a(and b)-D-glucopyranose and 220 mg | | |
| citric acid anhydrous) | | |
| Talc | Sclerosol | Bryan, Corp., |
| (Mg₃Si₄O₁₀(OH)₂) | | Woburn, MA |
| Tamoxifen | Nolvadex | AstraZeneca |
| ((Z)2-[4-(1,2-diphenyl-1-butenyl) | | Pharmaceuticals |
| phenoxy]-N,N-dimethylethanamine 2- | | |
| hydroxy-1,2,3-propanetricarboxylate | | |
| (1:1)) | | |
| Temozolomide | Temodar | Schering |
| (3,4-dihydro-3-methyl-4-oxoimidazo[5,1- | | |
| d]-as-tetrazine-8-carboxamide) | | |
| teniposide, VM-26 | Vumon | Bristol-Myers |
| (4′-demethylepipodophyllotoxin 9-[4,6-0- | | Squibb |
| (R)-2-thenylidene-(beta)-D- | | |
| glucopyranoside]) | | |
| Testolactone | Teslac | Bristol-Myers |
| (13-hydroxy-3-oxo-13,17-secoandrosta- | | Squibb |
| 1,4-dien-17-oic acid [dgr]-lactone) | | |
| Thioguanine, 6-TG | Thioguanine | GlaxoSmithKline |
| (2-amino-1,7-dihydro-6H-purine-6- | | |
| thione) | | |
| Thiotepa | Thioplex | Immunex |
| (Aziridine, 1,1′,1″- | | Corporation |
| phosphinothioylidynetris-, or Tris (1- | | |
| aziridinyl) phosphine sulfide) | | |
| Topotecan HCl | Hycamtin | GlaxoSmithKline |
| ((S)-10-[(dimethylamino) methyl]-4-ethyl- | | |
| 4,9-dihydroxy-1H-pyrano[3′,4′:6,7] | | |
| indolizino [1,2-b] quinoline-3,14- | | |
| (4H,12H)-dione monohydrochloride) | | |
| Toremifene | Fareston | Roberts |
| (2-(p-[(Z)-4-chloro-1,2-diphenyl-1- | | Pharmaceutical |
| butenyl]-phenoxy)-N,N- | | Corp., Eatontown, |
| dimethylethylamine citrate (1:1)) | | NJ |
| Tositumomab, I 131 Tositumomab | Bexxar | Corixa Corp., |
| (recombinant murine immunotherapeutic | | Seattle, WA |
| monoclonal IgG₂ₐ lambda anti-CD20 | | |
| antibody (I 131 is a | | |
| radioimmunotherapeutic antibody)) | | |
| Trastuzumab | Herceptin | Genentech, Inc |
| (recombinant monoclonal IgG₁ kappa anti- | | |
| HER2 antibody) | | |
| Tretinoin, ATRA | Vesanoid | Roche |
| (all-trans retinoic acid) | | |
| Uracil Mustard | Uracil | Roberts Labs |
| | Mustard | |
| | Capsules | |
| Valrubicin, N-trifluoroacetyladriamycin- | Valstar | Anthra --> |
| 14-valerate | | Medeva |
| ((2S-cis)-2-[1,2,3,4,6,11-hexahydro- | | |
| 2,5,12-trihydroxy-7 methoxy-6,11-dioxo- | | |
| [[4 2,3,6-trideoxy-3-[(trifluoroacetyl)- | | |
| amino-α-L-lyxo-hexopyranosyl]oxyl]-2- | | |
| naphthacenyl]-2-oxoethyl pentanoate) | | |
| Vinblastine, Leurocristine | Velban | Eli Lilly |
| (C₄₆H₅₆N₄O₁₀•H₂SO₄) | | |
| Vincristine | Oncovin | Eli Lilly |
| (C₄₆H₅₆N₄O₁₀•H₂SO₄) | | |
| Vinorelbine | Navelbine | GlaxoSmithKline |
| (3′,4′-didehydro-4′-deoxy-C′- | | |
| norvincaleukoblastine [R-(R*,R*)-2,3- | | |
| dihydroxybutanedioate (1:2)(salt)]) | | |
| Zoledronate, Zoledronic acid | Zometa | Novartis |
| ((1-Hydroxy-2-imidazol-1-yl- | | |
| phosphonoethyl) phosphonic acid | | |
| monohydrate) | | |

Chemotherapeutic agents further include compounds which have been identified to have anticancer activity but are not currently approved by the U.S. Food and Drug Administration or other counterpart agencies or are undergoing evaluation for new uses. Examples include, but are not limited to, 3-AP, 12-O-tetradecanoylphorbol-13-acetate, 17AAG, 852A, ABI-007, ABR-217620, ABT-751, ABT-263, ABT-737, ADI-PEG 20, AE-941, AG-013736, AGRO100, alanosine, AMG 706, antibody G250, antineoplastons, AP23573, apaziquone, APC8015, atiprimod, ATN-161, atrasenten, azacitidine, BB-10901, BCX-1777, bevacizumab, BG00001, bicalutamide, BMS 247550, bortezomib, bryostatin-1, buserelin, calcitriol, CCI-779, CDB-2914, cefixime, cetuximab, CG0070, cilengitide, clofarabine, combretastatin A4 phosphate, CP-675,206, CP-724,714, CpG 7909, curcumin, decitabine, DENSPM, doxercalciferol, E7070, E7389, ecteinascidin 743, efaproxiral, eflornithine, EKB-569, enzastaurin, erlotinib, exisulind, fenretinide, flavopiridol, fludarabine, flutamide, fotemustine, FR901228, G17DT, galiximab, gefitinib, genistein, glufosfamide, GTI-2040, histrelin, HKI-272, homoharringtonine, HSPPC-96, hu14.18-interleukin-2 fusion protein, HuMax-CD4, iloprost, imiquimod, infliximab, interleukin-12, IPI-504, irofulven, ixabepilone, lapatinib, lestaurtinib, leuprolide, LMB-9 immunotoxin, lonafarnib, luniliximab, mafosfamide, MB07133, MDX-010, MLN2704, monoclonal antibody 3F8, monoclonal antibody J591, motexafin, MS-275, MVA-MUC1-IL2, nilutamide, nitrocamptothecin, nolatrexed dihydrochloride, nolvadex, NS-9,06-benzylguanine, oblimersen sodium, ONYX-015, oregovomab, OSI-774, panitumumab, paraplatin, PD-0325901, pemetrexed, PHY906, pioglitazone, pirfenidone, pixantrone, PS-341, PSC 833, PXD101, pyrazoloacridine, R115777, RAD001, ranpirnase, rebeccamycin analogue, rhuAngiostatin protein, rhuMab 2C4, rosiglitazone, rubitecan, S-1, S-8184, satraplatin, SB-, 15992, SGN-0010, SGN-40, sorafenib, SR31747A, ST1571, SU011248, suberoylanilide hydroxamic acid, suramin, talabostat, talampanel, tariquidar, temsirolimus, TGFa-PE38 immunotoxin, thalidomide, thymalfasin, tipifarnib, tirapazamine, TLK286, trabectedin, trimetrexate glucuronate, TroVax, UCN-1, valproic acid, vinflunine, VNP40101M, volociximab, vorinostat, VX-680, ZD1839, ZD6474, zileuton, and zosuquidar trihydrochloride.

For a more detailed description of anticancer agents and other therapeutic agents, those skilled in the art are referred to any number of instructive manuals including, but not limited to, the Physician's Desk Reference and to Goodman and Gilman's “Pharmaceutical Basis of Therapeutics” ninth edition, Eds. Hardman et al., 1996.

The term “chemotherapy” as used herein refers to administration of a chemotherapeutic agent to a patient in need thereof.

The term “radiotherapeutic agent,” as used herein refers any type of radiation therapy known to the clinical practitioner of ordinary skill in the art used for the treatment or amelioration of cancer and/or as an inducer of apoptosis in a patient. The invention is not limited by the types, amounts, or delivery and administration systems used to deliver the therapeutic dose of radiation to the patient. For example, the patient may receive photon radiotherapy, particle beam radiation therapy, radioisotope therapy (e.g., radioconjugates with monoclonal antibodies), other types of radiotherapies, and combinations thereof. In some embodiments, the radiation is delivered to the patient using a linear accelerator. In still other embodiments, the radiation is delivered using a gamma knife.

The source of radiation can be external or internal to the patient. External radiation therapy is most common and involves directing a beam of high-energy radiation to a tumor site through the skin using, for instance, a linear accelerator. While the beam of radiation is localized to the tumor site, it is nearly impossible to avoid exposure of normal, healthy tissue. However, external radiation is usually well tolerated by patients. Internal radiation therapy involves implanting a radiation-emitting source, such as beads, wires, pellets, capsules, particles, and the like, inside the body at or near the tumor site including the use of delivery systems that specifically target cancer cells (e.g., using particles attached to cancer cell binding ligands). Such implants can be removed following treatment, or left in the body inactive. Types of internal radiation therapy include, but are not limited to, brachytherapy, interstitial irradiation, intracavity irradiation, radioimmunotherapy, and the like.

Methods of administering and apparatuses and compositions useful for external-beam radiation therapy can be found in U.S. Pat. Nos. 6,449,336, 6,398,710, 6,393,096, 6,335,961, 6,307,914, 6,256,591, 6,245,005, 6,038,283, 6,001,054, 5,802,136, 5,596,619, and 5,528,652.

The patient may optionally receive radiosensitizers (e.g., metronidazole, misonidazole, intra-arterial Budr, intravenous iododeoxyuridine (IudR), nitroimidazole, 5-substituted-4-nitroimidazoles, 2H-isoindolediones, [[(2-bromoethyl)-amino]methyl]-nitro-1H-imidazole-1-ethanol, nitroaniline derivatives, DNA-affinic hypoxia selective cytotoxins, halogenated DNA ligand, 1,2,4 benzotriazine oxides, 2-nitroimidazole derivatives, fluorine-containing nitroazole derivatives, benzamide, nicotinamide, acridine-intercalator, 5-thiotretrazole derivative, 3-nitro-1,2,4-triazole, 4,5-dinitroimidazole derivative, hydroxylated texaphrins, cisplatin, mitomycin, tiripazamine, nitrosourea, mercaptopurine, methotrexate, fluorouracil, bleomycin, vincristine, carboplatin, epirubicin, doxorubicin, cyclophosphamide, vindesine, etoposide, paclitaxel, heat (hyperthermia), and the like), radioprotectors (e.g., cysteamine, aminoalkyl dihydrogen phosphorothioates, amifostine (WR 2721), IL-1, IL-6, and the like). Radiosensitizers enhance the killing of tumor cells. Radioprotectors protect healthy tissue from the harmful effects of radiation.

Any type of radiation can be administered to a patient, so long as the dose of radiation is tolerated by the patient without unacceptable negative side-effects. Suitable types of radiotherapy include, for example, ionizing (electromagnetic) radiotherapy (e.g., X-rays or gamma rays) or particle beam radiation therapy (e.g., high linear energy radiation). Ionizing radiation is defined as radiation comprising particles or photons that have sufficient energy to produce ionization, i.e., gain or loss of electrons (as described in, for example, U.S. Pat. No. 5,770,581 incorporated herein by reference in its entirety). The effects of radiation can be at least partially controlled by the clinician. The dose of radiation is preferably fractionated for maximal target cell exposure and reduced toxicity.

The total dose of radiation administered to the patient typically is about 0.01 Gray (Gy) to about 100 Gy. More typically, about 10 Gy to about 65 Gy (e.g., about 15 Gy, 20 Gy, 25 Gy, 30 Gy, 35 Gy, 40 Gy, 45 Gy, 50 Gy, 55 Gy, or 60 Gy) are administered over the course of treatment. While in some embodiments a complete dose of radiation can be administered over the course of one day, the total dose is ideally fractionated and administered over several days. Desirably, radiotherapy is administered over the course of at least about 3 days, e.g., at least 5, 7, 10, 14, 17, 21, 25, 28, 32, 35, 38, 42, 46, 52, or 56 days (about 1-8 weeks). Accordingly, a daily dose of radiation will comprise approximately 1-5 Gy (e.g., about 1 Gy, 1.5 Gy, 1.8 Gy, 2 Gy, 2.5 Gy, 2.8 Gy, 3 Gy, 3.2 Gy, 3.5 Gy, 3.8 Gy, 4 Gy, 4.2 Gy, or 4.5 Gy), preferably 1-2 Gy (e.g., 1.5-2 Gy). The daily dose of radiation should be sufficient to induce destruction of the targeted cells. If stretched over a period, radiation preferably is not administered every day, thereby allowing the animal to rest and the effects of the therapy to be realized. For example, radiation desirably is administered on 5 consecutive days, and not administered on 2 days, for each week of treatment, thereby allowing 2 days of rest per week. However, radiation can be administered 1 day/week, 2 days/week, 3 days/week, 4 days/week, 5 days/week, 6 days/week, or all 7 days/week, depending on the animal's responsiveness and any potential side effects. Radiation therapy can be initiated at any time in the therapeutic period. Preferably, radiation is initiated in week 1 or week 2, and is administered for the remaining duration of the therapeutic period. For example, radiation is administered in weeks 1-6 or in weeks 2-6 of a therapeutic period comprising 6 weeks for treating, for instance, a solid tumor. Alternatively, radiation is administered in weeks 1-5 or weeks 2-5 of a therapeutic period comprising 5 weeks. These exemplary radiotherapy administration schedules are not intended, however, to limit the present invention.

In certain embodiments of the invention involving radiotherapy, the present invention provides methods comprising the co-administration of gossypol and a therapeutically effective dose of external-beam radiation therapy. The external-beam radiation therapy can be generated or manipulated by any means known to one of skill in the art.

It will be appreciated that both the particular radiation dose to be utilized in treating cancer and the method of administration will depend on a variety of factors. Thus, the dosages of radiation that can be used according to the methods of the present invention are determined by the particular requirements of each situation. The dosage will depend on such factors as the size of the tumor, the location of the tumor, the age and sex of the patient, the frequency of the dosage, the presence of other tumors, possible metastases and the like. Those skilled in the art of radiotherapy can readily ascertain the dosage and the method of administration for any particular tumor by reference to Hall, E. J., Radiobiology for the Radiobiologist, 5th edition, Lippincott Williams & Wilkins Publishers, Philadelphia, Pa., 2000; Gunderson, L. L. and Tepper J. E., eds., Clinical Radiation Oncology, Churchill Livingstone, London, England, 2000; and Grosch, D. S., Biological Effects of Radiation, 2nd edition, Academic Press, San Francisco, Calif., 1980.

The term “radiotherapy,” as used herein refers to administration of a radiotherapeutic agent to a patient in need thereof.

Patients which may be treated with gossypol or gossypol in combination with one or more additional therapeutic agents according to the methods of present invention include mammals, e.g., humans, although the invention is not intended to be so limited. Other patients include veterinary animals (cows, sheep, pigs, horses, dogs, cats and the like).

The term “therapeutically effective amount,” as used herein refers to the amount of a given therapeutic agent sufficient to result in amelioration of one or more symptoms of a disorder or condition, or prevent appearance or advancement of a disorder or condition, or cause regression of or cure from the disorder or condition.

In certain embodiments of the invention, a therapeutically effective amount refers to the amount of gossypol administered to a patient having cancer that decreases the rate of tumor growth, decreases tumor mass, decreases the number of metastases, increases time to tumor progression, or increases survival time of said patient by at least about 5% or more, e.g., at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, or more.

In additional embodiments, a therapeutically effective amount refers to the amount of gossypol in combination with one or more additional therapeutic agents administered to a patient having cancer that decreases the rate of tumor growth, decreases tumor mass, decreases the number of metastases, increases time to tumor progression, or increases survival time of said patient by at least about 5% or more, e.g., at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, or more.

The terms “sensitize” and “sensitizing,” as used herein, refer to making, through the administration of gossypol, a patient or a cell within a patient more susceptible, or more responsive, to the biological effects (e.g., promotion or retardation of an aspect of cellular function including, but not limited to, cell growth, proliferation, invasion, angiogenesis, or apoptosis) of one or more additional therapeutic agents. The sensitizing effect of gossypol on a target cell can be measured as the difference in the intended biological effect (e.g., promotion or retardation of an aspect of cellular function including, but not limited to, cell growth, proliferation, invasion, angiogenesis, or apoptosis) observed upon the administration of a second therapeutic agent with and without administration of gossypol. The response of the sensitized cell can be increased by at least about 5% or more, e.g., at least about 10%, at least about 20%, at least about 30%, at least about 40%, at least about 50%, at least about 60%, at least about 70%, at least about 80%, at least about 90%, at least about 100%, at least about 150%, at least about 200%, at least about 350%, at least about 300%, at least about 350%, at least about 400%, at least about 450%, or at least about 500%, or more, over the response in the absence of gossypol. In one aspect of the invention, the administration of gossypol sensitizes a patient more susceptible or more responsive to the biological effects of ABT-737 and related agents.

The term “dysregulation of apoptosis,” as used herein, refers to any aberration in the ability of (e.g., predisposition) a cell to undergo cell death via apoptosis. Apoptosis has many roles in development and homeostasis and its dysregulation is a hallmark of many diseases (Peter et al., Proc. Natl. Acad. Sci. USA 94:12736 (1997)). Dysregulation of apoptosis is associated with or induced by a variety of conditions, including for example, autoimmune disorders (e.g., systemic lupus erythematosus, rheumatoid arthritis, graft-versus-host disease, myasthenia gravis, or Sjögren's syndrome), chronic inflammatory conditions (e.g., psoriasis, asthma or Crohn's disease), hyperproliferative disorders (e.g., tumors, B cell lymphomas, or T cell lymphomas), viral infections (e.g., herpes, papilloma, or HIV), microbial infections, parasitic infections and other conditions such as osteoarthritis and atherosclerosis. It should be noted that when the dysregulation is induced by or associated with a viral infection, the viral infection may or may not be detectable at the time dysregulation occurs or is observed. That is, viral-induced dysregulation can occur even after the disappearance of symptoms of viral infection.

The term “hyperproliferative disease,” as used herein refers to any condition in which a localized population of proliferating cells in an animal is not governed by the usual limitations of normal growth, such as but not limited to cancer. Non-limiting examples of hyperproliferative diseases include psoriasis, restenosis, cancers, tumors, neoplasms, lymphomas, and the like. A neoplasm is said to be benign if it does not undergo invasion or metastasis and malignant if it does either of these. A “metastatic” cell means that the cell can invade and destroy neighboring body structures. Hyperplasia is a form of cell proliferation involving an increase in cell number in a tissue or organ without significant alteration in structure or function. Metaplasia is a form of controlled cell growth in which one type of fully differentiated cell substitutes for another type of differentiated cell.

The term “cancer,” as used herein, is intended to refer to any known cancer, and may include, but is not limited to the following: leukemias such as acute leukemia, acute lymphocytic leukemia, acute myelocytic leukemias such as myeloblastic, promyelocytic, myelomonocytic, monocytic, and erythroleukemia leukemias, and myelodysplastic syndrome; chronic leukemias such as chronic myelocytic (granulocytic) leukemia, chronic lymphocytic leukemia, and hairy cell leukemia; polycythemia vera; lymphomas such as Hodgkin's disease and non-Hodgkin's disease; multiple myelomas such as smoldering multiple myeloma, non-secretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone and connective tissue sarcomas such as bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, and synovial sarcoma; brain tumors such as glioma, astrocytoma, brain stem glioma, ependymoma, oligodendroglioma, nonglial tumor, acoustic neurinoma, craniopharyngioma, medulloblastoma, meningioma, pineocytoma, pineoblastoma, and primary brain lymphoma; breast cancers such as adenocarcinoma, lobular (small cell) carcinoma, intraductal carcinoma, medullary breast cancer, mucinous breast cancer, tubular breast cancer, papillary breast cancer, Paget's disease of the breast, and inflammatory breast cancer; adrenal cancers such as pheochromocytoma and adrenocortical carcinoma; thyroid cancers such as papillary or follicular thyroid cancer, medullary thyroid cancer and anaplastic thyroid cancer; pancreatic cancers such as insulinoma, gastrinoma, glucagonoma, vipoma, somatostatin-secreting tumor, and carcinoid or islet cell tumor; pituitary cancers such as prolactin-secreting tumor and acromegaly; eye cancers such as ocular melanoma, iris melanoma, choroidal melanoma, and cilliary body melanoma, and retinoblastoma; vaginal cancers such as squamous cell carcinoma, adenocarcinoma, and melanoma; vulvar cancers such as squamous cell carcinoma, melanoma, adenocarcinoma, basal cell carcinoma, sarcoma, and Paget's disease of the genitals; cervical cancers such as squamous cell carcinoma and adenocarcinoma; uterine cancers such as endometrial carcinoma and uterine sarcoma; ovarian cancers such as ovarian epithelial carcinoma, ovarian epithelial borderline tumor, germ cell tumor, and stromal tumor; esophageal cancers such as squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers such as adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers such as hepatocellular carcinoma and hepatoblastoma, gallbladder cancers such as adenocarcinoma; cholangiocarcinomas such as papillary, nodular, and diffuse; lung cancers such as non-small cell lung cancer, squamous cell carcinoma (epidermoid carcinoma), adenocarcinoma, large-cell carcinoma and small-cell lung cancer; testicular cancers such as germinal tumor, seminoma, anaplastic, classic (typical), spermatocytic, nonseminoma, embryonal carcinoma, teratoma carcinoma, and choriocarcinoma (yolk-sac tumor), prostate cancers such as adenocarcinoma, leiomyosarcoma, and rhabdomyosarcoma; penile cancers; oral cancers such as squamous cell carcinoma; basal cancers; salivary gland cancers such as adenocarcinoma, mucoepidermoid carcinoma, and adenoidcystic carcinoma; pharynx cancers such as squamous cell cancer and verrucous; skin cancers such as basal cell carcinoma, squamous cell carcinoma and melanoma, superficial spreading melanoma, nodular melanoma, lentigo malignant melanoma, acral lentiginous melanoma; head and neck cancers; kidney cancers such as renal cell cancer, adenocarcinoma, hypernephroma, fibrosarcoma, transitional cell cancer (renal pelvis and/or ureter); Wilms' tumor; and bladder cancers such as transitional cell carcinoma, squamous cell cancer, adenocarcinoma, and carcinosarcoma. In addition, cancers that can be treated by the methods and compositions of the present invention include myxosarcoma, osteogenic sarcoma, endothelio sarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinoma. See Fishman et al., 1985, Medicine, 2d Ed., J. B. Lippincott Co., Philadelphia, Pa. and Murphy et al., 1997, Informed Decisions: The Complete Book of Cancer Diagnosis, Treatment, and Recovery, Viking Penguin, New York, N.Y., for a review of such disorders.

The pathological growth of activated lymphoid cells often results in an autoimmune disorder or a chronic inflammatory condition. The term “autoimmune disorder” as used herein refers to any condition in which an organism produces antibodies or immune cells which recognize the organism's own molecules, cells or tissues. Non-limiting examples of autoimmune disorders include autoimmune hemolytic anemia, autoimmune hepatitis, Berger's disease or IgA nephropathy, celiac sprue, chronic fatigue syndrome, Crohn's disease, dermatomyositis, fibromyalgia, graft versus host disease, Grave's disease, Hashimoto's thyroiditis, idiopathic thrombocytopenia purpura, lichen planus, multiple sclerosis, myasthenia gravis, psoriasis, rheumatic fever, rheumatic arthritis, scleroderma, Sjögren's syndrome, systemic lupus erythematosus, type 1 diabetes, ulcerative colitis, vitiligo, and the like.

The term “neoplastic disease,” as used herein, refers to any abnormal growth of cells being either benign (non-cancerous) or malignant (cancerous).

The term “anti-neoplastic agent,” as used herein, refers to any compound that retards the proliferation, growth, or spread of a targeted (e.g., malignant) neoplasm.

The terms “prevent,” “preventing” and “prevention,” as used herein, refer to a decrease in the occurrence of pathological cells (e.g., hyperproliferative or neoplastic cells) in an animal, e.g., a human. The prevention may be complete, e.g., the total absence of pathological cells in a subject. The prevention may also be partial, such that the occurrence of pathological cells in a subject is less than that which would have occurred without the present invention.

The term “synergistic,” as used herein, refers to a therapeutic effect obtained when gossypol and one or more additional therapeutic agents are administered together (e.g., at the same time or one after the other) that is greater than the additive effect of gossypol and the one or more additional therapeutic agents when administered individually. The synergistic effect allows for lower doses of gossypol and/or the additional therapeutic agent(s) to be administered and/or provides greater efficacy at the same doses. The synergistic effect obtained can be at least about 5%, at least about 10%, at least about 20%, at least about 30%, at least about 40%, at least about 50%, at least about 60%, at least about 70%, at least about 80%, at least about 90%, at least about 100%, at least about 125%, at least about 150%, at least about 175%, at least about 200%, at least about 250%, at least about 300%, at least about 350%, at least about 400%, or at least about 500% more than the additive effect of gossypol and the additional therapeutic agent(s) when administered individually. For example, with respect to the treatment of cancer, the synergistic effect can be a decrease in the rate of tumor growth, a decrease in tumor mass, a decrease in the number of metastases, an increase in time to tumor progression, or an increase in survival time. In certain embodiments of the invention, the co-administration of gossypol by pulsatile dosing and one or more anticancer agents (e.g., one or more additional chemotherapeutic agents and/or radiotherapeutic agents) may allow for the use of lower doses of gossypol and/or the anticancer agents such that the cancer is effectively treated while avoiding any substantial toxicity to the subject.

The term “about,” as used herein, includes the recited number +/−10%. Thus, “about 0.5” means 0.45 to 0.55.

The terms “co-administration,” or “co-administering” as used herein refer to the simultaneous administration of gossypol in combination with one or more additional therapeutic agents, either as a single pharmaceutical composition or separate pharmaceutical compositions. As separate pharmaceutical compositions, gossypol and one or more additional therapeutic agents can be co-administered to the patient under one or more of the following conditions: at different periodicities, at different durations, at different concentrations, by different administration routes, etc. Thus, the terms “co-administration,” or “co-administering,” also refer to administration of gossypol prior to or after the administration of the one or more additional therapeutic agents, e.g., 1, 2, 3, 4, 5, 6, 12, or 18 hours, 1, 2, 3, 4, 5, or 6 days, or 1, 2, 3, or 4 weeks before or after the second therapeutic agent(s). In one embodiment of the invention, gossypol and the one or more therapeutic agents are administered concurrently but on different schedules, e.g., gossypol is administered on at least three consecutive days followed by at least seventeen days wherein gossypol is not administered while the one or more additional therapeutic agents are administered daily, once a week, once every two weeks, once every three weeks, or once every four weeks, etc. In a particular embodiment, (−)-gossypol is orally administered twice-a-day for three consecutive days followed by eighteen consecutive days wherein (−)-gossypol is not administered, and a taxane (e.g., docetaxel or paclitaxel) is intravenously administered every twenty one days. In another embodiment, gossypol is co-administered with ABT-737 or a related compound.

The term “pulsatile dose administration,” as used herein refers to intermittent (i.e., not constant daily) administration of gossypol. Pulsatile dose administration schedules encompass any discontinuous daily administration regimen that provides a therapeutically effective amount of gossypol to a patient in need thereof. Pulsatile dosing regimens may use equivalent, lower or higher doses of gossypol than typically used in continuous daily dosing regimens. On the days that gossypol is scheduled to be administered, administration of gossypol may occur once a day, twice-a-day (i.e., BID), three times a day, four times a day or more in accordance with an intermittent daily dosing schedule.

In one example of pulsatile dose administration, gossypol is administered on a least two consecutive days, e.g., at least two, three, four, five, six or seven consecutive days, followed by at least one day, at least two consecutive days, at least three consecutive days, at least four consecutive days, at least five consecutive days, at least six consecutive days, at least seven consecutive days, at least eight consecutive days, at least nine consecutive days, at least ten consecutive days, at least eleven consecutive days, at least twelve consecutive days, at least thirteen consecutive days, at least fourteen consecutive days, at least fifteen consecutive days, at least sixteen consecutive days, at least seventeen consecutive days, at least eighteen consecutive days, at least nineteen consecutive days, at least twenty consecutive days, at least three consecutive weeks, at least four consecutive weeks, or longer wherein gossypol is not administered. The pulsatile dose administration of gossypol according to such a schedule can continue for one, two, three or four weeks, one, two, three or four months, one, two, three or four years or longer.

In another example, gossypol is administered twice-a-day for three consecutive days followed by at least one day, at least two consecutive days, at least three consecutive days, at least four consecutive days, at least five consecutive days, at least six consecutive days, at least seven consecutive days, at least eight consecutive days, at least nine consecutive days, at least ten consecutive days, at least eleven consecutive days, at least twelve consecutive days, at least thirteen consecutive days, at least fourteen consecutive days, at least fifteen consecutive days, at least sixteen consecutive days, at least seventeen consecutive days, at least eighteen consecutive days, at least nineteen consecutive days, at least twenty consecutive days, at least three consecutive weeks, at least four consecutive weeks, or longer wherein gossypol is not administered. The administration of gossypol can continue for two, three or four weeks, one, two, three or four months, one, two, three or four years or longer.

In still another example, gossypol is administered twice-a-day for three consecutive days followed at least seventeen consecutive days, at least eighteen consecutive days, at least nineteen consecutive days, at least twenty consecutive days, or at least three consecutive weeks wherein gossypol is not administered.

The term “Bcl-2 family proteins,” as used herein, refers to both the anti-apoptotic members of the Bcl-2 family, including, but not limited to, Bcl-2, Bcl-xL, Mcl-1, A1/BFL-1, BOO-DIVA, Bcl-w, Bcl-6, Bcl-8, and Bcl-y, and the pro-apoptotic members of the Bcl-2 family, including, but not limited to, Bak, Bax, Bad, tBid, Hrk, Bim, Bmf, Noxa, and Puma, as well as other Bcl-2 homology domain 3 (BH3) containing proteins. As used herein, any of the members of the Bcl-2 family proteins may be referred to as “Bcl-2-like proteins.”

The term “overexpression of anti-apoptotic Bcl-2 family proteins,” as used herein, refers to an elevated level (e.g., aberrant level) of mRNAs encoding for an anti-apoptotic Bcl-2 family protein(s), and/or to elevated levels of anti-apoptotic Bcl-2 family protein(s) in cells as compared to similar corresponding non-pathological cells expressing basal levels of mRNAs encoding anti-apoptotic Bcl-2 family proteins or having basal levels of anti-apoptotic Bcl-2 family proteins. Methods for detecting the levels of mRNAs encoding anti-apoptotic Bcl-2 family proteins or levels of anti-apoptotic Bcl-2 family proteins in a cell include, but are not limited to, Western blotting using anti-apoptotic Bcl-2 family protein antibodies, immunohistochemical methods, and methods of nucleic acid amplification or direct RNA detection. As important as the absolute level of anti-apoptotic Bcl-2 family proteins in cells is to determining that they overexpress anti-apoptotic Bcl-2 family proteins, so also is the relative level of anti-apoptotic Bcl-2 family proteins to other pro-apoptotic signaling molecules (e.g., pro-apoptotic Bcl-2 family proteins) within such cells. When the balance of these two are such that, were it not for the levels of the anti-apoptotic Bcl-2 family proteins, the pro-apoptotic signaling molecules would be sufficient to cause the cells to execute the apoptosis program and die, said cells would be dependent on the anti-apoptotic Bcl-2 family proteins for their survival. In such cells, exposure to an inhibiting effective amount of an anti-apoptotic Bcl-2 family protein inhibitor will be sufficient to cause the cells to execute the apoptosis program and die. Thus, the term “overexpression of an anti-apoptotic Bcl-2 family protein” also refers to cells that, due to the relative levels of pro-apoptotic signals and anti-apoptotic signals, undergo apoptosis in response to inhibiting effective amounts of compounds that inhibit the function of anti-apoptotic Bcl-2 family proteins.

The term “c-Myc” as used herein refers to either the c-Myc gene (official symbol: MYC) or protein. c-Myc is a transcription factor of the basic-helix-loop-helix-leucine zipper (bHLH-Zip) family that regulates inter alia cell proliferation and apoptosis.

The term “Mcl-1” as used herein refers to myeloid cell leukemia sequence 1 (BCL2-related) gene (official symbol: MCL1) or protein. The protein encoded by the Mcl-1 gene belongs to the Bcl-2 family. Two transcript variants encoding distinct isoforms have been identified. The longer gene product (isoform 1) enhances cell survival by inhibiting apoptosis while the alternatively spliced shorter gene product (isoform 2) promotes apoptosis and is death-inducing.

The term “Noxa” as used herein refers to the human phorbol-12-myristate-13-acetate-induced protein 1 gene (official symbol: PMAIP1) or protein.

The term “official symbol” as used herein refers to EntrezGene database maintained by the United States National Center for Biotechnology Information.

The term “pharmaceutically acceptable carrier” encompasses any of the standard pharmaceutical carriers, buffers and excipients, including phosphate-buffered saline solution, water, and emulsions (such as an oil/water or water/oil emulsion), and various types of wetting agents and/or adjuvants. Suitable pharmaceutical carriers and their formulations are described in Remington's Pharmaceutical Sciences, Mack Publishing Co., Easton, Pa., 19th ed. 1995. Preferred pharmaceutical carriers depend upon the intended mode of administration of the active agent. Typical modes of administration are described below.

### Overview

Although (−)-gossypol has been shown to bind to Bcl-2, Bcl-xL and Mcl-1 proteins (Wang et al., Semin Oncol 30:133 (2003); Wang et al., J Med Chem 49:6139 (2006), the precise cellular mechanism of action for its anti-tumor activity of remains unclear. Thus, the cellular mechanism of action for apoptosis induction by (−)-gossypol using a panel of human breast and prostate cancer cell lines was investigated.

(−)-Gossypol induces apoptosis in both Bax/Bak-dependent and independent manners. It also demonstrated that (−)-gossypol upregulates two pro-apoptotic Bcl-2 proteins, Noxa and Puma, in all breast and prostate cancer cell lines tested. siRNA knock-down established that Noxa and Puma play an important role in apoptosis induction by (−)-gossypol in tumor cells, regardless the apoptosis induction is Bax/Bak dependent or not. Upregulation of Noxa is only observed in tumor cells, not in normal cells. This specific upregulation will provide a therapeutic window for the use of (−)-gossypol to kill tumor cells but not normal cells.

(−)-Gossypol binds to Bcl-2, Bcl-xL, and Mcl-1 proteins with low to sub-micromolar affinities (Wang et al., J. Med. Chem. 49:6139 (2006)), and can neutralize the anti-apoptotic functions of Bcl-2, Bcl-xL, and Mcl-1 through its direct binding to these proteins. It has been demonstrated that (−)-gossypol also induces upregulation of Noxa and Puma in tumor cells. Immunoprecipitation experiments showed that Noxa, but not Puma, forms a complex with Mcl-1 in 2LMP cells treated with (−)-gossypol, and there was excess amount of Noxa in the supernatant not bound to Mcl-1. Since Noxa binds to Mcl-1 with a higher affinity than (−)-gossypol, the presence of excess amount of Noxa suggested that Mcl-1 is predominantly antagonized by (−)-gossypol-induced Noxa instead of by (−)-gossypol through direct binding. Also since Puma is strongly upregulated by (−)-gossypol, and Puma has a much stronger affinity to Bcl-2 and Bcl-xL proteins than (−)-gossypol, these data raise the possibility that (−)-gossypol antagonizes Bcl-2 and Bcl-xL proteins, at least in part, through upregulation of Puma. Indeed, it was found that Puma co-complexes with Bcl-2 protein in 2LMP cells treated with (−)-gossypol.

The anti-apoptotic Bcl-2 proteins, in particular Bcl-2 and Bcl-xL, have been considered as promising cancer therapeutic targets. Recent studies have demonstrated, however, that targeting Bcl-2 and Bcl-xL proteins alone is insufficient to induce apoptosis in most tumor cells (van Delft et al., Cancer Cell 10:389 (2006); Lin et al., Oncogene 26:3972 (2007); Konopleva et al., Cancer Cell 10:375 (2006)). For example, ABT-737, a highly potent and specific small-molecule inhibitor of Bcl-2 and Bcl-xL, is only effective in a small subset of tumor cell lines examined (Oltersdorf et al., Nature 435:677 (2005)). Mcl-1 was identified as the dominant molecule that mediates the resistance of ABT-737 in cancer cells and has emerged as an important cancer therapeutic target (van Delft et al., Cancer Cell 10:389 (2006); Lin et al., Oncogene 26:3972 (2007); Konopleva et al., Cancer Cell 10:375 (2006)). Because (−)-gossypol induces upregulation of Noxa and Puma, both of which antagonize Mcl-1 protein, it was evaluated if (−)-gossypol can overcome Mcl-1 mediated resistance of cancer cells to ABT-737. The results, presented herein, showed that (−)-gossypol was highly effective in sensitizing cancer cells to ABT-737 for apoptosis induction, at concentrations at which both compounds have minimal or no activity on their own. While not being bound by theory, it is believed that (−)-gossypol overcomes Mcl-1-mediated resistance of cancer cells to ABT-737 by neutralizing Mcl-1 through Noxa.

The universal upregulation of Noxa and Puma by (−)-gossypol in tumor cells also sheds light on the molecular mechanism for its broad antitumor activity. Tumor cells are highly resistant to apoptosis induction due to high levels of anti-apoptotic proteins such as Bcl-2, Bcl-xL and/or Mcl-1. Puma binds to Bcl-2, Bcl-xL and Mcl-1 and antagonizes their function, whereas Noxa specifically targets Mcl-1 (Chen et al., Mol Cell 17:393 (2005); Certo et al., Cancer Cell 9:351 (2006)). Through upregulation of both of Puma and Noxa, (−)-gossypol counteracts the anti-apoptotic function of Bcl-2, Bcl-xL and Mcl-1, therefore inducing cell death and apoptosis on its own.

These studies also showed that upregulation of Noxa and Puma is at the transcriptional level. The mRNA of Noxa and Puma is increased by (−)-gossypol at time points preceding the cell death. It was determined, however, that several candidate transcription factors, including E2F1, HIF-1α, c-Myc and p53 are not responsible for induction of Noxa and Puma by (−)-gossypol. Genowide screening of other transcription factors may be needed to identify the transcription factor(s) for the upregulation of Noxa and Puma induced by (−)-gossypol. Although c-Myc is not the transcription factor mediating the upregulation of Noxa and Puma, the studies described herein show that knock-down of c-Myc by siRNA attenuated apoptosis induction by (−)-gossypol. Since c-Myc plays an essential role for apoptosis induction by (−)-gossypol, tumors with overexpression of c-Myc may be particularly sensitive to (−)-gossypol treatment. Also the requirement of c-Myc for apoptosis induction by (−)-gossypol sheds light on the molecular basis for its selectivity in normal cells due to their low expression of c-Myc.

These data have several implications: (1) since Noxa is selectively upregulated by (−)-gossypol in tumor cells, Noxa expression levels will serve as a surrogate marker for the anti-tumor activity of (−)-gossypol; (2) since c-Myc is required for apoptosis induction by (−)-gossypol, elevated or overexpression of c-Myc will render tumors more sensitive to (−)-gossypol treatment; and (3) since (−)-gossypol induces upregulation of Puma and Noxa, which can antagonize Bcl-2, Bcl-xL, and Mcl-1, it can be used to sensitize tumor cells to other anticancer agents, whose activity may be limited by the overexpression of anti-apoptotic Bcl-2 proteins, such as Mcl-1

### Biomarkers of the Invention

As described above, Applicants have discovered that c-Myc plays a pivotal role in mediating apoptosis induction by (−)-gossypol. Other members of the Bcl-2 family proteins, such as Mcl-1, Noxa, and Puma, also play important roles in mediating apoptosis induction by (−)-gossypol and/or serve as surrogate markers for (−)-gossypol efficacy. Thus, in one aspect, the present invention provides a biomarker for selecting a patient for treatment with gossypol, or for treatment with gossypol and one or more additional therapeutic agents, wherein said biomarker comprises an elevated expression level of c-Myc, Mcl-1, or combination thereof, relative to the normal expression levels of said c-Myc, Mcl-1, or combination thereof.

In certain embodiments of the invention, the biomarker comprises, consists essentially, or consists of an elevated expression of c-Myc relative to the normal expression level of c-Myc. In other embodiments, the biomarker comprises an elevated expression level of Mcl-1 relative to the normal expression level of Mcl-1.

In additional embodiments of the invention, the biomarker further comprises one or more additional biomarkers. In one such embodiment, the biomarker further comprises prostate specific antigen (PSA).

In another aspect, the present invention provides a biomarker for determining the efficacy of treatment of a patient with gossypol, or treatment with gossypol and one or more additional therapeutic agents, wherein said biomarker comprises the expression level of Noxa.

In additional embodiments of the invention, an elevated expression level of Noxa relative to the normal expression level of Noxa indicates that treatment of the patient with gossypol, or treatment of the patient with gossypol and one or more additional therapeutic agents is efficacious.

### Methods of the Invention

In another aspect, the present invention provides methods for selecting or assessing the suitability of a patient with a disease, condition, or disorder responsive to the treatment with gossypol comprising determining the expression level of a biomarker selected from the group consisting of c-Myc, Mcl-1, and a combination of c-Myc and Mcl-1 in said patient. In one embodiment, the method further comprises administering gossypol to said patient if the patient has an elevated expression level of said biomarker relative to the normal expression level of said biomarker. Thus, the method provides a means for practicing personalized medicine, wherein patients that are particularly likely to respond to treatment with gossypol can be selected.

In another aspect, the present invention provides methods for treating or ameliorating a disease, condition, or disorder in a patient comprising: (a) determining the expression level of a biomarker selected from the group consisting of c-Myc, Mcl-1, and a combination c-Myc and Mcl-1 in said patient; and (b) administering gossypol to the patient if said patient has an elevated expression level of said biomarker relative to the normal expression level of said biomarker. Thus, the method provides a means for practicing personalized medicine, wherein the treatment (i.e., administration of gossypol) is tailored to the patient having a disease, condition, or disorder, based on the characteristics of the disease, condition, or disorder. In this case, the disease, condition, or disorder is characterized by an elevated expression level of a biomarker, such as c-Myc, relative to the normal expression level of that biomarker. Also the amount of gossypol administered to the patient can be personalized to provide a “minimum effective dose” based on the relative expression level of c-Myc, Mcl-1, or combination thereof. For example, a cancer patient with a high relative expression level of c-Myc may be administered a lower dose of gossypol because overexpression of c-Myc renders the cancer, e.g., a tumor, more sensitive to gossypol treatment.

In additional embodiments, methods of the invention further comprise: (a) determining the expression level of one or more additional biomarkers; and (b) administering gossypol to said patient if said patient has dysregulated expression of said one or more additional biomarkers.

In additional embodiments of the invention, the amount of gossypol, or gossypol and one or more additional therapeutic agents administered to the patient is correlated with the expression level of c-Myc such that patients with high relative expression levels of c-Myc receive lower and/or less frequent doses of gossypol, or gossypol and one or more additional therapeutic agents, then patients with low relative expression levels of c-Myc.

In additional embodiments, the methods of the invention further comprise co-administering one or more additional therapeutic agents. In further embodiments, the one or more additional therapeutic agents comprise at least one or more anticancer agents. In one such embodiment, the anticancer agent is a radiotherapeutic agent. In another such embodiment, the anticancer agent is a chemotherapeutic agent.

Certain methods of the invention are used to treat diseased cells, tissues, organs, or pathological conditions and/or disease states in a patient (e.g., a mammalian subject including, but not limited to, humans and veterinary animals) characterized as overexpressing c-Myc and other Bcl-2 family proteins. In this regard, any disease, condition, or pathology associated with or caused by a dysregulation of apoptosis is amenable to treatment or prophylaxis using the present methods. A non-limiting exemplary list of these diseases and conditions includes, but is not limited to, hyperproliferative diseases, autoimmune diseases, inflammatory diseases, infectious diseases, degenerative diseases, vascular diseases, and the like. Infections suitable for treatment with the methods of the present invention include, but are not limited to, infections caused by viruses (e.g., AIDS), bacteria, fungi, parasites, mycoplasma, prions, and the like.

Thus, in one embodiment, the patient has an autoimmune disease, such as but not limited to, Hashimoto's thyroiditis, pernicious anemia, Addison's disease, type I diabetes, rheumatoid arthritis, systemic lupus erythematosus, dermatomyositis, Sjogren syndrome, lupus erythematosus, multiple sclerosis, myasthenia gravis, reactive arthritis, graft-versus-host disease, and Grave's disease.

In still another embodiment, the has an infectious disease, such as but not limited to, HIV/AIDS.

In still another embodiment, the patient has a degenerative disease, such as but not limited to, Alzheimer's Disease, amyotrophic Lateral Sclerosis (ALS), atherosclerosis, cancer, diabetes, heart disease, inflammatory bowel disease (IBD), Norrie disease, Parkinson's Disease, prostatitis, osteoarthritis, osteoporosis, and Shy-Drager syndrome.

In still another embodiment, the patient has a vascular disease, such as but not limited to, peripheral arterial disease (PAD).

In a particular aspect, the present invention provides methods for selecting or assessing the suitability of a patient with a hyperproliferative disease responsive to the treatment with gossypol comprising: (a) determining the expression level of a biomarker selected from the group consisting of c-Myc, Mcl-1, and a combination of c-Myc and Mcl-1 in said patient; and (b) administering gossypol to said patient if the patient has an elevated expression level of said biomarker relative to the normal expression level of said biomarker.

In another particular aspect, the present invention provides methods for treating or ameliorating a hyperproliferative disease in a patient comprising: (a) determining the expression level of a biomarker selected from the group consisting of c-Myc, Mcl-1, and a combination of c-Myc and Mcl-1 in said patient; and (b) administering gossypol to said patient if the patient has an elevated expression level of said biomarker relative to the normal expression level of said biomarker.

In additional embodiments, the hyperproliferative disease is cancer, particularly prostate cancer. In one such embodiment, the cancer cells, e.g., tumor cells, overexpress c-Myc.

In additional embodiments, the biomarker comprises c-Myc.

In additional embodiments, gossypol is (−)-gossypol, particularly (−)-gossypol acetic acid co-crystals.

In additional embodiments, gossypol is co-administered with one or more chemotherapeutic agents such as docetaxel or paclitaxel.

The methods of the invention can be practiced, for example, by collecting a biological sample, particularly a tumor biopsy sample from a patient having cancer, e.g., prostate cancer, and determining the expression level of c-Myc in that sample. If the expression level of c-Myc in the biological sample is higher than the normal expression level of c-Myc, then gossypol can be administered to the patient, or gossypol and one or more additional chemotherapeutic agents can be co-administered to the patient, to treat the cancer.

In another aspect, the present invention provides methods for overcoming Mcl-1-mediated chemoresistance to chemotherapy in a patient comprising: (a) determining if said patient is chemoresistant or develops chemoresistance to one or more chemotherapeutic agents, wherein said one or more chemotherapeutic agents is not gossypol; and (b) co-administering gossypol to said patient if said patient is chemoresistant or develops chemoresistance to treatment with said one or more chemotherapeutic agents. Development of chemoresistance occurs when the patient initially responds to chemotherapy (e.g., tumor shrinkage occurs), but repeated administration of the chemotherapeutic agent(s), diminishes the effectiveness of said chemotherapy such that said chemotherapy is no longer effective at treating the cancer.

In additional embodiments, gossypol is administered to the patient more than once, e.g., two, three, four, five, six, seven, eight, nine, ten, fifteen, twenty, or thirty times, or more.

In additional embodiments, one or more of the chemotherapeutic agents is a Bcl family protein inhibitor. In one such embodiment, the Bcl family protein is selected from the group consisting of Bcl-2, Bcl-xL, and Mcl-1. In another such embodiment, the Bcl family inhibitor is selected from the group consisting of ABT-263 and ABT-737.

In another aspect, the present invention provides a method for determining whether or not Bcl-2 family protein-mediated resistance to certain chemotherapeutic agents in a can be overcome in a patient comprising: (a) obtaining a biological sample from said patient; (b) determining if cancer cells in said biological sample are resistant to apoptosis mediated by one or more chemotherapeutic agents wherein said one or more chemotherapeutic agents do not comprise gossypol; and (c) determining if addition of gossypol decreases said resistance to apoptosis mediated by said one or more chemotherapeutic agents.

In additional embodiments, the method further comprises co-administering gossypol and said one or more additional chemotherapeutic agents to said patient. In additional embodiments, the Bcl-2 family protein is Mcl-1.

In certain embodiments, the methods of the present invention comprising detecting a biomarker in a patient in vivo, e.g., using PET.

In other embodiments, the methods of the present invention comprise detecting a biomarker (a biomarker may comprise one or more biological compounds) in vitro, e.g., from a biological sample taken from a patient. In one embodiment, the biological sample is a tissue sample. Biological samples can be taken from a patient using a variety of conventional techniques that are well within the scope of the ordinary knowledge of a clinical practitioner.

Any tissue sample from a subject may be used. Examples of tissue samples that may be used include, but are not limited to, breast, prostate, ovary, colon, lung, endometrium, stomach, salivary gland or pancreas. The tissue sample can be obtained by a variety of procedures including, but not limited to surgical excision, aspiration, or biopsy. The tissue may be fresh or frozen. In one embodiment, the tissue sample is fixed and embedded in paraffin or the like.

The tissue sample may be fixed (i.e. preserved) by conventional methodology (See e.g., “Manual of Histological Staining Method of the Armed Forces Institute of Pathology,” 3^{rd} edition (1960) Lee G. Luna, H T (ASCP) Editor, The Blakston Division McGraw-Hill Book Company, New York; The Armed Forces Institute of Pathology Advanced Laboratory Methods in Histology and Pathology (1994) Ulreka V. Mikel, Editor, Armed Forces Institute of Pathology, American Registry of Pathology, Washington, D.C.). One of skill in the art will appreciate that the choice of a fixative is determined by the purpose for which the tissue is to be histologically stained or otherwise analyzed. One of skill in the art will also appreciate that the length of fixation depends upon the size of the tissue sample and the fixative used. By way of example, neutral buffered formalin, Bouin's or paraformaldehyde, may be used to fix a tissue sample.

Generally, the tissue sample is first fixed and is then dehydrated through an ascending series of alcohols, infiltrated and embedded with paraffin or other sectioning media so that the tissue sample may be sectioned. Alternatively, one may section the tissue and fix the sections obtained. By way of example, the tissue sample may be embedded and processed in paraffin by conventional methodology (See e.g., “Manual of Histological Staining Method of the Armed Forces Institute of Pathology,” 3^{rd} edition (1960) Lee G. Luna, H T (ASCP) Editor, The Blakston Division McGraw-Hill Book Company, New York). Examples of paraffin that may be used include, but are not limited to, Paraplast, Broloid, and Tissuemay. Once the tissue sample is embedded, the sample may be sectioned by a microtome or the like (See e.g., “Manual of Histological Staining Method of the Armed Forces Institute of Pathology,” 3^{rd} edition (1960) Lee G. Luna, H T (ASCP) Editor, The Blakston Division McGraw-Hill Book Company, New York). By way of example for this procedure, sections may range from about three microns to about five microns in thickness. Once sectioned, the sections may be attached to slides by several standard methods. Examples of slide adhesives include, but are not limited to, silane, gelatin, poly-L-lysine and the like. By way of example, the paraffin embedded sections may be attached to positively charged slides and/or slides coated with poly-L-lysine.

If paraffin has been used as the embedding material, the tissue sections are generally deparaffinized and rehydrated to water. The tissue sections may be deparaffinized by several conventional standard methodologies. For example, xylenes and a gradually descending series of alcohols may be used (See e.g., “Manual of Histological Staining Method of the Armed Forces Institute of Pathology,” 3^{rd} edition (1960) Lee G. Luna, H T (ASCP) Editor, The Blakston Division McGraw-Hill Book Company, New York). Alternatively, commercially available deparaffinizing non-organic agents such as Hemo-De® (CMS, Houston, Tex.) may be used.

### EXAMPLES

### Example 1

### Materials and Methods

Cell Lines and antibodies: 2LMP, MDA-MB-231, MDA-MB-468, MCF-7, PC-3, DU145, 22Rv-1, LNCap, MCF-12F cell lines were purchased from American Type Culture Collection (ATCC, Manassas, Va.) and HMEC from Lonza (Base1, Switzerland). FF fibroblast and MDA-MB-436 cell line were obtained from the University of Michigan, Ann Arbor, Mich. Wild type MEF and Bax/Bak double knock out cell lines which are immortalized by transfection with a plasmid containing SV40 genomic DNA were obtained from the Howard Hughes Medical Institute, Boston, Mass. HMEC and MCF-12F cells were maintained in specific medium recommended by manufacturer or previous study (Hussain-Hakimjee et al., Carcinogenesis 27:551 (2006)). The remaining cell lines were cultured in medium supplemented with 10% FBS and 1% penicillin/streptomycin, at 37° C. in a humidified 5% CO₂ incubator. Antibodies against Noxa, Puma, p53 and c-Myc were purchased from EMD Biosciences (Gibbstown, N.J.), Bax, Mcl-1 from Santa Cruz (Santa Cruz, Calif.), Bak from Upstate (Millipore, Billerica, Mass.), 13-Actin from Sigma (St Louis, Mo.), PARP, Bcl-xL, Bcl-2, Bad, Bid, Bim, Caspase-3 and -7 from cell signaling technology (Danvers, Mass.), Caspase-8 and -9 from Stressgen (Ann Arbor, Mich.). HIF-1α from Affinity Bioreagents (Golden, Colo.), E2F1 from Zymed (San Francisco, Calif.),

Reagents: (−)-Gossypol was dissolved in DMSO at a stock concentration at 20 mM. Control samples, indicated as untreated samples, were incubated only with DMSO.

Cell Growth, Cell Death, and Apoptosis: Cell growth inhibition by (−)-gossypol was determined in a water-soluble tetrazolium (WST)-based assay, as described previously (Shangary et al., Proc. Natl. Acad. Sci. USA 105:3933 (2008)). Cell viability was determined by trypan blue due exclusion as described (Shangary et al., Proc. Natl. Acad. Sci. USA 105:3933 (2008)). Experiments were done in triplicate. To examine apoptosis, cells were stained with Annexin V and PI and analyzed by flow cytometery using an Annexin V-FITC staining kit (Roche Applied Science, Indianapolis, Ind.) according to manufacturer's instructions. Briefly, cells were treated and collected by centrifugation, washed with PBS once and then stained with annexinV-FITC and PI for 15 min at room temperature in the dark. Stained cells were analyzed in a FACS caliber flow cytometer. AnnexinV positive cells are considered as apoptotic cells.

RNA interference: Gene specific double-stranded RNA oligonucleotide targeting Noxa, Puma, c-Myc, HIF1-α, E2F1#2, Bax, Bak, were purchased from Dharmacon RNAi technologies as Sigenome on-targetplus SMARTpool duplex (Lafayette, Colo.). SiRNA of E2F#1 was purchased from Santa Cruz. As a negative control, siRNA against GFP target sequence 5′-aagacccgcgccgaggtgaag-3′ was employed (Qiagen, Valencia, Calif.). Transfections were performed using Lipofectamine RNAiMAX, according to manufacturer's instructions (Invitrogen, Carlsbad, Calif.). (−)-Gossypol was added 24 h after transfection and cells were harvested after 18 h treatment of (−)-gossypol. Three independent knock down experiments were performed for each gene.

Western blot assay: Proteins were harvested in RIPA buffer, then electrophoresed on 4-20% gradient SDS-PAGE gels (Invitrogen, Grand Island, N.Y.) and transferred to PVDF membranes. The membranes were blocked in 5% milk and then incubated with specific primary antibody, followed by secondary antibody. The signals were visualized with the chemiluminescent HRP antibody detection reagent (Deville Scientific, Metuchen, N.J.).

Real-Time PCR: Total RNA was extracted by RNA miniprep kit (Sigma) from cells treated with or without of (−)-gossypol. cDNA was synthesized from 2 μg of RNA using cDNA reverse transcription kits (Applied Biosystems). Next, Taqman gene expression assays were performed using Noxa (Hs00560402_ml), Puma (Hs00248075_ml), Mcl-1 (Hs00172036_ml) gene specific primers/probe sets for real-time PCR in a ABI7900HT PCR machine. GAPDH (Hs99999905_ml) was used for normalization. Relative quantitation of mRNA was calculated by comparative cycle threshold (Ct) method.

Communoprecipitation: The cells were harvested in 1% CHAPs buffer (10 mM HEPES pH7.4, 2.5 mM EDTA, 150 mM NaCl, 1% w/v CHAPs). Mcl-1 was immunoprecipitated by incubation with anti-Mcl-1 antibody overnight at 4° C. on a rotator, followed by co-incubation with protein G plus and A-coupled agarose beads (Santa Cruz) for 1 h at 4° C. Supernatant were harvested and beads were washed three times with CHAPs buffer. Proteins in the immunocomplexes were eluted from the agarose beads by boiling in 2×SDS loading buffer (Novagen, EMD Biosciences) at 95° C. for 5 min and subjected to western blot analysis.

Fluorometric caspase activity assays: Cells were harvested in cell lysis buffer provided by Biovision research products (Mountain View, Calif.). 20 μg of protein was evaluated for caspase activity by incubating with fluorogenic caspase-3 or -9 substrate (EMD Biosciences) in reaction buffer (Biovision). Detection of cleavage of substrate Ac-DEVD-AFC (preferentially cleaved by caspase-3) and Ac-LEHD-AFC (preferentially cleaved by caspase-9) were measured at a λexc=400 nm and λem=505 nm using a Telcan Ultra plate reader (Männedorf, Switzerland). Experiments were performed in triplicate.

Abbreviations used in Figures: cPARP (cleaved PARP), CASP (caspase), cCASP (cleaved caspase).

### Example 2

### (−)-Gossypol Induces Apoptosis in Both Bax/Bak-Dependent and Independent Manners

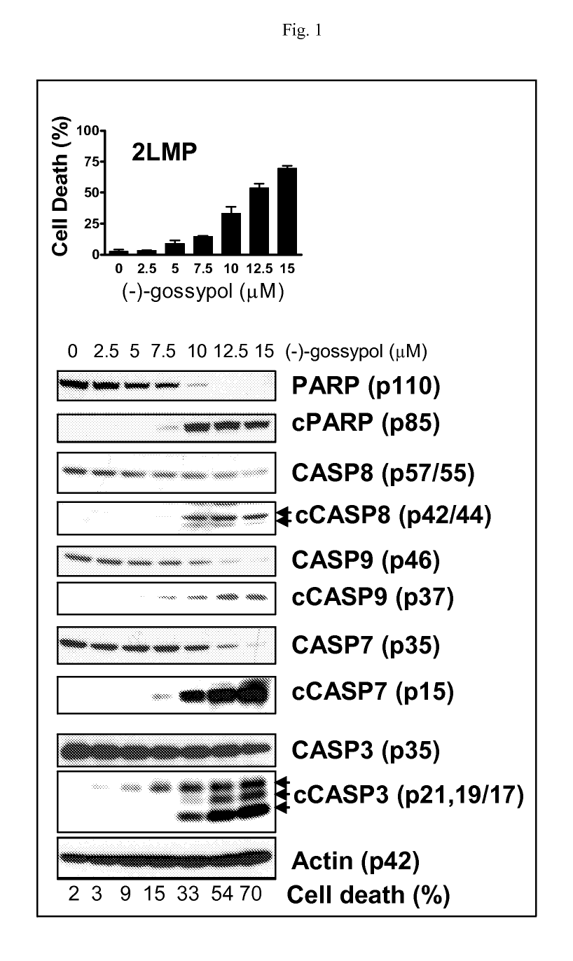

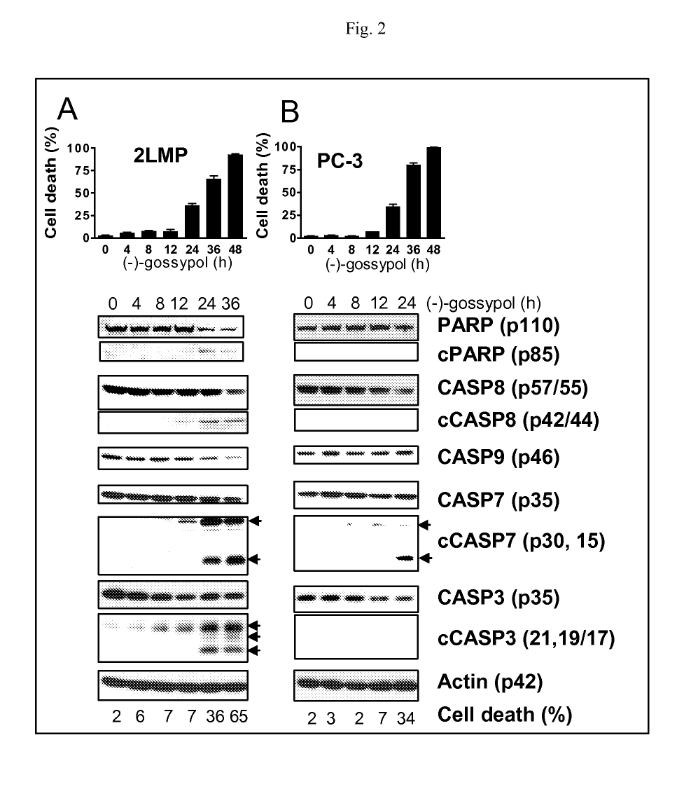

The ability of (−)-gossypol to induce cell death in the MDA-MB-231 (2LMP) human breast cancer and the PC-3 androgen-independent human prostate cancer cell lines was examined along with caspase processing and PARP cleavage. In both cancer cell lines, (−)-gossypol effectively induced cell death in a dose-dependent manner. For example, (−)-gossypol at 10 μM for 24 hour-treatment caused 30-40% of cells to undergo cell death in both 2LMP (FIG. 1) and PC-3. Since (−)-gossypol at 10 μM for 24 h treatment was effective, this dose was selected for investigation of the time-dependence in cell death induction. The induction of cell death in both cancer cell lines is also time-dependent (FIGS. 2A and B). Although no significant cell death was observed at time-points earlier than 12 h, approximately 40%, approximately 70% and nearly 100% of cells lost their viability at 24, 36 and 48 h time-points, respectively, in both 2LMP and PC-3 cell lines.

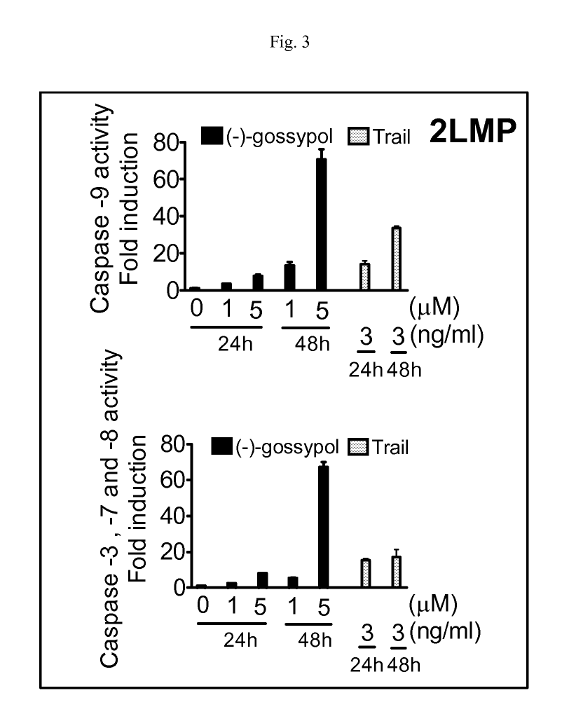

In the 2LMP cell line, cell death induction was associated with a decrease of procaspases, including initiator caspase-9 and -8, and effector caspase-3 and -7 and with a clear increase of cleaved form of caspase-8, -3 and -7. (FIGS. 1 and 2A) Consistent with the western blotting analysis, the activity of caspase-9 and -3/-7 in 2LMP cells was induced by 7 fold after 24 h treatment with 5 μM of (−)-gossypol, and >60 fold after 48 h treatment (FIG. 3). Furthermore, a decrease of Poly (ADP-ribose) polymerase (PARP) and an increase of cleaved PARP was observed at 24 h time point, concurrent with cleavage of caspases and significant cell death induction (FIG. 1 & FIG. 2A).

In the PC-3 cell line, there were minimal or undetectable levels of cleaved caspases and PARP, although there was some decrease in the procaspase-8 and -3 (FIG. 2B). Thus, while (−)-gossypol was equally effective in induction of cell death in the 2LMP and PC-3 cell lines, the levels of cleaved caspases and PARP were different.

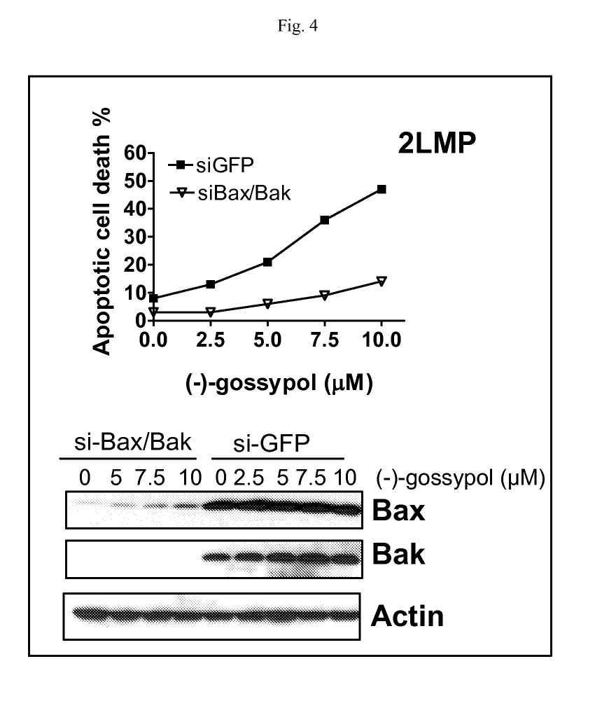

(−)-Gossypol has been shown to induce mitochondrial damage (Benz et al., Mol Pharmacol 37:840 (1990)). Thus, the involvement of mitochondria in apoptosis induction by (−)-gossypol was investigated. Since Bax and Bak are key regulators in mitochondria-mediated apoptosis, the role of Bax and Bak was determined using small interfering RNA (siRNA). Double knockdown of Bax and Bak effectively attenuated gossypol-induced apoptosis in 2LMP cells at all the concentrations tested (FIG. 4). For example, approximately 70% of apoptosis induction by 10 μM of (−)-gossypol was blocked by double knockdown of Bax/Bak. Consistent with significantly reduced apoptosis by (−)-gossypol, the double knockdown of Bax/Bak also abolished PARP cleavage in the 2LMP cells.

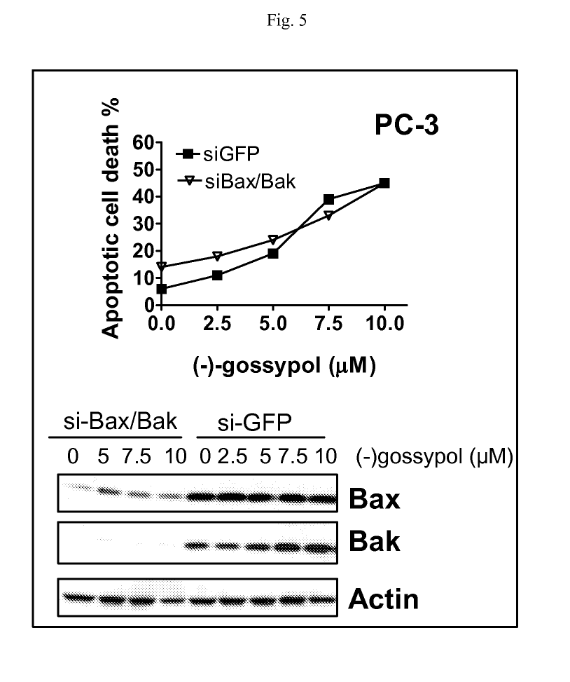

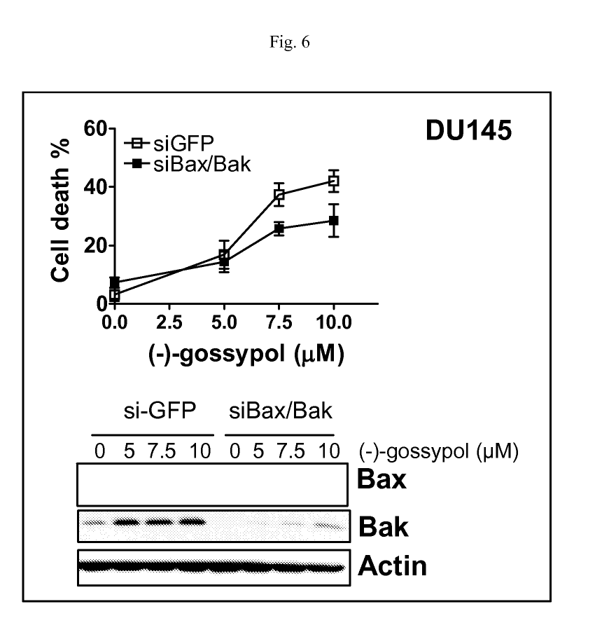

In contrast, the double knockdown of Bax/Bak has no effect on apoptosis induction by (−)-gossypol in the PC-3 cell line (FIG. 5). However, the double knockdown of Bax/Bak in DU145, another androgen-independent prostate cancer cell line, modestly reduced apoptosis induction by (−)-gossypol (FIG. 6). Western blotting confirmed that the levels of Bax and Bak proteins were decreased by >80% by their respective siRNA in all three cell lines (FIGS. 4, 5 and 6) except the endogenous level of Bax is undectable in DU145 cells.

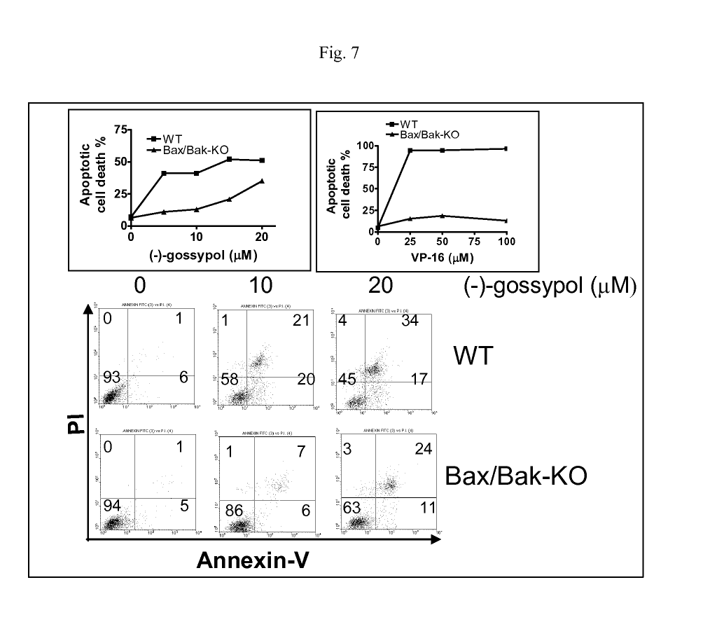

To further confirm the role of Bax/Bak in (−)-gossypol-induced cell death, we employed Bax/Bak double knock-out MEF cells (MEF Bax/Bak KO). At the concentrations of 5 and 10 μM, (−)-gossypol induced 29% and 33% of wild-type MEF cells to undergo apoptosis, respectively, compared to 11% and 13% in MEF Bax/Bak KO cells (FIG. 7). At 20 μM of (−)-gossypol, cell death was reduced in the Bax/Bak double knock-out by 25%. In comparison, induction of cell death by VP-16 was blocked in the MEF Bax/Bak double knock-out at all the concentrations tested, consistent with a previous report (Wei et al., Science 292:727 (2001)).

Hence, (−)-gossypol induces apoptosis in both Bax/Bak-dependent and independent manners. In 2LMP cells, induction of apoptosis by (−)-gossypol is largely dependent upon Bax/Bak and is associated with the activation of caspases and PARP cleavage. In PC-3 cells, induction of apoptosis by (−)-gossypol is independent of Bax/Bak, which is consistent with the minimal cleavage of caspases and PARP cleavage. These two different modes of apoptosis co-exist in DU145 cells. In MEF cells, cell death induction by (−)-gossypol at 15 μM or lower appears to be largely dependent upon Bax/Bak, whereas at 20 μM, the Bax/Bak independent cell-death induction also plays a role.

### Example 3

### (−)-Gossypol Up-Regulates Noxa and Puma in Tumor Cells

The levels of a number of Bcl-2 family members and their interactions have been shown to be regulated by (−)-gossypol (see, for example, Xu et al. Molecular Cancer Therapeutics 4:197 (2005); Oliver et al., Clin Cancer Res 10:7757 (2004); Mohammad et al., Mol Cancer Ther 4:13 (2005); Huang et al., Anticancer Res 26:1925 (2006)). However, their role in (−)-gossypol-induced apoptosis has not been elucidated. To determine the key factors and their role in (−)-gossypol-induced apoptosis, the level of Bcl-2 family proteins and other anti-apoptotic factors were investigated in both 2LMP and PC-3 cells.

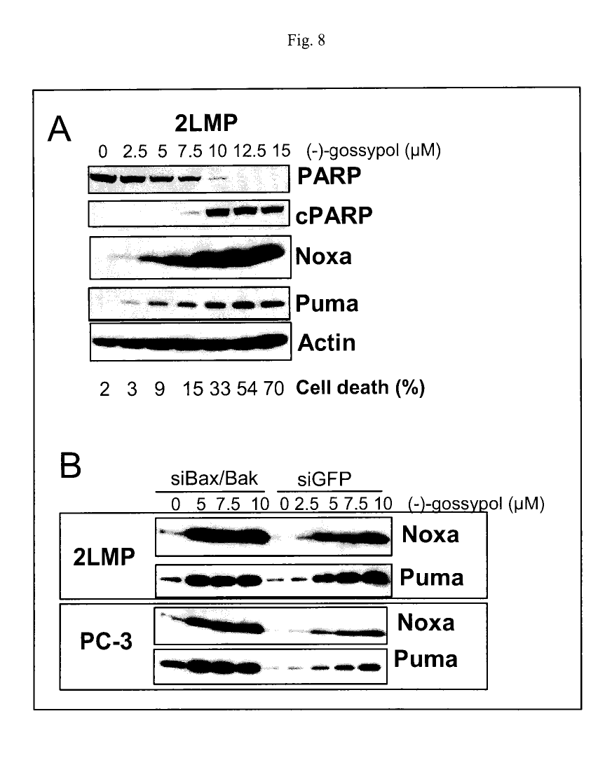

Both Noxa and Puma proteins are significantly upregulated after treatment with 10 μM of (−)-gossypol in both 2LMP and PC-3 cells in a time-dependent manner. Noxa was clearly induced at as early as 4 h and Puma was upregulated at 8 h. The upregulation of Noxa and Puma is much earlier than cell death induction in both cell lines and earlier than the cleavage of caspases and PARP in the 2LMP cells. In addition, induction of Noxa and Puma by (−)-gossypol was dose-dependent and was clear at concentrations as low as 2.5 μM for 24-h treatment (FIG. 8A). Furthermore, double knock-down of Bax/Bak did not affect the induction of Noxa/Puma in both cell lines (FIG. 8B).

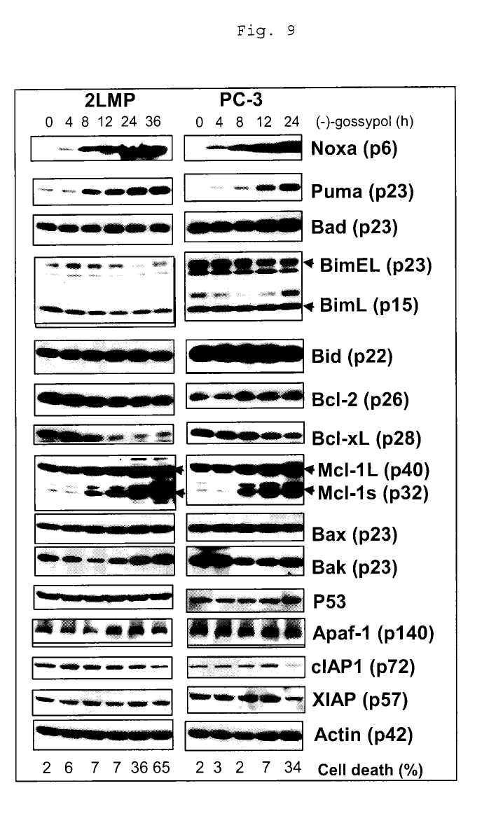

A significant down-regulation of Bcl-xL in the 2LMP cells at 12 h time-point before significant cell death took place at 24 h and some decrease of XIAP and cIAP1 proteins at 24 h or at later time-points (FIG. 9) was also observed. Although there was some increase in the long form of Mcl-1 protein, dramatic induction of the short forms of Mcl-1 was observed in both cell lines as early as 8-h time point. In contrast to the long form of the Mcl-1 protein which functions as anti-apoptotic factor, the short forms functions as pro-apoptotic molecules (Bae et al., J Biol Chem 275:25255 (2000)). No significant change was observed for pro-apoptotic proteins such as Bax, Bak, Bad, Bid, BimL and Apaf-1 in both cell lines, whereas there was some decrease with BimEL (FIG. 9).

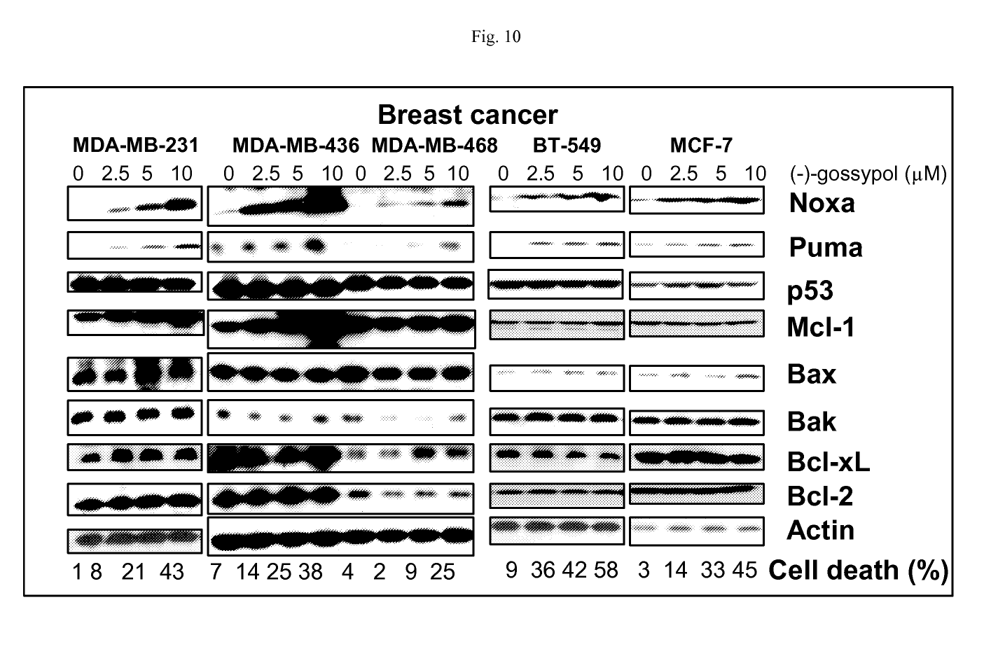

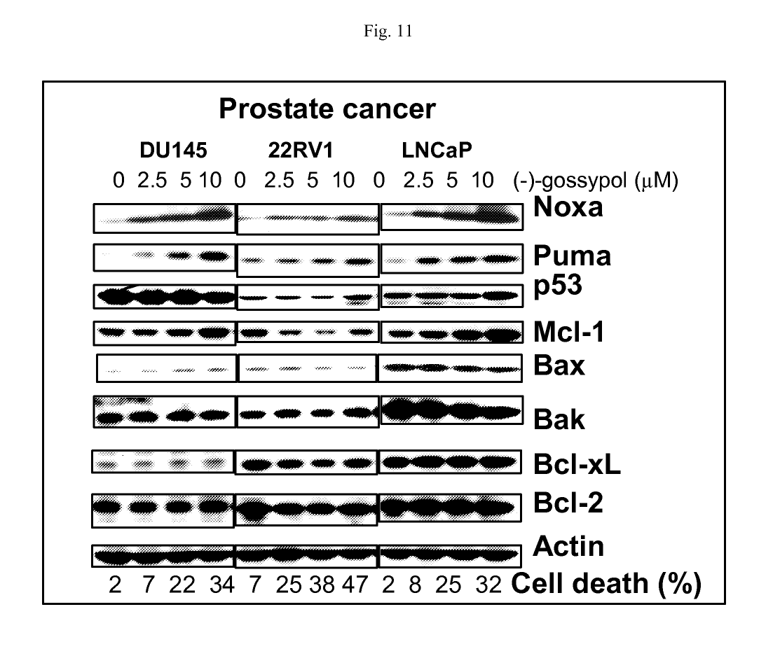

To investigate if the dramatic upregulation of Noxa and Puma by (−)-gossypol is common in tumor cells, their induction in additional 5 breast (MDA-MB-231, MDA-MB-436, MDA-MB-468, BT-549 and MCF-7) and 3 prostate (22Rv1, DU145 and LNCaP) cancer cell lines was examined (FIG. 10 & FIG. 11). Western blotting analysis showed that in each of these 8 additional cancer cell lines, Noxa was universally upregulated in a dose-dependent manner. In fact, in all these 8 cancer cell lines, a significant upregulation of Noxa was observed with as low as 2.5 μM of (−)-gossypol treated for 24 h. Puma was also universally upregulated with 2.5 or 5 μM of (−)-gossypol treatment for 24 h. Although Bcl-xL was decreased by (−)-gossypol in the 2LMP cells, no significant change was found in these 8 cancer cell lines examined, indicating that this decrease is not a common event. Mcl-1 upregulation was found in certain cell lines, including MDA-MB-231, MDA-MB-436, DU145, and LNCaP but not in others, suggesting that the Mcl-1 upregulation is common but not universal. No significant common change was found for other Bcl-2 protein members.

Taken together, these data show that upregulation of Noxa and Puma by (−)-gossypol is universal in all the breast and prostate cancer cell lines examined. Although Noxa and Puma are two known p53-targeted genes, the induction of Noxa and Puma by (−)-gossypol appears to be p53-independent since majority of these cancer cell lines harbor mutated p53 with the exception of MCF-7, LNCaP, and 22Rv1.

### Example 4

### Noxa and Puma Play an Important Role in Apoptosis Induction by (−)-Gossypol in Tumor Cells

Since Noxa and Puma expression is up-regulated by (−)-gossypol in all cell lines examined, their role in apoptosis induction by (−)-gossypol using siRNA strategy was investigated

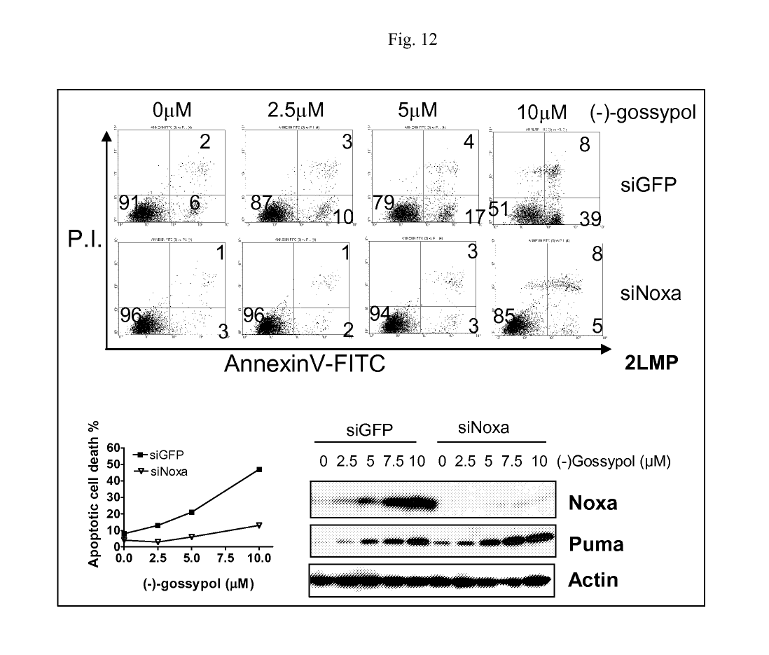

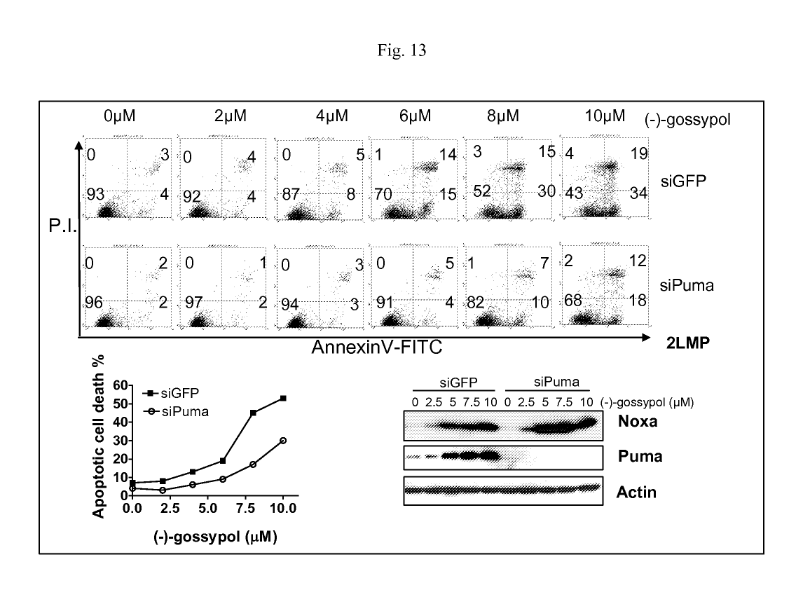

Both basal and (−)-gossypol induced levels of Noxa or Puma were efficiently attenuated by their respective siRNAs in 2LMP and PC-3 cell lines. Of note, the siRNA against Noxa did not negatively affect the basal levels of Puma or vice versa in either cell line, indicating the specificity of these siRNAs (FIGS. 12 and 13).

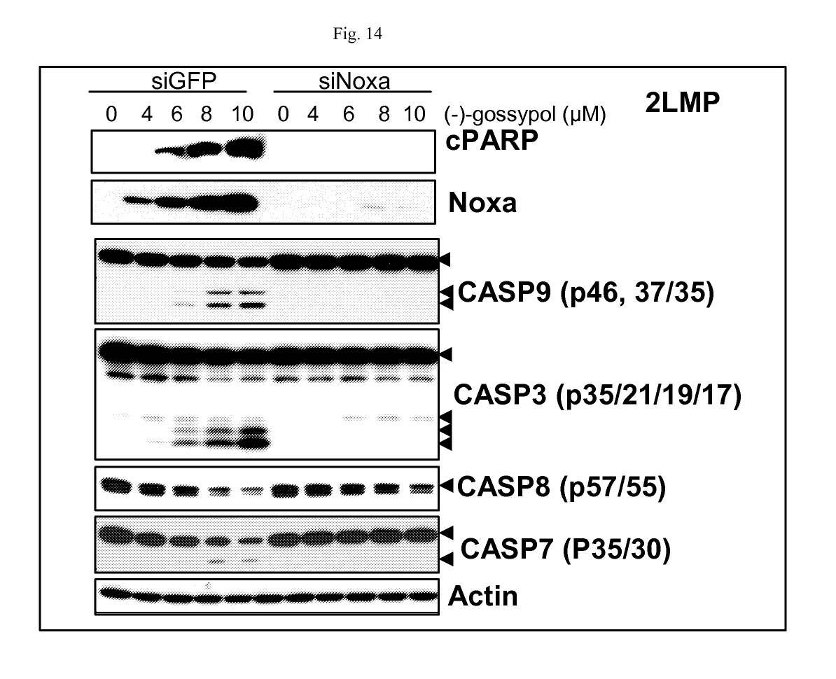

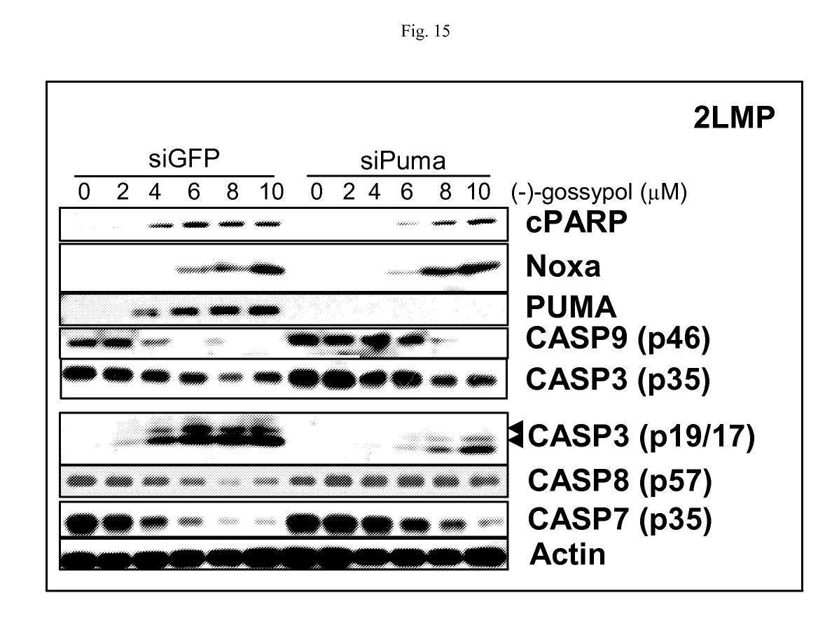

In the 2LMP cell line, knock-down of Noxa effectively reduced >50% of the apoptosis induction by (−)-gossypol in all the concentrations tested in three independent Annexin-V/PI staining experiments (one representative experiment shown in FIG. 12). Similarly, knock-down of Puma also significantly decreased the apoptosis induction by (−)-gossypol by 30-50% (FIG. 13). The attenuation of apoptosis upon knock-down of Noxa or Puma was further confirmed by significant decrease of cleaved PARP and processed caspases (FIGS. 14 and 15).

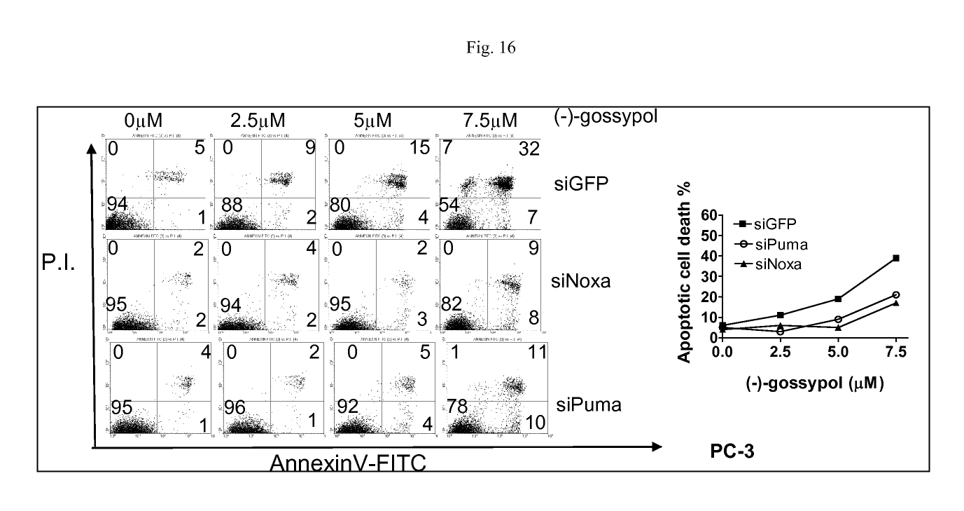

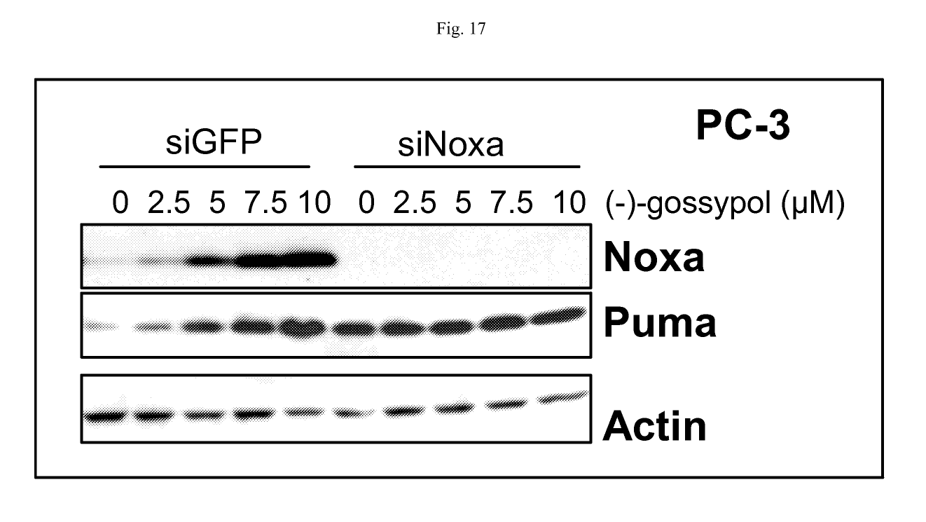

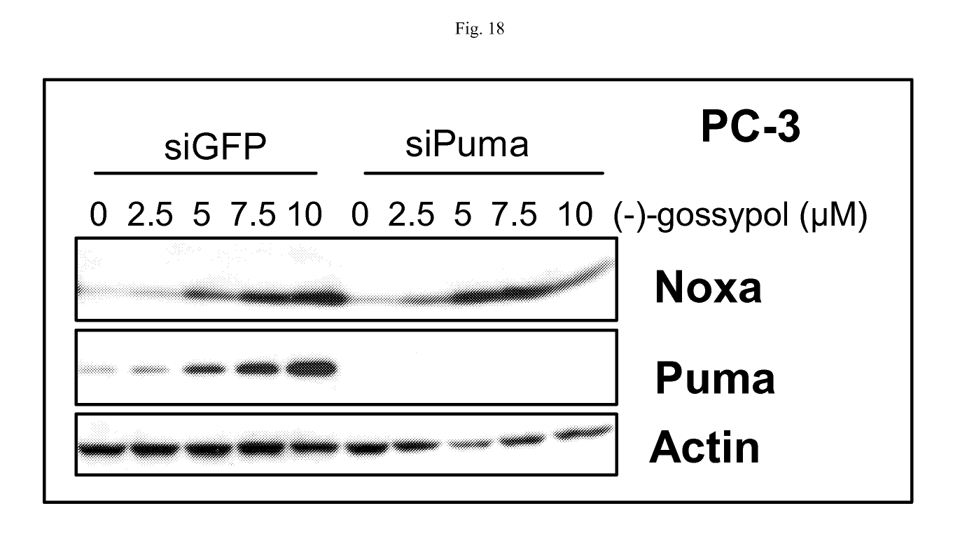

Similar apoptosis reduction upon knock-down of Noxa or Puma was also observed in the PC-3 cell line (FIGS. 16, 17, and 18). Unlike in 2LMP cells, (−)-gossypol induced apoptosis in PC-3 cells in a Bax/Bak-independent manner and accompanied with minimal PARP cleavage and processing of caspases (FIG. 2). Therefore, both Noxa and Puma play a critical role in both Bax/Bak-dependent and -independent apoptosis induction by (−)-gossypol.

### Example 5

### (−)-Gossypol Selectively Upregulates Noxa in Tumor but not in Normal Cells

To investigate if (−)-gossypol selectively induces cell death in tumor cells and if such selectivity can be accounted for by its selective upregulation of Noxa and/or Puma in tumor cells, (−)-gossypol was evaluated in several normal cells or cell lines. These include normal primary breast epithelial cells (HMEC), normal-like human breast epithelial MCF-12F cells, and FF (fibroblast) cells.

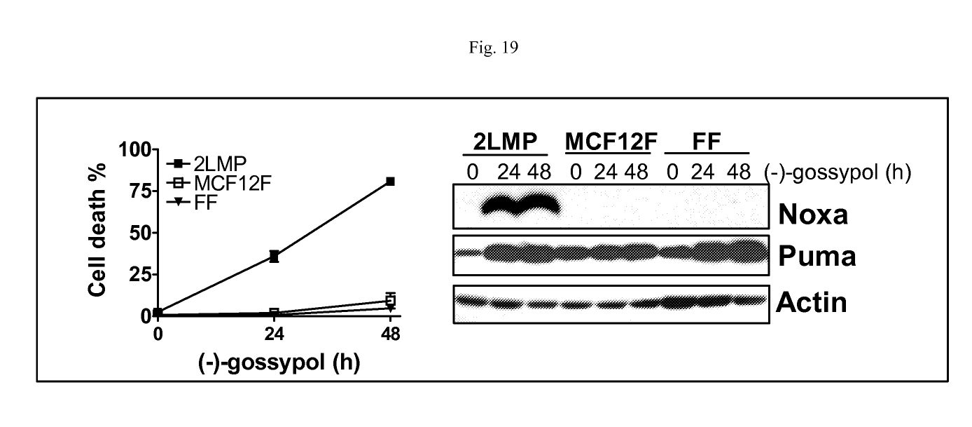

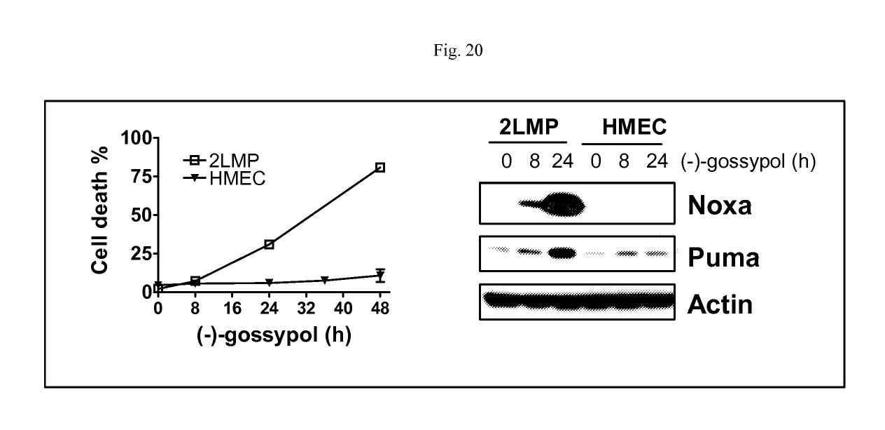

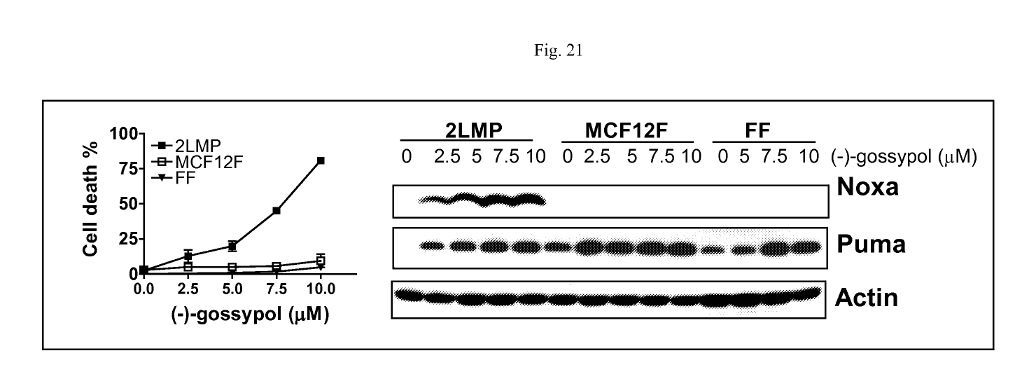

(−)-Gossypol induced minimal cell death in HMEC, MCF-12F and FF cells, as compared to significant cell death induction in 2LMP cells (FIGS. 19, 20 and FIG. 21). Furthermore, Noxa was not induced by (−)-gossypol in these normal cells even with 24-48 h treatment at 10 μM, whereas significant induction of Noxa was observed at 8 h time-point in 2LMP cells (FIG. 20). Interestingly, Puma has high basal levels in MCF-12F and FF cells and can be upregulated by (−)-gossypol in FF and HMEC cells (FIGS. 19, 20 and 21).

These data showed that Noxa is selectively induced in tumor cells by (−)-gossypol. Since Noxa plays a key role in apoptosis induction by (−)-gossypol in tumor cells, the lack of Noxa induction in normal cells could account, at least in part, for its selective toxicity to tumor cells.

### Example 6

### Upregulation of Noxa and Puma by (−)-Gossypol is at the Transcriptional Level

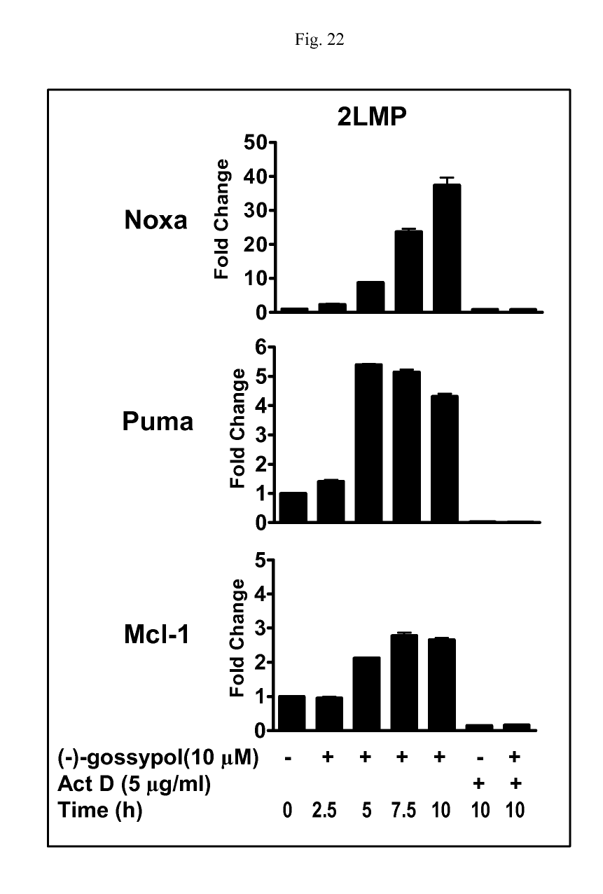

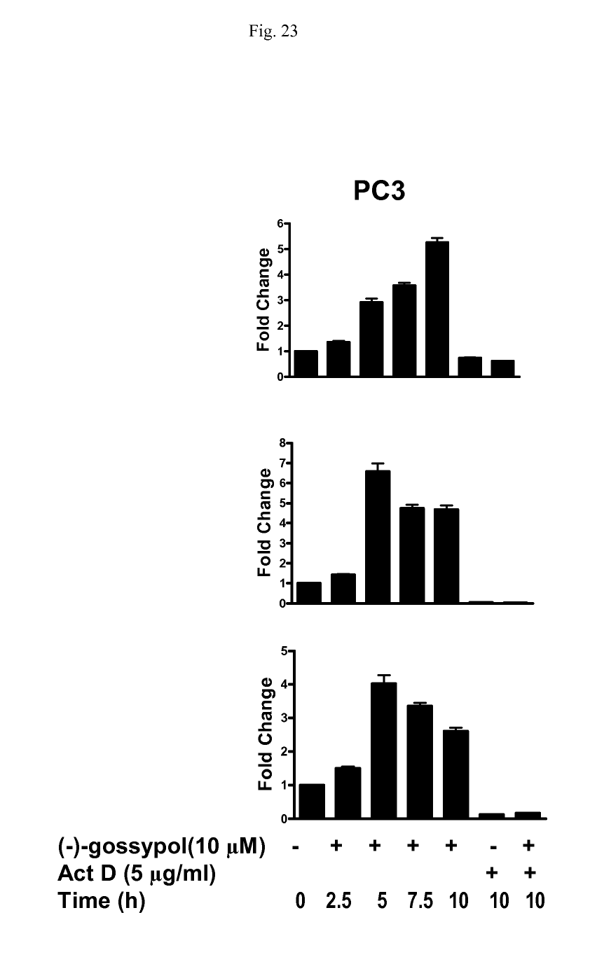

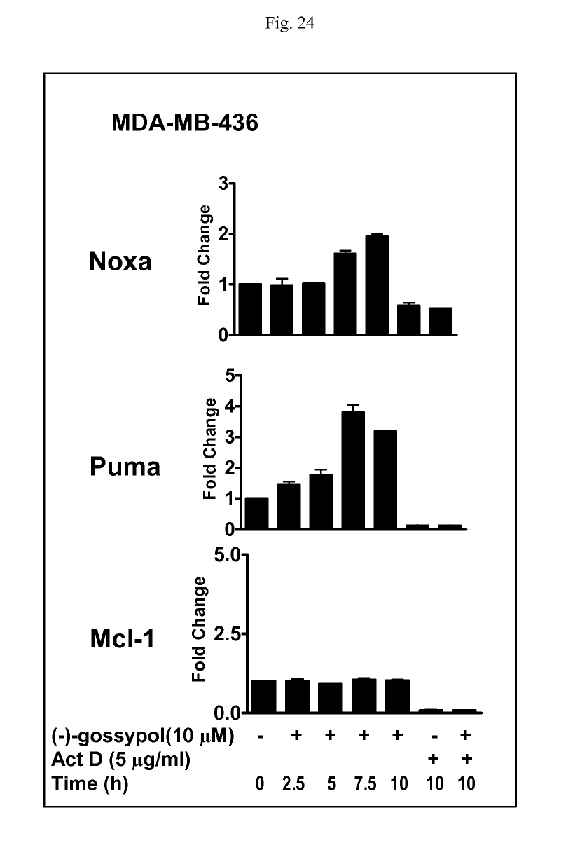

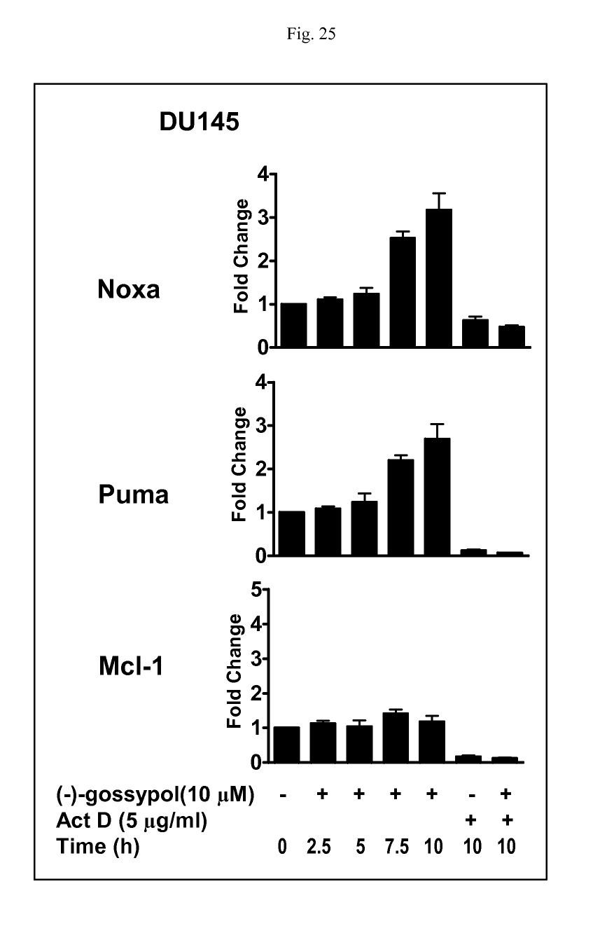

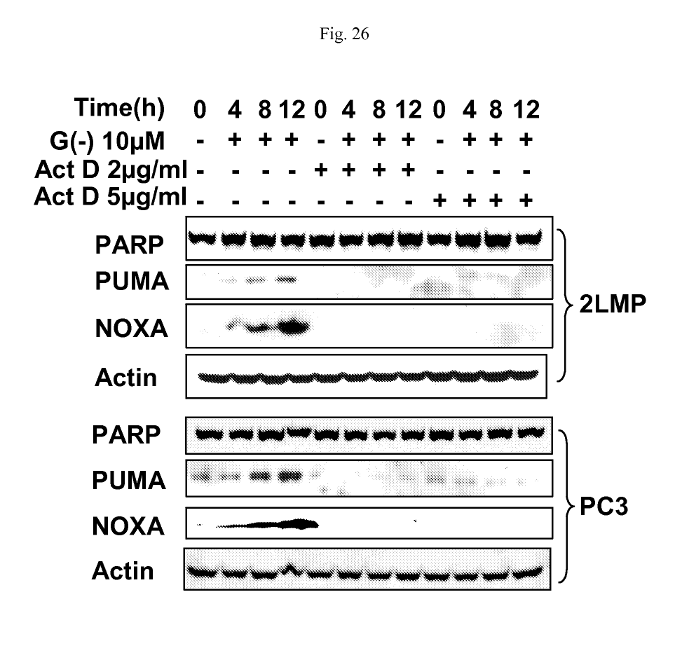

To determine if upregulation of Noxa and Puma by (−)-gossypol is at the transcriptional level, quantitative real-time PCR was performed to determine RNA level of Noxa and Puma after (−)-gossypol treatment in the 2LMP cells. The mRNA levels of Noxa were increased by 2.3-, 9-, 24-, 37-fold at 2.5, 5, 7.5 and 10 h time-points, respectively, and Puma mRNA was increased by 1.4-, 5.4-, 5-, 4.3-fold, respectively (FIG. 22), which was associated with a time-dependent accumulation of Noxa and Puma protein by (−)-gossypol (FIG. 8). Likewise, the mRNA levels of Noxa and Puma were also increased in a time-dependent manner in PC-3, MDA-MB-436 and DU145 cells (FIG. 23 & FIG. 24). There was less than 3-fold increase of the long form of Mcl-1 mRNA at 5-10 h time-points in 2LMP cells, while no significant change was found in MDA-MB-436 and DU145 cells. The cotreatment with actinomycin D, a new RNA synthesis inhibitor, completely blocks the upregulation of these RNAs by (−)-gossypol (FIGS. 22, 23, 24 and 25). Furthermore, actinomycin D significantly blocked (−)-gossypol-upregulated Noxa and Puma proteins in 2LMP and PC-3 cells (FIG. 26), indicating the increase of proteins is mainly due to the increase of their RNA levels. In summary, these data suggested that (−)-gossypol induces upregulation of Noxa, and Puma at the transcriptional level.

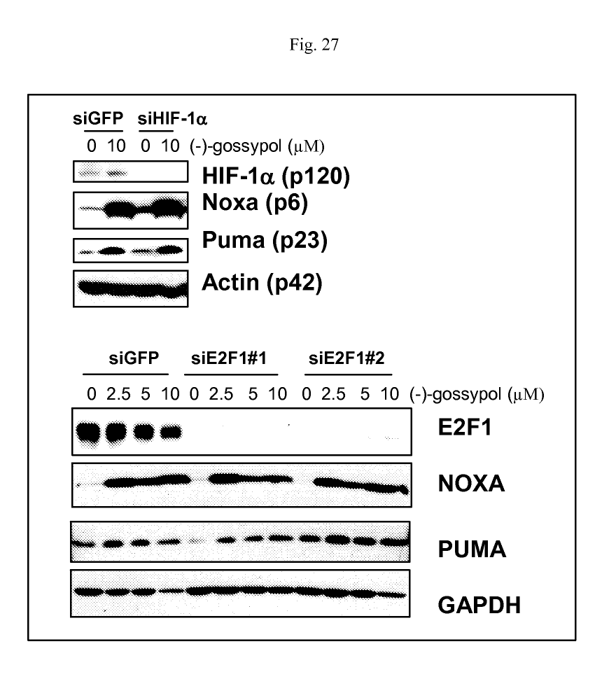

Several transcription factors, which may mediate the upregulation of Noxa and Puma, were also investigated. Since Noxa and Puma are universally upregulated in both p53-mutated and wild-type cancer cells, p53 is unlikely the mediator of Noxa and Puma upregulation. Indeed, knock-down of p53 did not affect the levels of Noxa and Puma, confirming that p53 is not the mediator. Among several candidate transcription factors, HIF-1α was shown to mediate hypoxic cell death by generation of reactive oxygen species (ROS) (Kim et al., J Exp Med 199:113 (2004)) and ROS-dependent mitochondria pathway plays role in gossypol-reduction of tumor growth (Ko et al., Int J Cancer 121:1670 (2007)). In addition, HIF-1α binds to the Noxa promoter region to upregulate Noxa (Kim et al., J Exp Med 199:113 (2004)). Furthermore, E2F1 has important role in regulating apoptosis and upregulation of Noxa and Puma could mediate E2F1-induced apoptosis (Hershko et al., J Biol Chem 279:8627 (2004)). However, downregulation of HIF-1α or E2F1 by siRNA had no effect on the induction of Noxa and Puma by (−)-gossypol, and the subsequent induction of cell death (FIG. 27), indicating that neither HIF-1α nor E2F1 is responsible for the transcriptional upregulation of Noxa and Puma.

### Example 7

### C-Myc Does not Regulate Noxa and Puma but Plays a Critical Role in Gossypol-Induced Apoptosis

A recent study showed that c-Myc mediates Noxa induction by bortezomib, a proteasome inhibitor (Nikiforov et al., PNAS 104:19488 (2007)). Furthermore, c-Myc can also induce the expression of Puma (Jeffers et al., Cancer Cell 4:321 (2003)). Both Noxa and Puma also contain c-Myc-binding sites, making c-Myc a strong candidate transcription factor for the upregulation of Noxa and Puma induced by (−)-gossypol.

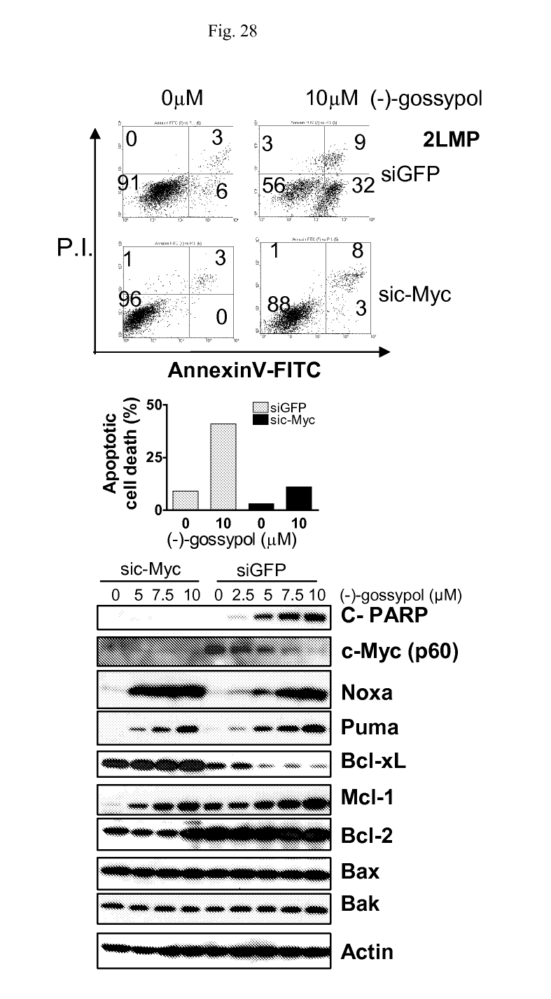

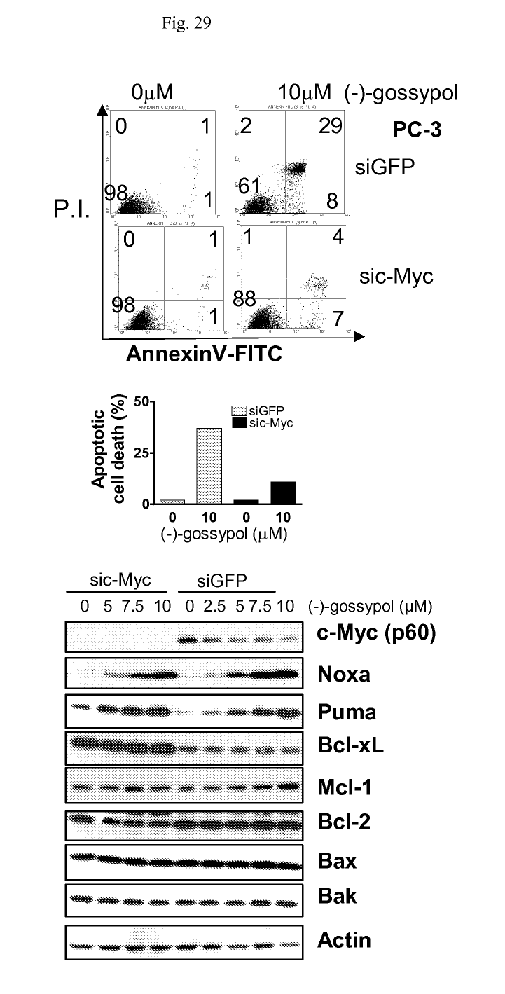

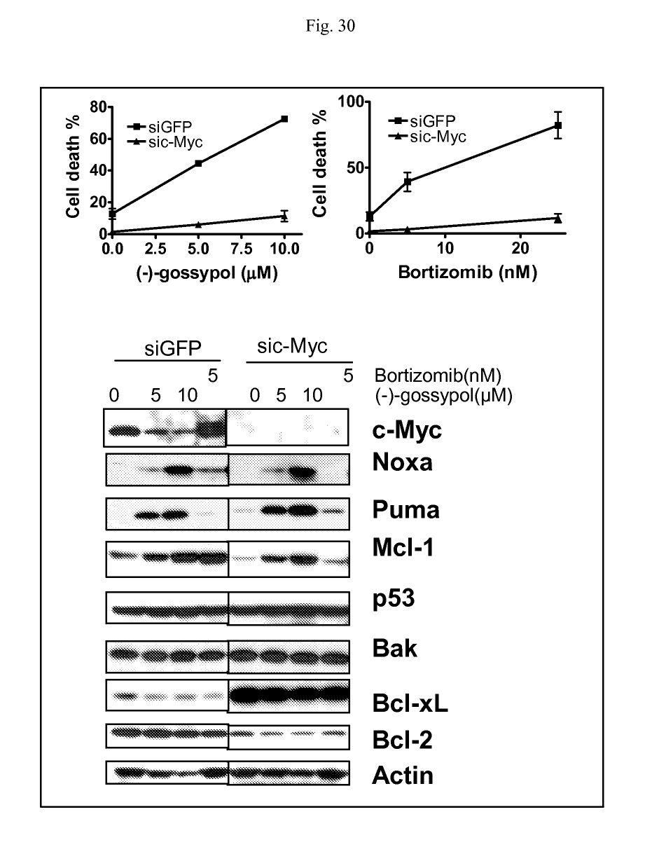

To test this possibility, c-Myc was efficiently knocked down by siRNA in 2LMP and PC-3 cells. While knock-down of c-Myc had no effect on the upregulation of Noxa and Puma by (−)-gossypol, it blocked >70% of cell death induction by 10 μM of (−)-gossypol in both cell lines (FIGS. 28 and 29). The effective blockage of cell death induction by (−)-gossypol upon c-Myc knock-down was also observed in the p53-mutated DU145 cells and p53 wild-type RKO colon cancer cells. Consistent with a previous report in melanoma cell lines (Nikiforov et al., PNAS 104:19488 (2007), knock-down of c-Myc indeed reduced the expression of Noxa induced by bortezomib and blocked cell death in the 2LMP cells (FIG. 30). Therefore, these data showed that although c-Myc is not the transcription factor that mediates Noxa and Puma induction by (−)-gossypol, it plays a central role in apoptosis induction by (−)-gossypol.

To further explore the underlying mechanism of c-Myc in regulation of apoptosis induction by (−)-gossypol, the expression of several other Bcl-2 members which have been identified as c-Myc targeted genes, including Bcl-2, Bcl-xL, Bak and Bax, were examined. There was a decrease of the anti-apoptotic Bcl-2 protein and no significant change on the levels of pro-apoptotic Bak and Bax proteins was observed (FIGS. 28 and 29). However, there was a dramatic increase on the basal level of the anti-apoptotic Bcl-xL protein upon c-Myc knock-down, which was not decreased with the treatment of (−)-gossypol in both 2LMP and PC-3 cells. These data suggested that knock-down of c-Myc protects cancer cells from apoptosis induction by (−)-gossypol at least in part through the dramatic upregulation of the anti-apoptotic Bcl-xL protein.

### Example 8

### (−)-Gossypol Overcomes Resistance to Cell Death Conferred by Mcl-1

Mcl-1 is an antiapoptotic member of the Bcl-2 family proteins that has a short half life of about 30 min to about 3 h and can be rapidly induced by cytokines or growth factors (Chao et al., Mol Cell Biol 18:4883 (1998); Yang-Yen et al., Journal of Biomedical Science 13:201 (2006)). Mcl-1 is overexpressed in many types of human cancer and plays a critical role in protecting cancer cells from induction of apoptosis by a variety of anticancer stimuli (Yang-Yen et al., Journal of Biomedical Science 13:201 (2006); Nijhawan et al., Genes Dev 17:1475 (2003). Furthermore, Mcl-1 confers the resistance of cancer cells to ABT-737, a potent and specific small-molecule inhibitor of Bcl-2 and Bcl-xL but not of Mcl-1 (Letai, Trends Mol Med 11:442 (2005); van Delft et al., Cancer Cell 10:389 (2006); Lin et al., Oncogene 26:3972 (2007); Konopleva et al., Cancer Cell 10:375 (2006)). Since (−)-gossypol strongly upregulates Noxa and Puma, both of which can bind to Mcl-1 and neutralize its anti-apototic function (Chen et al., Mol Cell 17:393 (2005); Certo et al., Cancer Cell 9:351 (2006), (−)-gossypol was evaluated to see if it could effectively overcome the Mcl-1-mediated resistance of cancer cells to ABT-737.

ABT-737 has weak activity in both the 2LMP and PC-3 cell lines with IC₅₀ values of >10 μM in WST cell growth assays, consistent with the high levels of Mcl-1 in these cancer cell lines. Knock-down of Mcl-1 sensitizes both 2LMP and PC-3 cells to cell death induction by ABT-737, confirming the role of Mcl-1 in mediating the resistance of cancer cells to ABT-737 and in agreement with previous studies in other tumor cell lines.

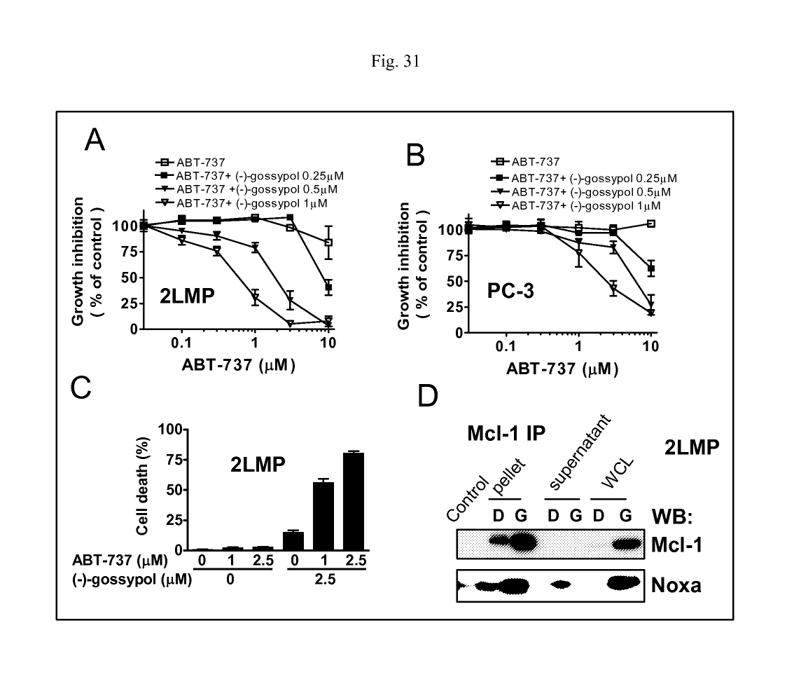

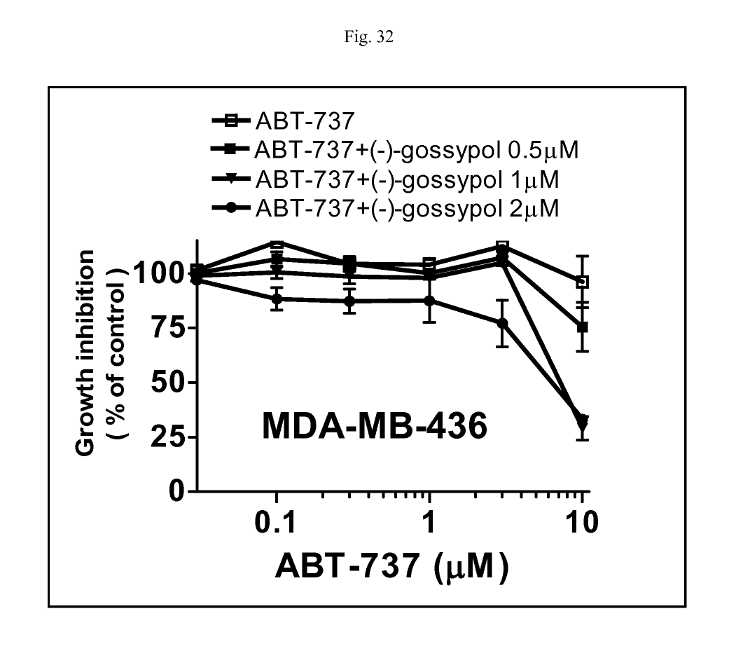

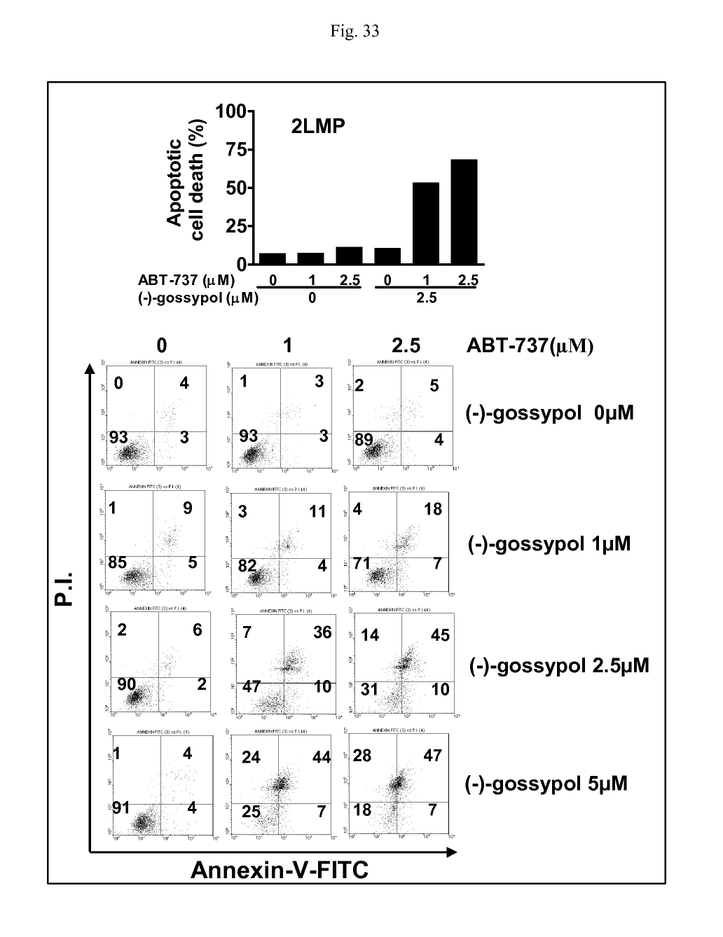

(−)-Gossypol at concentrations as low as 0.5 and 1 μM can effectively sensitize 2LMP, PC-3 and MDA-MB-436 cancer cells to ABT-737 as shown by WST cell growth assay (FIGS. 31A, 31B and 32). Furthermore, while (−)-gossypol at 2.5 μM for 48 h treatment killed only 14% of 2LMP cells and ABT-737 at 1 and 2.5 μM had no effect, the combination induced 60% and 80% of cells to lose their viability, respectively (FIG. 31C). This dramatic synergistic effect was further confirmed in apoptosis analysis using Annexin-V/PI staining by flow cytometry (FIG. 33).

Whether or not the ability of (−)-gossypol to sensitize cancer cells to ABT-737 was due to specific targeting of Mcl-1 through upregulation of Noxa and Puma was also investigated. To this end, coimmunoprecipitation experiments were performed in the 2LMP cell line to examine the complex formation of Noxa and Puma with Mcl-1 upon (−)-gossypol treatment. Mcl-1 was efficiently pulled down and Noxa was found to be in the immunocomplex (FIG. 31D). Furthermore, Noxa but not Mcl-1 was also detected in the supernatant, suggesting that there was excess amount of Noxa to saturate Mcl-1 protein. Interestingly, although Puma was also upregulated by (−)-gossypol, there was no detectable level of Puma in the complex.

Taken together, these data indicate that upregulation of Noxa and Puma by (−)-gossypol can overcome Mcl-1 mediated apoptosis resistance of cancer cells to ABT-737, and Noxa plays a dominant role to bind to and neutralize Mcl-1 protein.

It will be readily apparent to one of ordinary skill in the relevant arts that other suitable modifications and adaptations to the methods and applications described herein may be made without departing from the scope of the invention or any embodiment thereof. It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the claims. Having now described the present invention in detail, the same will be more clearly understood by reference to the following examples, which are included herewith for purposes of illustration only and are not intended to be limiting of the invention.

1. A method of selecting a patient with a disease, condition, or disorder for treatment with gossypol, the method comprising:
(a) determining the expression level of a biomarker selected from the group consisting of c-Myc, Mcl-1, and combination of c-Myc and Mcl-1 in said patient; and
(b) administering gossypol to said patient if said patient has an elevated expression level of said biomarker relative to the normal expression level of said biomarker.

2. A method for treating or ameliorating a disease, condition, or disorder in a patient, the method comprising:
(a) determining the expression level of a biomarker selected from the group consisting of c-Myc, Mcl-1, and combination of c-Myc and Mcl-1 in said patient; and
(b) administering gossypol to said patient if said patient has an elevated expression level of said biomarker relative to the normal expression level of said biomarker.

3. The method of claim 2, wherein said biomarker is c-Myc.

4. The method of claim 2, wherein said biomarker is Mcl-1.

5. The method of claim 2, wherein said disease, condition, or disorder is selected from the group consisting of hyperproliferative disease, autoimmune disease, inflammatory disease, infectious disease, degenerative disease, and vascular disease.

6. The method of claim 5, wherein said disease, condition, or disorder is a hyperproliferative disease.

7. The method of claim 6, wherein said hyperproliferative disease is cancer.

8. The method of claim 7, wherein said cancer is selected from the group consisting of breast cancer, prostate cancer, lymphoma, skin cancer, pancreatic cancer, colon cancer, malignant melanoma, ovarian cancer, brain cancer, primary brain carcinoma, head-neck cancer, glioma, glioblastoma, liver cancer, bladder cancer, non-small cell lung cancer, head carcinoma, neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, retinoblastoma, and a combination of two or more of said cancers.

9. The method of claim 8, wherein said cancer is prostate cancer.

10. The method of claim 2, wherein said gossypol is (−)-gossypol.

11. The method of claim 2 further comprising co-administering one or more additional therapeutic agents.

12. The method of claim 11, wherein said one or more additional therapeutic agents comprise one or more additional anticancer agents.

13. The method of claim 12, wherein said one or more additional anticancer agents comprise at least one chemotherapeutic agent.

14. The method of claim 13, wherein at least one of said one or more chemotherapeutic agents are selected from the group consisting of abraxane, actinomycin D, aldesleukin, alemtuzumab, alitretinoin, allopurinol, altretamine, amifostine, aminoglutethamide, anastrozole, arsenic trioxide, asparaginase, azacitidine, azathioprine, BCG live, bevacizumab, bexarotene, bicalutamide, bleomycin, bortezomib, busulfan, butazolidin, capecitabine, carboplatin, carmustine, celecoxib, chlorambucil, cisplatin, cladribine, clofarabine, cyclophosphamide, cytarabine, dacarbazine, dactinomycin, darbepoetin alfa, daunomycin, daunorubicin, denileukin diftitox, dexamethasone, dexrazoxane, diethylstilbestrol, docetaxel, doxorubicin, dromostanolone propionate, epirubicin, epoetin alfa, estramustine, ethinyl estradiol, etoposide, exemestane, filgrastim, finasteride, floxuridine, fludarabine, fluorouracil, fluoxymesterone, flutamide, fulvestrant, gefitinib, gemcitabine, gemtuzumab, goserelin acetate, hexamethylmelamine, hydroxychloroquine, hydroxyprogesterone caproate, hydroxyurea, ibritumomab, idarubicin, ifosfamide, imatinib, interferon alfa-2a, interferon alfa-2b, interleukin-2, irinotecan, ketoconazole, letrozole, leucovorin, leuprolide, levamisole HCl, lomustine, mechlorethamine, medroxyprogesterone acetate, megestrol acetate, meloxicam, melphalan, mercaptopurine, mesna, methotrexate, methoxsalen, methylprednisolone, metronidazole, misonidazole, mithramycin, mitomycin, mitotane, mitoxantrone, nandrolone phenpropionate, nitrogen mustard, nitroimidazole, nitrosourea, nofetumomab, oblimersen sodium, oprelvekin, oxaliplatin, oxaliplatin, oxyphenbutazone, paclitaxel, pamidronate, pegademase, pegaspargase, pegfilgrastim, pentostatin, phenylbutazone, picoplatin, pipobroman, plicamycin, plicamycin, porfimer sodium, prednisolone, prednisone, procarbazine, procarbazine, quinacrine, raloxifene, rasburicase, rituximab, romidepsin, sargramostim, semustine, streptozocin, talc, tamoxifen, temozolomide, teniposide, testolactone, testosterone propionate, thalidomide, thioguanine, thiotepa, tiripazamine, topotecan HCl, toremifene, tositumomab, trastuzumab, tretinoin, trimethoprim/sulfamethoxazole, uracil mustard, valrubicin, vinblastine, vincristine, vindesine, vinorelbine, zoledronic acid, and a combination of two or more of said chemotherapeutic agents.

15. The method of claim 2, wherein said disease, condition, or disorder is prostate cancer, said biomarker is c-Myc, and said gossypol is (−)-gossypol.

16. The method of claim 15, further comprising co-administering one or more additional chemotherapeutic agents.

17. The method of claim 15, wherein said (−)-gossypol comprises (−)-gossypol acetic acid co-crystals.

18. The method of claim 16, wherein at least one of said one or more chemotherapeutic agents is selected from the group consisting of docetaxel and paclitaxel.

19. A method for overcoming Mcl-1-mediated chemoresistance to chemotherapy in a patient, the method comprising:
(a) determining if said patient is chemoresistant to one or more chemotherapeutic agents, wherein said chemotherapeutic agent is not gossypol; and
(b) co-administering gossypol to said patient if said patient is chemoresistant to treatment with said one or more chemotherapeutic agents.

20. (canceled)

21. The method of claim 19, wherein at least one of said one or more chemotherapeutic agents is a Bcl family protein inhibitor.

22. (canceled)

23. (canceled)
